SEQUENCE LISTING (2 sequences)

SEQ ID NO: 1 - protein, 609 aa, Homo sapiens
  source 1..609: note = Alpha-fetoprotein (AFP) UniProt accession number P02771
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 116 aa, Homo sapiens
  source 1..116: note = hCG UniProt accession number P01215
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

(19) United States
(12) Patent Application Publication
(10) Pub. No.: US 2026/0338730 A1
(43) Pub. Date: Oct. 29, 2026

(54) SPECIFIC AND LABEL-FREE SENSING OF SMALL MOLECULE TARGETS USING BIOLOGICAL TRANSISTORS

(71) Applicants: B.G. NEGEV TECHNOLOGIES AND APPLICATIONS LTD., AT BEN-GURION UNIVERSITY, Beer Sheva (IL); MOR RESEARCH APPLICATIONS LTD., Ramat Gan (IL)

(72) Inventors: Gil SHALEV, Beer Sheva (IL); Rakefet SAMUELI, Beer Sheva (IL); Shubham BABBAR, Beer Sheva (IL); Offer EREZ, Kibbutz Shoval (IL)

(73) Assignees: B.G. NEGEV TECHNOLOGIES AND APPLICATIONS LTD., AT BEN-GURION UNIVERSITY, Beer Sheva (IL); MOR RESEARCH APPLICATIONS LTD., Ramat Gan (IL)

(21) Appl. No.: 19/504,669

(22) PCT Filed: Jul. 23, 2024
(86) PCT No.: PCT/IL2024/050724

§ 371 Date: Jan. 22, 2026

Related U.S. Application Data
(60) Provisional application No. 63/528,392, filed on Jul. 23, 2023.
(60) Provisional application No. 63/537,564, filed on Sep. 11, 2023.
(60) Provisional application No. 63/625,672, filed on Jan. 26, 2024.

(51) Int. Cl. G01N 27/414

(52) U.S. Cl. CPC G01N 27/4145; G01N 27/4148; G01N 2333/575

(57) ABSTRACT

The present disclosure provides bio-transistors for sensing target molecules. More specifically, the present disclosure provides systems and methods for determining the presence and/or quantity of target molecules, specifically, small molecule targets, such as organophosphates or alternatively, naturally produces molecules such as estriol, in a biological sample, thereby providing diagnosis and monitoring of conditions associated with the target molecules.

### TECHNOLOGICAL FIELD

The present disclosure relates to bio-transistors for sensing target molecules. More specifically, the present disclosure provides systems and methods for determining the presence and/or quantity of molecules, specifically of small molecules, particularly, chemical warfare agents or steroid hormones.

### BACKGROUND ART

References considered to be relevant as background to the presently disclosed subject matter are listed below:
1. Samuel, V. R. & Rao, K. J. A review on label free biosensors. Biosens Bioelectron X 11, (2022).
2. US20230022648.
3. Tan, F., Saucedo, N. M., Ramnani, P. & Mulchandani, A. Label-Free Electrical Immunosensor for Highly Sensitive and Specific Detection of Microcystin-LR in Water Samples. Environ Sci Technol 49, 9256-9263 (2015).
4. McAlpine, M. C. et al. Peptide-Nanowire Hybrid Materials for Selective Sensing of Small Molecules. J Am Chem Soc 130, 9583-9589 (2008).
5. Kong, L. et al. Novel pyrenehexafluoroisopropanol derivative-decorated single-walled carbon nanotubes for detection of nerve agents by strong hydrogen-bonding interaction. Analyst 135, (2010).
6. Kong, L. et al. p-Hexafluoroisopropanol phenyl covalently functionalized single-walled carbon nanotubes for detection of nerve agents. Carbon N Y 48, 1262-1270 (2009).
7. Bhattacharyya, I. M. et al. A new approach towards the Debye length challenge for specific and label-free biological sensing based on field-effect transistors. Nanoscale 14, 2837-2847 (2022).
8. Bhattacharyya, I. M. et al. A New Approach toward the Realization of Specific and Label-Free Biological Sensing Based on Field-Effect Devices. Adv Electron Mater 8, (2022).

Acknowledgement of the above references herein is not to be inferred as meaning that these are in any way relevant to the patentability of the presently disclosed subject matter.

### BACKGROUND

Transistor-based sensing has been suggested for specific, label-free, real-time, low cost and quantitative biological and chemical sensing from ultra small samples with high selectivity and sensitivity [1].

Label-free and specific Field-effect transistors (FET)-based biological sensing is based on surface-bound recognition layer composed of receptor molecules with high affinity, selectivity and specificity toward the biomolecular target. On the other hand, FET-based chemical sensing is mostly realized with a surface-bound semipermeable membrane to provide the specificity toward ions other than H⁺. FET-based sensing has important implications and application in various fields ranging from medical diagnostics, homeland security, food industry, agriculture, and others.

The inventors previously developed a biosensor to shorten and optimize the Debye length at an interface between the sensing region and the fluid [2]. This biosensor includes a semiconductor active region; a sensing region configured to contact a fluid; and multiple electrodes that comprise decoupling electrodes and additional electrodes. The decoupling electrodes may be configured, wherein operating in a first mode, to prevent a formation of a top conductive channel within the semiconductor active region; and the additional electrodes are configured, wherein operating in the first mode, to independently control (i) one or more properties of one or more other conductive channels formed within the semiconductor active region, and (ii) a Debye length at an interface between the sensing region and the fluid [2].

In the domains of analytical chemistry and biomedical research, the precise and sensitive detection of small molecules has captured considerable interest owing to its pivotal significance across a spectrum of domains including pharmaceuticals environmental surveillance [Sanford, A et al. Chem Sci 13, 7670-7684 (2022)], medical diagnostics, and the identification of potential biological threats [Thorpe et al., Science (1979) 311, 1595-1599 (2006)]. Small molecules, defined as low-weight organic compounds, encompass metabolites, drugs, toxins, hormones, and signaling molecules [Li, and Kang, Int J Mol Sci 21, 5262 (2020)]. Despite their size, they wield substantial influence over biological processes, necessitating precise detection to unravel intricate molecular mechanisms. Surface plasmon resonance (SPR) [Wang et al., Talanta 198, 350-357 (2019)], and enzyme-linked immunosorbent assay (ELISA) are frequently employed methods for small-molecule detection [Watanabe et al., J Agric Food Chem 61, 12459-12472 (2013)]. However, although SPR is a label-free sensing method, unlike the inherent labeling utilized in ELISA, employing SPR for small molecule immunosensing presents challenges as it mandates high-mass labels, or noble metals to yield signals in sensitive assays. Such labelling techniques have inherent deficiencies, including the potential alteration of ligand structure, which can affect the binding mechanisms and compromise assay accuracy. Additionally, quantifying secondary interactions instead of direct ones could introduce inherent deviations, diminishing assay specificity. Moreover, these techniques often lack the required sensitivity for crucial measurements. Finally, steric hindrance between small molecules and antibodies could arise in the presence of labels. Consequently, there is a growing need for miniaturized, low-cost, real-time, high-end sensing in ultra-small samples to allow the realization of applications ranging from point-of-care and home-care diagnostic to homeland security.

Chemiresistors and Field-effect biosensors (bioFETs), of various novel nanostructures and materials, have been suggested for specific and label-free sensing of small molecules [Nakatsuka et al. Science (1979) 362, 319-324 (2018)]. For small molecule detection, the competitive format (‘displacement mode’) is applied in many cases to overcome the challenge of detecting the changes induced by binding small, sometimes neutral, molecules to the binding sited on the antibody surface [3].

Field-effect biosensors (bioFETs) are promising candidates for specific, label-free, and real-time sensing, and specifically for POC and home-care medical diagnostics. The underlying bioFET sensing mechanism is charge introduction and/or charge redistribution, at the sensing area due to the capture of target molecules by the surface-bound receptor molecules. Recently, bioFETs based on silicon nanowires were reported for label-free sensing of small molecules. McAlpine et al reported sensing of small molecules via the conformational modification and charge redistribution of the receptor molecule [4] introduced by the small molecule. Different receptor molecules have been employed in these works ranging from chemical receptors, peptides/large proteins, and DNA strands.

Organophosphate (OP) sensing stems from the pressing need to address environmental, health, and security challenges. OPs are extensively used in agriculture to control pests and enhance crop yields. The annual use of organophosphorus pesticides, reaching several hundred thousand tons, and their low decomposition rates lead to accumulation in the environment, rivers, groundwater, and food sources, posing risks to human and animal health [Dhamu, et. al. ECS Sensors Plus 2, (2023)]. Moreover, hazardous phased-out and prohibited organophosphorus pesticides can enter the environment through damaged packaging during prolonged storage [Andrianova et al. Electroanalysis 28, (2016)]. Furthermore, OPs are employed in chemical warfare agents, posing significant threats to national security and public safety [Kumar, D. et al. Mater Chem Phys 181, (2016)]. The nerve agent Sarin exemplifies the lethal nature of organophosphorus compounds, and has been employed by terrorists, underscoring the urgent need for inexpensive portable sensors capable of real-time detection and quantification of OP residues with high sensitivity and specificity.

Conventionally, OPs are measured using various analytical techniques, including chromatography, spectroscopy, and immunoassays. Chromatographic methods, such as gas chromatography (GC) and high-performance liquid chromatography (HPLC), offer high sensitivity and selectivity but often require extensive sample preparation and specialized equipment [Kumaran, A. et al. Microchemical Journal 178, 107420 (2022)]. Analytical techniques, like mass spectrometry (MS) and nuclear magnetic resonance (NMR) spectroscopy, provide detailed structural information but are less suitable for rapid on-site analysis [Kumar, P., et al. Biosens Bioelectron 70, (2015)]. Immunoassays, such as enzyme-linked immunosorbent assays (ELISA) and lateral flow assays (LFAs) offer rapid and cost-effective screening but may lack specificity and sensitivity. Additionally, fluorescent-based detection methods have emerged as promising alternatives for OP sensing.

The integration of nanomaterials, such as graphene and carbon nanotubes (CNT), improves sensor performance by increasing surface area and facilitating charge transport [5]. For example, Kong et al. introduced a single wall (SW) CNT-FET sensors using N-4-Hexafluoroisopropanolphenyl-1-pyrenebutyramide (HFIPP) receptors, achieving a detection limit as low as 50 ppb for dimethyl methylphosphonate (DMMP), demonstrating strong selectivity for volatile organic compounds [6]. In another study they demonstrated SWNT-FET sensors with p-hexafluoroisopropanol phenyl (HFIPPH) as the active material, enhancing sensitivity and selectivity for DMMP detection through efficient accumulation of DMMP vapors via hydrogen bonding [5].

The Meta-Nano-Channel Chemically-modified FET (MNChem) is a derivation of the recently introduced MNC bioFET reported for real-time, specific, and label-free sensing of biological interactions and activities [7, 8].

Estriol is a small neutral chemical molecule and is one of three markers measured during mid-gestation for the Down syndrome risk calculation and screening. Estriol secretion is increased by 1000 times during pregnancy due to placental synthesis reaching at 37 weeks onwards a concentration of 80-130 μg/ml [Mesiano et al. 256-284 (Elsevier, 2019)]. The increase in plasma estriol concentration is much higher than that of estradiol or estrone which increase only 100 times more during pregnancy [Blackburn et al. Elsevier Health Sciences, 2014]. The concentrations of free estriol in maternal plasma during pregnancy are considered as a marker for placental health, and low concentrations of multiple of median (MoM) are related to chromosomal abnormalities such as trisomy 21 and trisomy 18, fetal anomalies [Haddow et al. The New England Journal of Medicine. 327, 588-593 (1992)] and placental disorders such as preeclampsia [Shu, C. et al. Drug Des Devel Ther. 15, 2543-2550 (2021)]. Following the COVID pandemic, there is an increased demand for POC testing, that will allow the patients to perform diagnostic testing at their home, rather than in the clinics or laboratory centres.

Estriol is a member of the estrogen family. Estrogens are structurally related hydrophobic molecules, sharing a similar ring sterol backbone that is decorated with varying hydroxyl and ketone groups. Specifically, estrone (E1) has a single hydroxyl and a single ketone group, while other estrogens display only hydroxyl groups, two in estradiol (E2), three in estriol (E3), and four in estetrol (E4). Compared with small molecules, antibodies have a wider selectivity and by large present superior specificity [C. Rader et al. Trends Biotechnol. 2014, 32, 186]. Typically, flat surfaces accommodate the interaction with large protein regions, grooves enable peptide binding, and deep pockets support interactions with small molecules and haptens [R. M. MacCallum et al. J. Mol. Biol. 1996, 262. 732]. Accordingly, estrogen molecules bind hydrophobic cavities in antibodies [P. D. Jeffrey, et al, Proc. Natl. Acad. Sci. U.S.A 1993, 90, 10310].

### GENERAL DESCRIPTION

The first aspect of the present disclosure relates to a bio-transistor system comprising at least one transistor unit and a control unit. The transistor unit comprises (i) at least one channel, (ii) source and drain electrodes, (iii) at least one gate electrode and (iv) at least one active region located in proximity to the channel region and carrying at least one affinity moiety. Each affinity moiety is specific for a target molecule. The at least one active region is configured for accepting at least one sample. The transistor unit also comprises (v) at least one additional electrode positioned to be in electrical contact with the sample. The control system comprises at least one processor and memory circuitry. The control system is configured and operable for performing one or more measurements of the sample. Each measurement comprises maintaining a selected electric potential on the at least one additional electrode and determining current transmission profile through the at least one channel with respect to potential variation of the at least one gate electrode. The control unit thereby configured to determine data on the presence and/or quantity of one or more target molecules in the sample. In some embodiments, the affinity moieties of the disclosed bio-transistor are protein receptors that recognize and bind a small molecule target.

Another aspect of the present disclosure relates to a battery comprising two or more of the bio-transistor system as defined above.

Another aspect of the present disclosure related to a method for determining presence and/or quantity of at least one target molecule in at least one sample. The method comprising: (a) contacting the at least one sample with a bio-transistor having an active region carrying at least one affinity moiety, or a battery comprising at least two of the bio-transistors. It should be noted that each affinity moiety is specific for a target molecule; (b) performing one or more measurements, each measurement comprising: applying a selected electric potential on the sample, and determining current transmission profile through a channel of the bio-transistor with respect to potential variation of at least one gate electrode of the bio-transistor; and (c) processing data on the current transmission through the channel for one or more selected gate potential and one or more selected electric potential values applied on the sample and determining presence and/or quantity of the one or more target molecules in accordance with pre-stored calibration data.

Another aspect of the current disclosure is a diagnostic method for determining a physiological and/or environmental condition or state of a subject and/or a media and/or a habitat. The method comprising: (a) contacting the at least one sample with a bio-transistor having an active region carrying at least one affinity moiety, or a battery comprising at least two of said bio-transistors. Each affinity moiety is specific for a target molecule; (b) performing one or more measurements, each measurement comprising: applying a selected electric potential on the sample, and determining current transmission profile through a channel of the bio-transistor with respect to potential variation of at least one gate electrode of the bio-transistor; (c) processing data on the current transmission through the channel for one or more selected gate potential and one or more selected electric potential values applied on the sample; (d) determining presence and/or quantity of the one or more target molecules in accordance with pre-stored calibration data, thereby obtaining a target molecule value for the sample; and (e) determining that the subject and/or media and/or habitat display the physiological and/or environmental condition or state, if the at least one target molecule value obtained for the sample in step (d), is positive with respect to a reference target molecule value pre-determined for the physiological and/or environmental condition or state, or with respect to a target molecule value determined for at least one control sample.

Another aspect of the present disclosure is a screening method for identifying a compound that modulates the interaction of an affinity moiety with a target molecule in at least one sample. The method comprising: (i) contacting the at least one sample with a bio-transistor system, in the presence and the absence of at least one candidate compound. The bio-transistor having an active region carrying at least one affinity moiety, each affinity moiety is specific for a target molecule; (ii) performing one or more measurements for each sample, each measurement comprising: applying a selected electric potential on the sample, and determining current transmission profile through a channel of the bio-transistor with respect to potential variation of at least one gate electrode of the bio-transistor; and (iii) processing data on the current transmission through the channel for one or more selected gate potential and one or more selected electric potential values applied on the sample; and (iv) determining presence and/or quantity of the one or more target molecules in accordance with pre-stored calibration data, thereby determining a target molecule value for the sample in the presence of the candidate compound, and a target molecule value in the absence of the candidate compound; (v) determining that the candidate compound is a modulator of the interaction between the affinity moiety and the target molecule, if the target molecule value obtained for the sample in the presence of the candidate compound is different from the target molecule value obtained in the absence of the candidate compound.

Another aspect of the present disclosure relates to a diagnostic kit. The diagnostic kit comprising: (a) at least one bio-transistor system, and optionally, at least on of: (b) at least one control sample; and (c) at least one therapeutic agent. The bio-transistor system comprises at least one transistor unit and a control system. The transistor unit comprising: (i) at least one channel; (ii) source and drain electrodes; (iii) at least one gate electrode; (iv) at least one active region located in proximity to the channel region and carrying at least one affinity moiety, each affinity moiety is specific for a target molecule. The at least one active region is configured for accepting at least one sample; and (v) at least one additional electrode positioned to be in electrical contact with the sample. The control system comprising at least one processor and memory circuitry. The control system is configured and operable for performing one or more measurements of the sample, wherein each measurement comprises maintaining a selected electric potential on the at least one additional electrode and determining current transmission profile through the at least one channel with respect to potential variation of said the least one gate electrode. The control unit thereby configured to determine on the presence and/or quantity of one or more target molecules in the sample.

These and other aspects of the present disclosure will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. schematically exemplifies a bio-transistor system according to some embodiments of the present disclosure.

FIG. 2. schematically exemplifies a bio-transistor system formed from a plurality of transistor units according to some embodiments of the present disclosure.

FIG. 3. exemplifies method actions for determining presence and/or quantity of one or more target molecules according to some embodiments of the present disclosure.

FIG. 4A-4B. I_{DS}-V_{GL} curves for selected V_{GF} values performed for MNC biosensor biofunctionalized with HFIP receptor molecules (for PBS solution)

FIG. 4A. Illustrations of the MNChem, an optical image of the MNChem chip with electrical connections, and the employed surface functionalization strategy.

FIG. 4B. I_{DS}-V_{GL} for selected V_{GF} values and aminophenyl-HFIP-MNChem. The measurements are performed in 0.5 μL drops of 0.1 mM PBS. The data points reflect an average of 30 measurements, and the error bars are the respective standard deviations (see inset). The corresponding second derivatives are presented. The excitations of the various channels are indicated.

FIG. 5A-5B. I_{DS}-V_{GL} curves for selected V_{GF} values performed for MNC biosensor biofunctionalized with HFIP receptor molecules (for PBS solution spiked with DCNP) FIG. 5A. I_{DS}-V_{GL} for various V_{GF} values following the introduction of DCNP to aminophenyl-HFIP-MNChem. Each concentration (=drop) is measured three times, and the presented data are the corresponding averages and standard deviations (error bars).

FIG. 5B. Control non-specific measurements for: 1) DCNP introduction to an unmodified MNChem, 2) DCNP introduction to APTMS-modified MNChem, 3) Methanol (MeOH) introduction to aminophenyl-HFIP-MNChem, and 4) tryptophan introduction to aminophenyl-HFIP-MNChem. The selected target concentrations are provided in the legend. The data points and the error bars follow the convention in FIG. 5A.

FIG. 6. Extracted calibration curves

Calibration curves reflecting specific and label-free sensing of DCNP. Illustrations of the relevant channel configurations are shown as well.

FIG. 7. Performance of DCNP sensing

The figure presents Table 1 that shows a summary for the sensing performance of the proposed aminophenyl-HFIP-MNChem towards DCNP, specifically, sensing of a small molecule using a receptor molecule.

FIG. 8A-8B. I_{DS}-V_{GL} curves for selected V_{GF} values performed for MNC biosensor biofunctionalized with anti-estriol antibodies (for diluted plasma)

FIG. 8A. An optical image of the MNC biochip, a 3D illustration of the MNC bioFET, and a cross-section of the MNC bioFET midway between source and drain.

FIG. 8B. I_{DS}-V_{GL} curves for selected V_{GF} values performed with 0.5 μL drops of 1:100 diluted plasma. The MNC biosensor is unmodified. Each data point is an average of 51 measurements (17 drops, each drop is measured 3 times), and the error bars are the corresponding standard deviations (see inset).

FIG. 9A-9D. I_{DS}-V_{GL} curves for selected V_{GF} values performed for unmodified MNC BioFET and MNC BioFET biofunctionalized with anti-estriol antibodies

FIG. 9A. An illustration showing the process of Si/SiO₂ surface biofunctionalization.

FIG. 9B. Contact angle and ellipsometry measurements of Si/SiO₂ samples post various modification steps.

FIG. 9C. EIS measurements for the various modification steps.

FIG. 9D. I_{DS}-V_{GL} curves for various V_{GF} values for unmodified MNC BioFET and MNC BioFET biofunctionalized with anti-estriol antibodies. The data points and the error bars (see inset) reflect the averages and standard deviations of 16 measurements (4 drops each measured 4 times).

FIG. 10A-10E. I_{DS}-V_{GL} curves for selected V_{GF} values performed for MNC BioFET biofunctionalized with anti-estriol antibodies

FIG. 10A. I_{DS}-V_{GL} for selected V_{GF} values for MNC bioFET modified with anti-estriol antibodies. The measurements are performed in 0.5 μL drops of 1:100 diluted plasma. The data points reflect an average of 51 measurements (Total of 17 drops each measured 3 times), and the error bars are the respective standard deviations (see inset).

FIG. 10B. The corresponding second derivatives. The excitations of the various channels are indicated.

FIG. 10C. Left: I_{DS}-V_{GL} for V_{GF}=0.5 V for 0.5 μL drops of 1:100 diluted plasma spiked with estriol concentration from 1 fg/ml-10 μg/ml. The sensing area of the MNC bioFET is biofunctionalized with anti-estriol antibodies. The I_{DS}-V_{GL} curves are presented on both linear and logarithmic y-axis scales. Each data point is the average of 4 measurements performed for each drop, and the error bars are the corresponding standard deviations (see inset). Right: the respective I_{Normalized} curves. The channel configuration is also indicated.

FIG. 10D. Same as FIG. 10C only for V_{GF}=−0.5 V.

FIG. 10E. Same as FIG. 10C only for V_{GF}=−1.5 V.

FIG. 11A-11E. I_{Normalized} curves (controls)

FIG. 11A. I_{Normalized} curves for estriol introduced to an unmodified bioFET.

FIG. 11B. I_{Normalized} curves for estriol introduced to a MNC bioFET modified with APTMS.

FIG. 11C. Estrone molecules introduced to MNC bioFET modified with anti-estriol antibodies.

FIG. 11D. Estradiol molecules introduced to an MNC bioFET modified with anti-estriol antibodies.

FIG. 11E. I_{Normalized} curves for the successive application of estrone, estradiol and estriol drops.

All measurements presented in FIG. 11 were performed in 0.5 μL drops of 1:100 diluted plasma.

FIG. 12. Calibration curves of I_{Normalized} vs. estriol concentration

Calibration curves of I_{Normalized} vs. estriol concentration presenting specific and label-free estriol sensing with the MNC bioFET. The illustrations on the right reflect the corresponding channel configuration.

FIG. 13. The proposed sensing mechanism

The figure shows an illustration of the suggested sensing mechanism.

FIG. 14. Performance of the estriol sensing

The figure presents Table 2 that show the dependency of the sensitivity, linearity, dynamic range and LOD on channel configuration for the specific and label-free sensing of estriol in 0.5 μL drops of 1:100 diluted plasma with the MNC bioFET.

FIG. 15. I_{DS}-V_{GL} curves for selected V_{GF} values measured for MNC biosensor biofunctionalized with anti-AFP antibodies (background measurement)

I_{DS}-V_{GL} curves for selected V_{GF} values measured for MNC biosensor biofunctionalized with anti-AFP antibodies. Each data point is an average of 64 measurements (16 drops, each drop is measured 4 times), and the error bars are the corresponding standard deviations (see inset).

FIG. 16A-16D. Process of Si/SiO₂ surface biofunctionalization with anti-AFP antibodies

FIG. 16A. An illustration showing the process of Si/SiO₂ surface biofunctionalization. The corresponding contact angle measurements are also shown.

FIG. 16B. Ellipsometry measurements and contact angle measurements of Si/SiO₂ samples post various modification steps.

FIG. 16C. EIS measurements showing the real and imaginary capacitances for the various modification steps.

FIG. 16D. I_{DS}-V_{GL} for various V_{GF} values for unmodified MNC device and biofunctionalized MNC biosensor. The data points and the error bars (see inset) reflect the averages and standard deviations of 12 measurements (3 drops each measured 4 times).

FIG. 17A-17C. I_{DS}-V_{GL} for selected V_{GF} values for MNC biosensor modified with anti-AFP molecules

FIG. 17A. I_{DS}-V_{GL} for selected V_{GF} values for MNC biosensor modified with anti-AFP molecules. The measurements are performed in 1:100 diluted serum. The data points reflect an average of 42 measurements (Total of 14 drops each measured 3 times), and the error bars are the respective standard deviations (see inset).

FIG. 17B. The second derivatives of the curves presented in FIG. 17A where a peak reflects the excitation of a conducting channel. The dependence of channel excitation on gates' voltage configuration is shown.

FIG. 17C(i)-17C(iv). Non-specific measurements for: AFP introduced to an unmodified MNC biosensor (FIG. 17C(i)), AFP introduced to an MNC biosensor modified with APTMS (FIG. 17C(ii)), hCG molecules introduced to an MNC biosensor modified with anti-AFP antibodies (FIG. 17C(iii)), and PSA molecules introduced to an MNC biosensor modified with anti-AFP antibodies (FIG. 17C(iv)). All measurements are performed in 1:100 diluted serum. The insets present magnifications showing the error bars reflecting the standard deviations of the measured populations.

FIG. 18A-18C. I_{DS}-V_{GL} for selected V_{GF} values for different concentrations of AFP molecules

FIG. 18A. I_{DS}-V_{GL} for selected V_{GF} values for 10 concentrations of AFP molecules. The measurements are performed in 1:100 diluted serum. Each concentration (=drop) is measured 4 times. The insets show the error bars reflecting the standard deviations. The non-specific signals are accounted for and removed.

FIG. 18B. The extracted Readout corresponding to the curves presented in FIG. 18A. The insets show the corresponding error bars. The labels at the top of the graphs indicate the corresponding conducting channels.

FIG. 18C. An illustration showing one possible mechanism for the Readout polarity switch induced by the double layer electric field.

FIG. 19A-19B. Readout calibration curves for AFP molecules

FIG. 19A. Readout calibration curves. The illustrations on the right reflect the channel configurations. The vertical dashed grey lines indicate the calibration threshold.

FIG. 19B(i)-19B(ii). The dependency of the calibration threshold on V_{GF} (FIG. 19B(i)). The non-dependency of the calibration threshold on V_{GL} (FIG. 19B(ii)). The shift between the lines for V_{GL}=0, −0.5, −1 and −1.5 V are only for the purpose of visualization.

FIG. 20. AFP sensing performance

The figure presents Table 3, that shows a summary of the MNC biosensor sensing performance for specific and label-free detection of AFP.

### DETAILED DESCRIPTION OF EMBODIMENTS

The present disclosure provides bio-transistor systems, methods and kits thereof, for effective detection of a target molecule in a sample, using an interaction based on the affinity between small molecules and proteineous molecules. More specifically, the present disclosure effectively demonstrates bio-transistor systems and methods for detecting small molecule targets using proteineous affinity moieties, for example, antibodies or receptors. The diagnostic bio-transistor systems and method provided herein demonstrate effective detection of synthetic small molecules (e.g., organophosphates), and natural small molecules (e.g., steroid molecules such as estriol) that serves as a marker for placental health, in environmental samples, as well as in biological samples, specifically, blood and serum. As a proof of concept (POC), the current disclosure employs an MNChem chemically modified with a recognition layer composed of 2-(4-Aminophenyl)-1,1,1,3,3,3-hexafluoro-2-propanol (aminophenyl-HFIP) receptors for specific and label-free sensing of the Organophosphate (OP) simulant Diethyl cyanophosphonate (DCNP). The hydrogen-bonding interaction facilitated by HFIP substituents enables the effective adsorption of organophosphorus solutions or vapors [Barlow, J. W. et al. doi: 10.1002/pen.760271003]. This allows the utilization of organic compounds containing HFIP groups as sensitive sensors for various applications [Grate, J. W. et al. (2008) doi: 10.1021/cr068109y]. As a receptor molecule, HFIP forms robust hydrogen bonds with target analytes, enhancing their selective capture and interaction with the sensor surface [Amara, et al. Macromolecules 38, (2005)]. Additionally, HFIP's compatibility with sensor fabrication processes and its ability to covalently bind onto sensor surface make it a preferred choice for enhancing sensor performance in OP detection applications [Amara, et al. Macromolecules 38, (2005)]. Importantly, the optimization of hydrogen-bond acidity and basicity through the electron-withdrawing effect of fluorine atoms in HFIP efficiently prevents self-association of HFIP substituents [Grate, J. W., et al. Anal. Chem. 71, (1999)]. This prevents the HFIP from clustering or aggregating, which could interfere with their binding functionality. The selected target molecule, DCNP, is commonly used as a simulant for OP pesticides and nerve agents in research and testing due to its structural similarity and relative ease of handling compared to actual nerve agents like Sarin or venomous agent X (VX).

The MNC platform aims to address the inherent challenge of local gating induced by the biological or chemical interactions distributed non-uniformly at the device sensing area. This is in stark contrast with the global gating provided by conventional metal-gate transistors. The MNC method allows an electrostatic tuning of drain-source current (I_{DS}) such as to optimally couple I_{DS} electrodynamics with the electrostatics of the specific interactions at the sensing area [7]. The MNChem sensor is fabricated in a CMOS process which ensures optimal electrical performance in terms of noise and amplification. The current work employs the MNChem sensor, modified with an aminophenyl-HFIP recognition layer, for real-time label-free and specific detection of DCNP in 0.5 μL drops of 0.1 mM PBS pH7.4. Calibration curves, characterization of surface modification, and extensive control measurements are provided.

As a POC, the current work also demonstrates sensing of estriol, a small neutral molecule, with the recently introduced meta-nano-channel (MNC) bioFET. Estriol is one of three markers measured during mid-gestation for the Down syndrome risk calculation and screening. Estriol secretion is increased by 1000 times during pregnancy due to placental synthesis reaching at 37 weeks onwards a concentration of 80-130 μg/ml. The increase in plasma estriol concentration is much higher than that of estradiol or estrone which increase only 100 times more during pregnancy. The concentrations of free estriol in maternal plasma during pregnancy are considered as a marker for placental health, and low concentrations of multiple of median are related to chromosomal abnormalities such as trisomy 21 and trisomy 18, fetal anomalies and placental disorders such as preeclampsia. Following the COVID pandemic, there is an increased demand for POC testing, that will allow the patients to perform diagnostic testing at their home, rather than in the clinics or laboratory centres. As such, estriol along with beta HCG and alfa fetoprotein can be an excellent POC test, that if available, will increase maternal compliance for aneuploidy mid-gestation screening and as a surrogate marker for potential placental complications.

The MNC bioFET provides a tunable channel configuration to enable optimal coupling between the electrostatics of the biological interactions and the electrodynamics of the conducting readout current [7]. The MNC biosensor is fabricated in a CMOS process which entails high-end bioFETs operating at the thermodynamic limit in terms of noise levels and amplification and coupled with low-cost and low power. Moreover, the ultimate miniaturization permitted by current microelectronic industry paves the way to unparalleled multiplexing in ultra-small samples.

The recognition layer is composed of anti-estriol antibodies to provide high specificity toward estriol compared with the other members of the estrogen family. Antibody-antigen binding is non-covalent and is mediated by complementarity in the binding interface charge and shape. Typically, flat surfaces accommodate the interaction with large protein regions, grooves enable peptide binding, and deep pockets support interactions with small molecules and haptens. In the present disclosure the inventors employ the MNC bioFET to the specific and label-free sensing of estriol from ultra-small samples of 0.5 μL drops of 1:100 diluted plasma. Despite the small size and the electrical neutrality of estriol, real-time is demonstrated, specific, and label-free sensing of estriol with excellent linearity, sensitivity, dynamic range extending to ten orders of magnitude in estriol concentration and limit-of-detection of 1 fg mL⁻¹. Extensive control and non-specific measurements are reported including the response to other neutral members of the estrogen family.

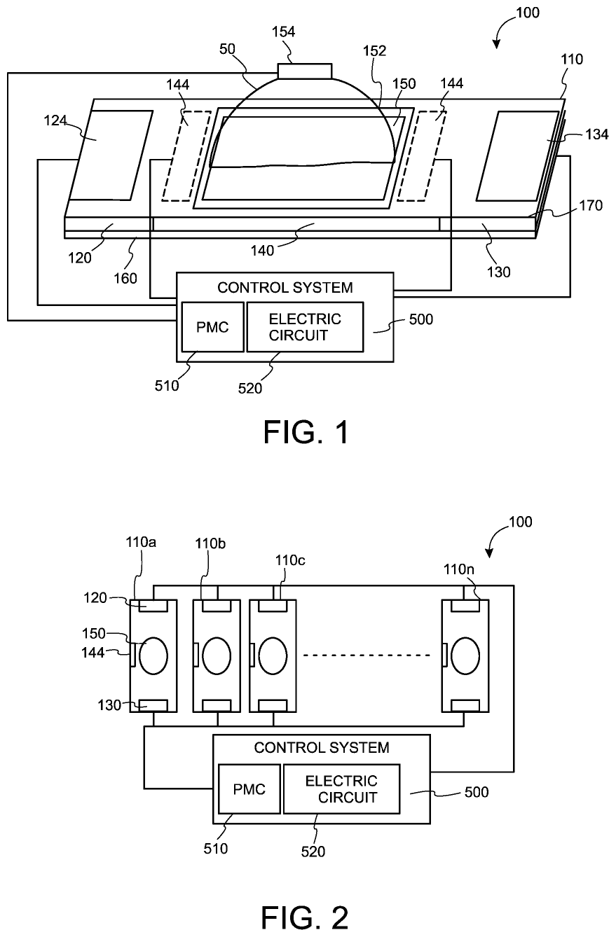

As indicated above, the present disclosure provides a bio-transistor system configured and operable for detection of presence and/or quantity of one or more selected molecules. An exemplary configuration of bio-transistor system 100 is illustrated in FIG. 1. The system includes at least one transistor unit 110 including source 120 and drain 130 regions associated with respective source 124 and drain 134 electrodes, and a channel region 140 between them. The transistor unit 110 also includes one or more gate electrodes 144, located and configured to apply selected electric field on the channel 140 region to thereby enable switching of the transistor unit 110 by affecting charge carriers within the channel 140. Typically, as exemplified herein the source 120, drain 130 and channel 140 regions are placed on a back insulator layer 160 and covered by a top insulator 170. Further, in some embodiments, the at least one gate electrode may be a side (e.g., lateral) gate electrode with respect to the channel region 140, alternatively or additionally, the at least one gate electrode may be positioned between one of the sources and drain electrodes 124 and 134 and the active region 150. The active region 150 is located on top of the channel region 140, electrically insulated from the channel region 140 by the top insulator 170. The at least one gate electrode 144 is generally separated from the channel region 140 by a gate insulator, which may be a portion of top insulator 170. In some configurations, the transistor unit 110 may be formed of one or more semiconductor materials such as silicon, having selected doped regions with selected p-type or n-type doping. For example, the active layer defining the source 120 and drain 130 regions and the channel region 140 between them may be formed of silicon semiconductor having varying p- and n-type doping regions.

The transistor unit 110 further includes an active region 150 located in vicinity (e.g., above) a region of the channel 140 and separated from the channel, e.g., by top insulator 170. The active region 150 is formed of a region of top insulator 170, which is modified to carry selected one or more types of binding molecules, or affinity moieties, selected to bind one or more selected target molecules. This configuration provides the bio-transistor system 100 with capabilities for detection of the selected target molecules. The transistor unit 110 is configured to accept a liquid sample 50, e.g., a liquid drop, positioned on, or in contact with, the active region 150. In some configurations, the active region 150 may be surrounded by edge 152 configured to limit flow of liquid sample and prevent contact of the sample with electrodes of the unit 110 such as gate electrodes 144. The edge 152 may be formed of any electrically insulating material and may be a part of top insulator 170. Transistor unit 110 may also include at least one additional electrode 154 positioned to be in electric contact with the liquid sample 50, the additional electrode 154 is also referred to as quasi-reference electrode or sample electrode.

As indicated above, transistor unit 110 may be formed of silicone including n- and p-doped silicon regions forming the channel region 140 as well as source 120 and drain 130 regions. Top 170 and bottom 160 insulators may be formed of silicon oxide layers. It should be noted that in some other embodiments, the transistor may be formed of selected one or more other semiconductor materials as the case may be.

Bio-transistor system 100 may also include a control system 500. Control system 500 may include one or more processor and memory circuitries (PMC) 510 and may also include an electric circuit 52 configured to selectively apply electric potential and/or transmit electric current through the electrodes of transistor unit 110 to enable bio-transistor system 100 to determine data on presence and/or quantity of target molecules in the sample 50.

In this connection PMC 510 may include one or more processors and memory, and may include, or be associated with, an I/O interface for receiving input data and for transmitting output instructions and/or data for operation of the bio-transistor system 100. PMC 510 is operatively connected to the I/O interface and is configured to provide processing necessary for operation of the bio-transistor as described herein. PMC 510 is configured to execute one or more functional operations in accordance with computer readable instructions implemented on a computer readable medium (e.g., non-transitory memory) being pre-stored at the memory of PMC 510 or at a separate computer readable medium.

In this connection, PMC 510 may operate the electric circuit 520 for selectively providing electric potential to the electrodes of transistor unit 110, and for determining data on current flow between the source electrode 124 and drain electrode 134. In this connection the control unit 500 is configured and operable for performing one or more measurements of the sample 50 to determine data on presence and/or quantity of target molecules in the sample 50.

More specifically, in some embodiments, each of the one or more measurements may include operation of the control system to maintain a selected electric potential V_{GF} on electrode 154 to maintain a selected potential on the sample 50. Additionally, while maintaining a selected potential V_{GF} on the sample, the control system 500 may operate for determining current transmission profile between the source 124 and drain 134 electrodes with respect to varying potential V_{G} on at least one gate electrode 144. For example, this may be done by applying a selected potential between the source 124 and drain 134 electrodes and determining level of current transmission between the source 120 and drain 130 regions through the channel 140 with respect to potential V_{G} applied to the at least one gate electrode 144. Typically, each of the one or more measurements may be performed using a different selected potential V_{GF} applied on the sample 50.

As described below, the present disclosure is based on the inventors' understanding that interactions between target molecules and selected affinity moieties located on the active region 150 of the transistor unit 110 affect the transistor source-gate transmission in certain conditions. The interactions between target molecules and affinity moieties causes variation in distribution of electric changes resulting in variation of electric fields around the active region 150, which also affects the channel region 140. Accordingly, each measurement may include collecting data on current to gate potential (I-V_{G}) characteristics of the transistor unit 110 for given sample potential V_{GF}.

Typically, the control system 500 may be configured to perform a selected number of one or more, or two or more measurements, using different sample potentials V_{GF}, and to store collected data on current for different gate potential values for each measurement. This measurement data, indicative of transistor unit 110 I-V_{G} characteristics for one or more different sample potential values is indicative of presence and/or quantity of the target molecules in the sample 50.

To this end, the control system 500 may include pre-stored data, e.g., pre-stored at the memory of PMC 510, or stored in a remote location accessible view network communication, for calibrating transistor unit 110 characteristics to data on presence and/or quantity of target molecules in the sample 50. The pre stored data may be indicative of I-V_{G} characteristics of the transistor unit 110 as a function of presence and/or quantity of the target molecules for one or more given sample potentials V_{GF}. Additionally, or alternatively, the pre stored data may be indicative of a relation between current transmission through the channel as a function of sample potential V_{GF}, for one or more given gate voltage V_{G} values. After determining data on presence and/or quantity of the target molecules, the control system 500 may operate to generate output signal indicative of the data and provide the output signal via I/O interface to be presented to an operator using user interface and/or for use in further processing.

As indicated above, the bio-transistor system 100 utilizes at least one affinity moiety (also referred to herein as selected affinity moieties) placed (adsorbed, attached) on an active region 150 located in vicinity to the channel region 140. The active region 150 may be separated from semiconductor region of the channel region 140 by an electric insulating layer 170, such that electrostatic changes at the active region 150 may apply electric fields affecting the channel region 140.

Selection of the affinity moieties determine one or more target molecules to be identified using the bio-transistor system 100. To enable detection of a plurality of different target molecules, the present disclosure may also provide a bio-transistor system 100 including a plurality of transistor units, each carrying a selected different type of affinity moieties, selected to interact with one or more target molecules, including e.g., one or more different target molecules. It should be understood that in some embodiments, each of the affinity moieties is specific for one target molecule. Thus, in some embodiments the disclosed bio transistor system may comprise at least one affinity moiety, and in some embodiments, a plurality of affinity moieties that are specific for one or more target molecule. Each of the affinity moieties is specific for one target molecule, but for each target molecule it is possible to have more than one either identical or different affinity moiety. In yet some further embodiments, the disclosed bio transistor system may comprise different affinity moieties specific for more than one target molecule, wherein each of the affinity moiety is specific for one target molecule. In this connection reference is made to FIG. 2 exemplifying a bio-transistor array configuration of system 100. The system includes a selected number of transistor units 110a to 110n, each having source and drain regions 120 and 130 and a channel region between them, and active region 150 carrying the selected affinity moieties, gate electrode 144, and an additional electrode (not shown) positioned to be in contact with a sample positioned on the active region 150. The plurality of transistor units 110a-110n are connected to a control system 500 and are independently operated for determining data on presence and/or quantity of the respective target molecules as described herein.

In some embodiments, the bio-transistor system 100 may include a sample channel configured for receiving a liquid sample and channeling portions of the liquid samples to different active regions of a plurality of transistor units as illustrated in FIG. 2. To avoid variation in electric potential between the sample portions located on the different active regions, the channel may include a selected number of electrical insulating valves configured to close prior to performing measurements on the samples.

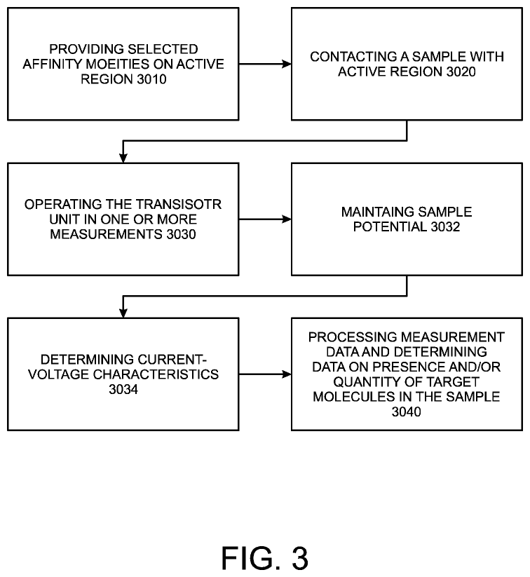

FIG. 3 exemplifies a method for determining presence and/or quantity of at least one type of target molecules according to some embodiments of the present disclosure in a way of a block diagram. As shown, the method includes providing selected affinity moieties in contact (e.g., adsorbed, attached) with an active region 3010 of one or more transistor units. The bio-transistor unit may be treated with the selected affinity moieties during production and provided for use in determining presence and/or quantity of the selected target molecules. To determine presence and/or quantity of at least one type of target molecules, the method includes contacting a sample with the active region of the one or more transistor units 3020 and operating the transistor unit to perform one or more measurements 3030. Generally, each measurement includes maintaining a sample potential 3032, using one or more additional electrodes being in electric contact with the sample, and determining current-voltage characteristics of the transistor unit 3034. As indicated, the method may include a selected number of measurements, typically using different one or more potentials applied to the sample. Following one or more (or two or more) measurements, the method includes processing the measurement data and determining data on presence and/or quantity of the target molecules 3040. The processing may generally include using pre-provided calibration data indicative of variation in transistor current-voltage characteristics for given amounts of the target molecules in the sample.

Accordingly, the system and method of the present disclosure may utilize predetermined calibration data, determined for given activity region 150 carrying given amount of affinity moieties of selected type. In some examples the calibration data may be in the form of a multi-dimensional calibration data including data on variation of the source-drain current with respect to different gate potentials V_{G} and to different sample potentials V_{GF}. Accordingly, ss indicated above, variation of the sample potential V_{GF} may be used as a parameter for determining presence and/or quantity of the target molecules. In some other embodiments, variation of the sample potential V_{GF} may provide for varying detection range for difference ranges of quantity of the target molecule, where for each value of sample potential, variation of gate potential V_{G} and the relation between gate potential and source-drain current provides the calibration for quantity of target molecules in the sample.

Referring back to FIG. 3, the method of the present disclosure utilizes performing one or more measurements of the sample. As indicated above, each measurement includes maintaining sample potential V_{GF} at a selected value and determining source-drain current for selected range of gate potentials V_{G}. Typically, the different measurements utilize different values of the sample potential V_{GF}. It should be understood that the measurement may also utilize a selected, generally constant, source-drain voltage to eliminate, or at least significantly reduce effects of the potential variation on the interaction between the affinity moieties and target molecules. Additionally, variation of sample potential V_{GF} may affect concentration of the target molecules within the sample, e.g., forming layers of different concentrations of the target molecules, thereby limiting detection accuracy. To solve this issue, the present technique may utilize a selected number of different sample potential V_{GF} values, and respective calibration data.

Accordingly, the system and method of the present disclosure provide for determining presence and/or quantity of selected target molecules within a sample. As described above, the present technique utilizes calibration data, determined in accordance with known concentrations of the target molecules. Typically, the calibration data may be determined in accordance with amount and/or density of affinity moieties associated with the active region 150.

In accordance with structure of the transistor unit 110, variation in source-drain current, associated with gate potential V_{G}, and sample potential V_{GF}, may be associated with concentration of charge carrier in the channel region 140. In some configurations of the transistor unit 110, variation of gate potential V_{G} may affect size and/or shape of the channel region 140 and accordingly affect current transmission through the channel. An exemplary structure of transistor unit suitable for affecting channel shape and dimensions by variation of gate potential V_{G} is described in US 2023/0022648 incorporated herein for reference with respect to configurations of the transistor unit [2].

Thus, the first aspect of the present disclosure relates to a bio-transistor system comprising at least one transistor unit and a control unit. The transistor unit comprises (i) at least one channel, (ii) source and drain electrodes, (iii) at least one gate electrode and (iv) at least one active region located in proximity to the channel region and carrying at least one affinity moiety (also referred to herein as selected affinity moieties). It should be noted that each affinity moiety is specific for a target molecule. It should be noted that each affinity moiety is specific for a target molecule. Specifically, each of the affinity moieties is specific for one target molecule. In yet some further embodiments, the active region may carry two or more different affinity moieties, each specific for one target molecule. In some embodiments, different affinity moieties may be specific for the same target molecule. Alternatively, different affinity moieties may be specific for different target molecules, wherein each of the affinity moieties is specific for one target molecule. The at least one active region is configured for accepting at least one sample. The transistor unit also comprises (v) at least one additional electrode positioned to be in electrical contact with the sample (e.g., a liquid sample). The control system comprises at least one processor and memory circuitry. The control system is configured and operable for performing one or more measurements of the sample. Each measurement comprises maintaining a selected electric potential on the at least one additional electrode and determining current transmission profile through the at least one channel with respect to potential variation of the at least one gate electrode. The control unit thereby configured to determine data on the presence and/or quantity of the at least one target molecules in the sample.

In some embodiments of the present disclosure, the control system is configured to performed two or more measurements utilizing two or more different selected electric potentials applied to the at least one additional electrode.

In some other embodiments, the control system comprises pre-stored calibration data comprising data on electric transmission through the channel for given gate electrode potential with respect to one or more selected electric potentials applied to the at least one additional electrode.

In some further embodiments, the control system comprises pre-stored calibration data comprising data on electric transmission trough the channel with respect to variation of the gate electrode potential.

It should be noted that the pre-stored calibration data may be generated from different concentrations of the target molecule in a sample. More specifically, the pre-stored calibration data may be generated from at least one concentration of target molecule. Still further, the pre-stored calibration data may be generated from at least 1, 2, 3, 4, 5, 6, 7, 8, 9, 10, 20, 30, 40, 50, 60, 70, 80, 90, 100, 1000 or more different concentrations of the target molecule in the sample. It should be noted that different concentrations of the target molecule may be also affected from the different dilutions of the samples.

In some embodiments of the present disclosure, the bio-transistor system comprising a plurality of two or more transistor units comprising respective plurality of two or more active regions carrying two or more different or identical types of affinity moieties.

In some other embodiments, the active region is separated from the channel region by an electrical insulator layer.

In some further embodiments, the at least one gate electrode is electrically insulated from the active region.

Still further, in some embodiments, the control system comprises at least one electrical circuit coupled vie electrical connection to the transistor unit and configured to provide selected electric potentials to electrodes of the transistor unit.

In some embodiments of the present disclosure, the at least one affinity moiety comprises at least one of: an amino acid-based molecule, a nucleic acid-based molecule, a small molecule, a carbohydrate-based molecule, a lipid-based molecule or any combination thereof. The affinity moiety specifically binds, either directly or indirectly, the at least one target molecule in the sample.

In some further embodiments, the affinity moiety comprises, or is derived from a component of an affinity pair. The term “affinity moiety” or “affinity molecule” as used herein, or “affinity pair” (as used herein after), refers to a corresponding binding couple biomolecule partners. The term “Affinity” or “binding affinity” is the strength of the binding interaction between a binding biomolecule to its binding partner or target (e.g., a protein, DNA or small molecule). Binding affinity is typically measured and reported by the equilibrium dissociation constant (KD), which is used to evaluate and rank order strengths of bimolecular interactions. The smaller the KD value, the greater the binding affinity of the binding molecule for its target. The larger the KD value, the weaker the target molecule and affinity molecule are attracted to and bind to one another. Binding affinity is influenced by non-covalent intermolecular interactions such as hydrogen bonding, electrostatic interactions, and hydrophobic and van der Waals forces between the two molecules. In addition, binding affinity between a binding molecule and its target molecule may be affected by the presence of other molecules. The affinity pair comprises at least one of: receptor-ligand, antibody-antigen, enzyme-substrate, aptamer-aptamer target, or any combination thereof. It should be noted that each affinity moiety is specific for one target molecule. In some embodiments, the affinity moiety and the target molecule recognized by the affinity moiety form together an affinity pair. The term “specific” and “specifically bind” is as described herein after.

It should be understood that the affinity molecule specifically binds the at least one target molecule in the sample.

In some embodiments, the target molecule comprises at least one of: an amino acid-based molecule, a nucleic acid-based molecule, a small molecule, a carbohydrate-based molecule, a lipid-based molecule or any combination thereof.

As indicated above, the affinity molecule, and/or the target molecule and/or the candidate compound disclosed herein after in connection with the screening methods, may be amino acid-based, a nucleic acid-based, a small molecule-based, a carbohydrate-based and/or a lipid-based. The term “-based” as used herein refers to the at least one affinity moiety or molecule, and/or to the target molecule, which is composed of the building block specified prior to “-based”. For example, amino acid-based affinity molecule/s and/or target molecule/s, are affinity molecule/s and/or target molecule/s, which are composed of amino acids, such as proteins, peptides, glycoproteins, lipoproteins, etc. Still further, nucleic acid-based affinity molecules and/or target molecule/s, are affinity molecule/s and/or target molecule/s, which are composed of nucleic acids such as polynucleotides, aptamers, etc. Small molecule-based affinity molecule/s and/or target molecule/s are affinity molecule/s and/or target molecule/s, which are composed of small molecules, which are low molecular weight organic compound, having a molecular weight lower than 900 Daltons. Carbohydrate-based affinity molecules are affinity molecules which are composed of carbohydrates such as monosaccharides, disaccharides, oligosaccharides, and polysaccharide as well as proteoglycans, glycoproteins, etc. lipid-based molecules are affinity molecule/s and/or target molecule/s, which are composed of lipids such as fats, waxes, sterols, fat-soluble vitamins (such as vitamins A, D, E and K), monoglycerides, diglycerides, phospholipids, proteolipids, and others.

As indicated above, in some embodiments the at least one affinity moiety and/or the at least one target molecule of the disclosed bio transistor systems and methods, may be, or may comprise a small molecule. A small molecule in the context of the present disclosure refers to a low molecular weight organic compound, having a molecular weight lower than 900 Daltons, and in some embodiments less than about 2 kilodaltons (kDa) in mass. In some embodiments, the small molecule is less than about 1.5 kDa, or less than about 1 kDa. In some embodiments, the small molecule is less than about 900 daltons (Da), 800 Da 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In accordance with the present disclosure, when referring to a small molecule it includes also crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” or “polymorph,” as used herein include all crystalline and amorphous forms of a small molecule, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

In accordance with the present disclosure, the term “small molecule” may include pharmaceutically acceptable forms of the recited compounds, including chelates, non-covalent complexes, prodrugs, and mixtures thereof. Further and in accordance with the preset disclosure, the term “small molecule” includes also pharmaceutically acceptable forms of a particular molecule and as such the term small molecule also encompasses pharmaceutically acceptable salts.

As indicated above, in some embodiments the at least one affinity moiety and/or the at least one target molecule of the disclosed bio transistor systems and methods, may be, or may comprise an amino acid-based molecule. An “amino acid-based molecule”, as used herein refers to a molecule that is primarily composed of amino acids or derivatives of amino acids. Amino acids are organic compounds that contain both an amino group (—NH2) and a carboxyl group (—COOH), along with a unique side chain specific to each amino acid. Amino-acid based molecules in accordance with the present disclosure encompass various structures including peptides, that are short chains of amino acids linked by peptide bonds (typically consist of 2 to 50 amino acids), polypeptides, that are intermediate-length chains of amino acids that can fold into specific structures and may function independently or as part of a larger protein complex and proteins, that are composed of long chains of amino acids, typically consisting of 50 or more amino acids, and fold into specific three-dimensional structures that determine their function in biological processes. An example for an affinity moiety-target pair composed of a protein include, but is not limited to the antibody-antigen, receptor-ligand and peptide aptamers and targets thereof. Still further, it should be understood that amino-acid-based molecule as used in the present disclosure further encompasses amino acid derivatives, specifically, molecules derived from amino acids through chemical modifications.

Still further, in some embodiments, the at least one affinity moiety and/or the at least one target molecule of the disclosed bio transistor systems and methods, may be, or may comprise a lipid-based molecule.

A lipid-based molecule, as used herein, is a chemical compound primarily composed of lipids, which are a diverse group of hydrophobic or amphipathic organic molecules. Lipids are insoluble in water but soluble in nonpolar solvents. It should be understood that the present disclosure encompasses any affinity moiety and/or target molecule composed of or comprising a lipid-based molecule, of any type, for example, triglycerides, that consist of three fatty acids attached to a glycerol backbone, phospholipids, that have two fatty acids and a phosphate group attached to glycerol, steroids, that have a structure based on a carbon skeleton with four fused rings (e.g., Cholesterol), glycolipids, that contain a carbohydrate group attached to a lipid, and fatty Acids, that are carboxylic acids with a long hydrocarbon chain, which can be saturated (no double bonds) or unsaturated (one or more double bonds).

In yet some further embodiments, the at least one affinity moiety and/or the at least one target molecule of the disclosed bio transistor systems and methods, may be, or may comprise a carbohydrate-based molecule. A carbohydrate-based molecule, as used herein, is a chemical compound that primarily consists of carbohydrate components, which are organic molecules made up of carbon (C), hydrogen (H), and oxygen (O) atoms, usually with the hydrogen and oxygen atoms in a ratio of 2:1, as in water. Carbohydrates are one of the four main classes of biomolecules and can be simple sugars (monosaccharides like glucose and fructose), double sugars (disaccharides like sucrose and lactose), or complex carbohydrates (polysaccharides like starch, cellulose, and glycogen).

In some alternative embodiments the at least one affinity moiety and/or the at least one target molecule of the disclosed bio transistor systems and methods, may be, or may comprise a nucleic acid molecule. The term nucleic acid molecule as used herein relates to a complex organic substance that forms the basis of genetic material in all living organisms. It consists of long chains of nucleotides, that form the basic units of nucleic acids, each nucleotide is composed of three components: a phosphate group, a five-carbon sugar (either ribose in RNA or deoxyribose in DNA), and a nitrogenous base. There are four main types of nitrogenous bases in DNA (adenine [A], thymine [T], cytosine [C], and guanine [G]) and in RNA (adenine [A], uracil [U], cytosine [C], and guanine [G]). The nucleotides are linked together by covalent bonds between the phosphate group of one nucleotide and the sugar of the next, forming a sugar-phosphate backbone with protruding nitrogenous bases. The present disclosure encompasses Deoxyribonucleic Acid (DNA) molecules, Ribonucleic Acid (RNA), any combinations thereof or any derivatives thereof. It should be understood that the target molecule is specifically recognized and bound by the affinity moiety.

In some further embodiments, the target molecule comprises, or is derived from a component of an affinity pair. The affinity pair comprises at least one of: receptor-ligand, antibody—an antigen, enzyme-substrate, aptamer-aptamer target, or any combination thereof.

It should be understood that the target molecule specifically interacts with the affinity moiety. The term “target molecule” as used herein refers to at least one specific molecule in a sample for which determining its presence and/or quantity is desired. This target molecule will be the corresponding partner of the affinity moiety. It should be understood that a target molecule as used herein, further encompasses a single molecule or alternatively two or more molecules, subunits, complexes, conjugates and the like, forming a specific recognition partner recognized and bound by the specific affinity moiety of the disclosed bio-transistor systems.

The present bio transistor systems and the detection methods disclosed herein are based on the specific recognition and binding of the at least one affinity moiety and the target molecule, that form together an affinity pair.

In some embodiments, the affinity pair in accordance with the present disclosure may comprise at least one amino acid-based molecule and at least one small molecule thereby forming together an affinity pair. In some embodiments, the affinity moiety may be or may comprise an amino acid-based molecule. In yet some further embodiments, the target molecule may be or may comprise at least one small molecule. Affinity pair the comprise amino acid-based and small molecule components may be in accordance with some embodiments an antibody-antigen pair, specifically, when the antigen is a small molecule, forming the antigen recognized by the specific antibody. In yet some further embodiments an optional affinity pair may be a receptor-ligand affinity pair. Other affinity pairs, specifically, enzyme-substrate, aptamer-aptamer target, or any combination thereof, are also optional in accordance with the present disclosure.

More specifically, in some embodiments, the target molecule is a ligand composed of a small molecule and the corresponding receptor for such ligand is an amino acid-based receptor serving as the affinity moiety. More specifically, in some embodiments, an affinity pair, for the affinity moiety and target molecule, may be a receptor-ligand binding pair, specifically, where the affinity moiety is or comprises a receptor where the target molecule is the ligand or vice versa. More specifically, a receptor-ligand affinity pair refers to the specific and often high-affinity interaction between a receptor (a protein molecule usually located on the cell surface or within a cell) and a ligand (a molecule that binds to the receptor). Specificity of this binding as used herein is meant that each receptor typically binds to a particular ligand or a group of structurally similar ligands, thereby forming the receptor-ligand complex, that is detected and quantified by the disclosed bio transistor systems and methods, and often triggers a conformational change in the receptor, initiating a cascade of intracellular events. Example that may be useful in the present disclosure may include hormone receptors, neurotransmitter receptors, and the like. “Receptors” are chemical structures, composed of protein, that receive and transduce signals that may be integrated into biological systems. These signals are typically chemical messengers which bind to a receptor and produce physiological responses such as change in the electrical activity of a cell. Receptor proteins can be classified by their location. Cell surface receptors also known as transmembrane receptors, include ligand-gated ion channels, G protein-coupled receptors, and enzyme-linked hormone receptors. Intracellular receptors are those found inside the cell and include cytoplasmic receptors and nuclear receptors. A molecule that binds to a receptor is called a “ligand” and can be a protein, peptide, or another small molecule. A non-limiting embodiment for a receptor-ligand pair is provided by the present disclosure that use the receptor molecule HFIP as the affinity moiety in the disclosed bio transistor systems, that specifically recognizes and binds the small molecule target organophosphate molecule DCNP.

As noted above, an affinity pair composed of a small molecule and an amino-acid based molecule may be an Antibody-antigen pair. An “antibody (Ab)”, also known as an “immunoglobulin (Ig)”, is a large, Y-shaped glycoprotein used by the immune system to identify and neutralize foreign objects such as pathogenic bacteria and viruses. The antibody recognizes a unique molecule called an “antigen”. Each tip of the “Y” of an antibody contains a paratope (analogous to a lock) that is specific for one particular epitope (analogous to a key) on an antigen, allowing these two structures to bind together with precision. The term “antibody” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen or antigen-binding scaffolds. The antigen binding domains in accordance with the invention may recognize and bind a specific antigen or epitope. It should be therefore noted that the term “binding specificity”, “specifically binds to an antigen”, “specifically immuno-reactive with”, “specifically directed against” or “specifically recognizes”, when referring to an antigen or particular epitope, refers to a binding reaction which is determinative of the presence of the epitope in a heterogeneous population of proteins and other biologics.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics, as in the estriol molecule used according to some embodiments as the target molecule. Still further, as indicated above, an “antigen-binding domain” can comprise or consist of an antibody or antigen-binding fragment of an antibody.

Still further, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In yet some further embodiments, an affinity pair applicable for the present disclosure is an Enzyme-substrate pair.

Enzymes differ from most other catalysts by being much more specific. An enzyme's specificity comes from its unique three-dimensional structure. Enzymes are usually much larger than their substrates. Sizes range from just 62 amino acid residues, for the monomer of 4-oxalocrotonate tautomerase, to over 2,500 residues in the animal fatty acid synthase. Only a small portion of their structure (around 2-4 amino acids) is directly involved in catalysis: the catalytic site. This catalytic site is located next to one or more binding sites where residues orient the substrates. The catalytic site and binding site together compose the enzyme's active site. The remaining majority of the enzyme structure serves to maintain the precise orientation and dynamics of the active site. In some enzymes, no amino acids are directly involved in catalysis; instead, the enzyme contains sites to bind and orient catalytic cofactors. Enzyme structures may also contain allosteric sites where the binding of a small molecule causes a conformational change that increases or decreases activity. Enzymes must bind their substrates before they can catalyze any chemical reaction. Specificity is achieved by binding pockets with complementary shape, charge and hydrophilic/hydrophobic characteristics to the substrates. Enzymes can therefore distinguish between very similar substrate molecules to be chemoselective, regioselective and stereospecific. The enzyme component of the enzyme-substrate affinity pair can be a whole enzyme, and/or any fragments thereof, such as the enzyme's active site or the enzyme's binding site as long as it's binding capability is maintained.

It should be understood that although specifically directed at affinity moiety that comprises at least one small molecule and a proteineous molecule, the present disclosure further encompasses the use of any of the disclosed affinity pairs. more specifically, in some embodiments, the affinity pair may a nucleic acid sequence and a proteineous factor.

In some embodiments, the affinity pair may be aptamer-target affinity pair. “Aptamers” are short sequences of artificial DNA, RNA, XNA, or peptide that bind a specific target molecule, or family of target molecules. They exhibit a range of affinities (KD in the pM to μM range), and are sometimes classified as “chemical antibodies” or “antibody mimics”. The nucleic acid-based structure of aptamers, which are mostly oligonucleotides, is very different from the amino acid-based structure of antibodies, which are proteins. Aptamers are usually obtained by selection from a large random sequence library, using methods well known in the art, such as SELEX and/or Molinex. In SELEX, the best aptamers from a starting DNA library made of about a quadrillion different randomly generated pieces of DNA or RNA are repeatedly selects. After SELEX, the chemistry of the aptamers might be mutated or changed and another selection can be done, or a rational design processes might be used to engineer improvements.

In yet some further alternative embodiments, an affinity pair, for the affinity moiety and target molecule, may be a proteineous factor-specific nucleic acid binding site binding-pair. More specifically, a proteineous factor-specific nucleic acid binding site refers to a specific sequence or structure within a nucleic acid molecule (DNA or RNA) that is recognized and bound by a particular protein factor, that may be a protein, often a regulatory protein such as a transcription factor, RNA-binding protein, or enzyme, that interacts with nucleic acids. Non-limiting examples for such proteins, are proteins involved in various cellular processes, including gene expression, replication, and RNA processing. The nucleic acid binding site, that may be used in the present disclosure as the affinity moiety, is a specific region on a nucleic acid (DNA or RNA) where the protein binds. The binding site is typically a specific sequence of nucleotides or a particular structural motif that the protein recognizes and interacts with.

In yet some further alternative embodiments, an affinity pair, for the affinity moiety and target molecule, may be an aptamer-aptamer target binding pair. More specifically, an aptamer-aptamer target binding pair, as used herein, refers to the specific and high-affinity interaction between an aptamer and its corresponding target molecule. Aptamers according to some embodiments, are short, single-stranded DNA or RNA molecules that can fold into unique three-dimensional structures, allowing them to bind selectively to a wide range of targets, including proteins, small molecules, ions, and even whole cells. Thus, in some embodiments, the affinity moiety of the disclosed bio transistor systems is, composed of, or comprising, at least one aptamer, for example, a nucleic acid-based aptamer. Accordingly, the target molecule recognized by such affinity moiety may be a protein, small molecule, ion, and even a whole cell. It should be further noted that aptamer-target binding is mediated by non-covalent interactions, including hydrogen bonds, electrostatic interactions, Van der Waals forces, and sometimes hydrophobic interactions.

In some embodiments, the affinity moiety and the specific target molecule may form an affinity pair composed of an enzyme-substrate. More specifically, “affinity pair” highlights the complementary nature of the enzyme and substrate, where the enzyme's active site is specifically shaped to recognize and bind its substrate, resulting in a high-affinity interaction and high specificity. This interaction is central to the enzyme's catalytic function and involves specificity, that is meant herein that each enzyme binds to a particular substrate or a group of closely related substrates, affinity, that relates to the strength of the binding between an enzyme and its substrate is referred to as affinity. High-affinity binding ensures that the substrate is efficiently captured by the enzyme. This binding is often mediated by non-covalent interactions such as hydrogen bonds, ionic interactions, and Van der Waals forces. Thus, in some embodiments, the formation of the enzyme-substrate complex, is referred to herein as binding of the target molecule to the affinity moiety that is detected and/or quantified by the bio transistor systems and methods of the present disclosure. Formation of the enzyme-substrate complex is followed by catalysis and release of the products from the enzyme's active site.

As indicated above, in some embodiments, the at least one affinity pair is receptor-ligand affinity pair. In some embodiments, the at least one affinity moiety and the at least one target molecule are derived from a receptor-ligand binding pair. In some embodiments, the affinity moiety is derived from a receptor molecule. Accordingly, the target is derived from a ligand for the receptor molecule. In yet some alternative embodiments, the affinity moiety is derived from a ligand molecule. Accordingly, the target is derived from a receptor for the ligand molecule.

The target recognition moiety, that is also referred to herein as an affinity moiety, comprise in some embodiments an amino acid-based molecule (e.g., receptor or an antibody) that specifically recognizes and binds the target small molecule. “Specifically binds” as used herein refers to an affinity moiety which interacts with a specific target molecule, while avoiding interactions with other molecules. Examples of moieties with specific binding capabilities include for example, receptors, antibodies, nucleic acid binding sites, aptamers, as well as enzymes. The target recognition component, also referred to herein as the affinity moiety, specifically, the antibody or the receptor, specifically binds the target molecule in any stoichiometric ratio. In some embodiments, one target recognition component binds at least one, at least two, at least three, at least four, at least five, at least ten, at least hundred, at least thousand and even more, target molecules. In some embodiments, the target-recognition component, specifically, binding site, comprises more than one moiety, wherein the plurality of moieties may be the same and/or may be different (e.g. different binding sites, etc).

In some particular embodiments, the target molecule is at least one ligand comprising a small molecule chemical substance, and the affinity moiety comprises an amino acid-based receptor molecule for the ligand.

In some additional embodiments, the chemical substance is toxic for at least one eukaryotic organism.

In some embodiments the eukaryotic organism is at least one organism of the biological kingdom Animalia or of the biological kingdom Plantae. For Example, the eukaryotic organism of the biological kingdom Animalia may be an insect, avian, fish and a mammalian subject.

In some further embodiments, the chemical substance is organophosphate.

In some further embodiments, the organophosphate comprises at least one of pesticides, herbicides, and/or chemical warfare agents.

In some specific embodiments, the at least one organophosphate is diethylcyanophosphonate (DCNP). Diethylcyanophosphonate (DCNP), as used herein, is an organophosphorus compound with the chemical formula (C₂H₅O)₂P(O)CN. It is used in organic synthesis, particularly in the formation of phosphonate esters. DCNP consists of a phosphonate group (P(O)(OR)₂) attached to a cyano group (CN). The two ethoxy groups (C₂H₅O) are bonded to the phosphorus atom, having a molecular weight of 151.11 g/mol. In some embodiments, the receptor is hexafluoroisopropanol (HFIP). Accordingly, in some embodiments, the affinity moiety of the disclosed bio-transistor systems, may comprise HFIP or any functional fragment thereof. More specifically, Hexafluoroisopropanol (HFIP) is a fluorinated alcohol with the chemical formula (CF₃)₂CHOH. It is widely used in organic chemistry and materials science due to its unique properties. HFIP consists of a central isopropanol structure where all six hydrogen atoms on the carbon backbone are replaced with fluorine atoms, giving it the chemical formula (CF₃)₂CHOH, having a molecular weight of 168.04 g/mol. Hexafluoroisopropanol (HFIP) can act as a receptor for diethylcyanophosphonate (DCNP) through non-covalent interactions, primarily hydrogen bonding and possibly dipole interactions. HFIP has a hydroxyl group that can form strong hydrogen bonds with the electronegative atoms in DCNP, such as the oxygen atoms in the ethoxy groups and the nitrogen atom in the cyano group. This hydrogen bonding can facilitate the formation of a stable complex between HFIP and DCNP.

In some embodiments of the present disclosure, the at least one sample is a biological sample and/or an environmental sample. A “sample” as used herein may be any biological and/or environmental sample. More specifically, an “Environmental sample” refers to any sample derived from any media or material in the environment. The most common environmental samples are air, water, soil, biological materials, and wastes (liquids, solids or sludges) such as sewage. Environmental sampling is typically performed to determine the presence of hazardous materials in any media or material, including indoor or outdoor air, soil, groundwater, surface water or building materials. Still further, in some embodiments, a sample may comprise any food product or any byproduct derived from any industrial activity or facility, or any water reservoir that may be contaminated by the disclosed organophosphates. In yet some further alternative embodiments, the sample may be a “biological sample”, specifically, a biological material collected from living and/or deceased organisms and/or living and/or dried out plants or their environment. There are many different types of biological samples, including biofluids, tissue, cells and other. Biological samples can be obtained from the body via several different methods such as excretion (e.g. urine), secretion (e.g. breast milk) or extraction (e.g. blood). Non limiting examples of biological samples include blood, bile, bone marrow aspirate, breast milk/mammary gland milk, Cerebral Spinal Fluid (CSF), feces, plasma, saliva, semen, serum, sputum, sweat, as well as oral, nasal and vaginal fluids (typically collected using a swab), and synovial fluid, tears and urine. Biological samples include also cells such as epithelial cells, fibroblasts, immune cells (e.g. T cell, B cells, NK cells etc.), peripheral blood mononuclear cells (PBMCs), red blood cells (RBCs), buffy coat, bone marrow mononuclear cells, dissociated tumor cells, mesenchymal stem cells, myoblasts, hepatocytes, etc as well as tissues. Cell samples are collected and isolated from either tissue samples, biofluid samples, or biopsy samples. Depending on the cell type, specific cell isolation protocols are required in order to obtain the purified cell sample.

Biological material collected from plants may be for example any sample derived from the leaf, roots, stems, nectar, seeds and/or fruits of any plant.

In some further embodiments, the sample is an environmental sample, particularly where the target molecule is an organophosphate molecule, for example, DCNP.

In some embodiments, the at least one affinity pair of the bio-transistor system of the present disclosure is an antigen-antibody affinity pair, where the antigen in this pair is a small molecule target antigen recognized by the amino-acid based affinity moiety that is composed of an antibody specific for the small molecule target.

In yet some further embodiments, the target is at least one antigen comprising a small molecule steroid compound. Accordingly, the affinity moiety of the disclosed bio-transistor system comprises an antibody specific for the antigen, specifically, the small molecule steroid compound. Thus, in yet some further embodiments of the disclosed bio-transistor system, the target molecule is, or comprises a small molecule steroid compound. In some embodiments, the target small molecule steroid compound is a molecule naturally produced by at least one eukaryotic organism.

In yet some further embodiments of the disclosed bio-transistor system, the target small molecule steroid compound is of the estrogen family. In yet some further specific embodiments, the small molecule steroid compound is Estriol.

Estriol (uE3) as used herein, is a naturally occurring estrogen and one of the three main estrogens produced by the human body, specifically, the fetus and placenta, alongside estradiol and estrone, having the chemical formula C₁₈H₂₄O₃, a molecular weight of 288.38 g/mol, and IUPAC Name (8R,9S,13S,14S,16R,17S)-13-Methyl-6,7,8,9,11,12,14,15,16,17-decahydrocyclopenta[a]phenanthrene-3,16,17-triol. Formula I disclose the chemical structure of estriol. Estriol has three hydroxyl groups located at positions 3, 16, and 17 on the steroid backbone, making it a triol. Estriol is synthesized primarily in the placenta during pregnancy. It is a metabolite of estrone and estradiol, both of which are precursors in the biosynthetic pathway. Estriol is the dominant estrogen during pregnancy. Its levels increase significantly as pregnancy progresses and are considered an indicator of fetal well-being and placental function. Low levels can be associated with chromosomal abnormalities.

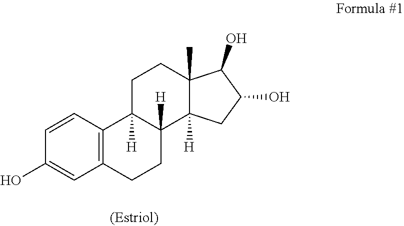

In some embodiments, the affinity moiety of the bio-transistor system disclosed herein is an antibody molecule. As the target is estriol, the affinity moiety is or comprises at least one anti-Estriol antibody or any functional fragments thereof.

In yet some further embodiments of the disclosed bio-transistor system, the at least one sample is a biological sample and/or environmental sample. Specifically, any of the biological samples disclosed above. Accordingly, the disclosed bio-transistor system is configured for determining the presence and/or the quantity of the estriol in a sample, specifically, a biological sample.

In some specific and non-limiting embodiments, such sample may be a blood and/or serum sample. In yet some further embodiments, the sample is a serum sample obtained from a pregnant female subject.

As indicated herein, in some embodiments, the disclosed bio transistor system may be useful in screening for compounds that may modulate the interaction between the two components of the binding pair, specifically, any molecule that decreases or alternatively decreases the interaction between the affinity moiety and the target molecule. This aspect will be elaborated in the screening method described herein after. Accordingly, the sample may further comprise a candidate compound that is evaluated for its ability to change the interaction (e.g., binding) of the affinity moiety (e.g., a receptor and/or n antibody), and its specific target (e.g., a ligand or a small molecule antigen). Such candidate molecule may be any molecule. Thus, in some embodiments of the present disclosure, the at least one sample further comprises at least one candidate compound that modulates the interaction between the affinity moiety and the target molecule. The candidate compound comprises at least one of: an amino acid-based molecule, a nucleic acid-based molecule, a small molecule, a carbohydrate-based molecule, a lipid-based molecule, or any combination thereof. In some embodiments of the present disclosure, the disclosed screening method is particularly applicable for screening of a compound that inhibits the interaction of the affinity moiety with the target molecule. More specifically, a compound that “inhibits the interaction” as used herein, refers to a compound that reduces, prevents, blocks, impedes, suppresses, prevents, hinders and/or restricts interaction between the at least one affinity moiety and the target molecule either partially, or completely. For example, any inhibition of between about 10-50%, 51-80%, 81-99% or even, of 100% of the interaction or binding between the affinity moiety and the target molecule.

Another aspect of the present disclosure relates to a battery comprising two or more of the bio-transistor systems as defined above.

In some embodiments, the battery of the present disclosure comprises two or more bio-transistor systems. In some embodiments, one of these at least two bio-transistor systems comprise the estriol-anti-estriol antibody as defined above. In yet some further embodiments, the at least one additional bio-transistor system of the disclosed battery may comprise at least one affinity moiety for at least one additional maternal serum marker. A maternal serum marker, as used herein, is a substance found in a pregnant woman's blood that can provide important information about the health and development of the fetus, as well as the mother's health during pregnancy. These markers are typically proteins, hormones, or other molecules that are produced by the placenta, the fetus, or the mother's body in response to pregnancy. Maternal serum markers are used in prenatal screening tests to assess the risk of certain fetal conditions, such as chromosomal abnormalities (e.g., Down syndrome), neural tube defects, and other genetic or developmental disorders. They can also help monitor the health of the pregnancy and the risk of complications such as preeclampsia. Common maternal serum markers encompassed by the present disclosure include, but are not limited to AFP, hCG, Estriol (uE3), Inhibin A, PAPP-A (Pregnancy-Associated Plasma Protein A), and the like.

Accordingly, in some embodiments, the present disclosure provides a battery of bio transistor systems comprising:
(a), at least one bio-transistor system comprising at least one affinity moiety specific for Estriol. In some specific embodiments, the affinity moiety is or comprises at least one anti-Estriol antibody. The disclosed battery further comprises at least one of:
(b), at least one bio-transistor system comprising at least one affinity moiety specific for alpha-fetoprotein (AFP). In some embodiments, the affinity moiety is or comprises at least one anti-AFP antibody. The disclosed battery comprises additionally, or alternatively, (c), at least one bio-transistor system comprising at least one affinity moiety specific for human chorionic gonadotropin (hCG). In some embodiments, the affinity moiety is, or comprises at least one anti-hCG antibody.

More specifically, Alpha-fetoprotein (AFP) is a glycoprotein produced primarily by the fetal liver, yolk sac, and gastrointestinal tract. It is a major plasma protein in the developing fetus and plays a role similar to that of serum albumin in adults. AFP is also produced in certain pathological conditions in adults, making it a valuable biomarker in clinical diagnostics. AFP is a single-chain glycoprotein composed of approximately 590 amino acids. The protein has carbohydrate moieties attached to specific asparagine residues, contributing to its stability and solubility. AFP contains three homologous domains, each contributing to its overall function and binding properties. These domains are involved in the transport and binding of various ligands, such as fatty acids and bilirubin. AFP binds and transports various molecules, including fatty acids, bilirubin, and steroids, and is thus essential for fetal development.

In some embodiments, AFP, as used herein refers to the human AFP, that comprises the amino acid sequence as denoted by UniProt accession number P02771. In yet some further embodiments, the human AFP as used herein comprises the amino acid sequence as denoted by SEQ ID NO: 1, or any homologs or variants thereof.

In yet some further embodiments, Human Chorionic Gonadotropin (hCG), as used herein is a hormone produced by the placenta. Abnormal levels can indicate issues like multiple pregnancies or risk of chromosomal abnormalities. In some embodiments, hCG, as used herein refers to the human hCG, that comprises the amino acid sequence as denoted by UniProt accession number P01215. In yet some further embodiments, the human hCG as used herein comprises the amino acid sequence as denoted by SEQ ID NO: 2, or any homologs or variants thereof.

Accordingly, the present disclosure provides in some aspects thereof, a battery of bio transistors useful for monitoring pregnancy and female health.

Another aspect of the present disclosure related to a method for determining presence and/or quantity of at least one target molecule in at least one sample. The method comprising: in step (a), contacting the at least one sample with a bio-transistor having an active region carrying at least one affinity moieties, or a battery comprising at least two of the bio-transistors. It should be understood that each affinity moiety is specific for a target molecule. It should be noted that in some embodiments, the one or more affinity moieties are selected moieties. In yet some further embodiments, the bio transistor or battery used by the disclosed methods may comprise one or more different or similar or identical affinity moieties. It should be further noted that each one of the at least one affinity moiety is specific for one target molecule. In step (b), performing one or more measurements, each measurement comprising: applying a selected electric potential on the sample, and determining current transmission profile through a channel of the bio-transistor with respect to potential variation of at least one gate electrode of the bio-transistor; and (c) processing data on the current transmission through the channel for one or more selected gate potential and one or more selected electric potential values applied on the sample and determining presence and/or quantity of the one or more or the at least one target molecules in accordance with pre-stored calibration data.

In some embodiments of the present disclosure, the bio-transistor used by the method comprising: (i) at least one channel; (ii) source and drain electrodes; (iii) at least one gate electrode; and (iv) at least one additional electrode positioned to be in electrical contact with the sample. The at least one active region is located in proximity to the channel region, separated from the channel region by an electrically insulating layer. The control unit thereby configured to determine data on presence and/or quantity of one or more target molecules in the sample.

In some further embodiments, the method comprising performing two or more measurements, wherein each measurement comprises applying respective selected different potential on the sample.

In some other embodiments, the bio-transistor used in the disclosed methods is as defined by the present disclosure, herein above.

In some embodiments, the methods and systems of the present disclosure may be applicable for any organism of the biological kingdom Animalia. In more specific embodiments, such organism may be any unicellular or multicellular invertebrate or vertebrate organism. More specifically, invertebrates, may be organisms of the Phylum Porifera-Sponges, the Phylum Cnidaria-Jellyfish, hydras, sea anemones, corals, the Phylum Ctenophora-Comb jellies, the Phylum Platyhelminthes-Flatworms, the Phylum Mollusca-Molluscs, the Phylum Arthropoda-Arthropods, the Phylum Annelida-Segmented worms like earthworm and the Phylum Echinodermata-Echinoderms. Still further, in some embodiments, the methods of the present disclosure may be applicable for any vertebrate organism, specifically, any organism derived from any of the vertebrates groups that include Fish, Amphibians, Reptiles, Birds and Mammals (e.g., Marsupials, Primates, Rodents and Cetaceans). In some particular embodiments, the methods of the present disclosure may be applicable for a mammal (specifically, at least one of a human, Cattle, rodent, domestic pig (swine, hog), sheep, horse, goat, alpaca, lama and Camels).

More specifically, in some embodiments, as indicated herein, the methods of the present disclosure may be applicable for a vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans).

Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish.

Still further, in some embodiments, the disclosed bio transistor systems and methods may be applicable for samples obtained from any one of a human or non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the subject of the present disclosure may be a mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the present disclosure may be applicable for any organism of the order primates. More specifically, primates are divided into two distinct suborders, the first is the strepsirrhines that includes lemurs, galagos, and lorisids. The second is haplorhines—that includes tarsier, monkey, and ape clades, the last of these including humans. In yet some further embodiments, the present disclosure may be applicable for any organism of the subfamily Homininae, that includes the hylobatidae (gibbons) and the hominidae that includes ponqunae (orangutans) and homininae [gorillini (gorilla) and hominini ((panina (chimpanzees) and hominina (humans))]. Thus, in some embodiments, a subject as disclosed herein relates to a human subject. In some embodiments, the human subject may be of any sex, ethnic group, age or physical or mental condition.

In some specific embodiment, the methods of the present disclosure may be applicable for a mammal that may be at least one of a Cattle, domestic pig (swine, hog), sheep, horse, goat, alpaca, lama and Camels.

More specifically, the subject the present disclosure as well as the methods disclosed herein above offer great economic advantage for any industrial or agricultural use of animals, specifically, livestock. Thus, in some specific embodiments, the present disclosure may be applicable for mammalian livestock, specifically those used for meat, milk and leather industries. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the subject of the present disclosure may be Cattle, colloquially cows, that are the most common type of large, domesticated ungulates, that belong to the Bovidae family.

In yet some further embodiments, the organism applicable in the methods of the present disclosure, may be avian organisms. In yet some further specific embodiments, the present disclosure may be suitable for birds. More specifically, domesticated and undomesticated birds are also suitable organisms for the present disclosure.

Still further, in some embodiments the disclosed bio transistor systems and methods may be applicable for samples obtained from organism of the biological kingdom Plantae that may be a dioecious plant, specifically, a plant presenting biparental reproduction. In some specific embodiments, the plant may be of the family Cannabaceae, specifically, any one of Cannabis (hemp, marijuana) and Humulus (hops). In more specific embodiments, the plant of the family Cannabaceae may be Cannabis (hemp, marijuana). In yet some further embodiments, the plant of the family Cannabaceae may be Humulus (hops).

In some embodiments, any plants are applicable in the present disclosure, for example, any model plants such as, Arabidopsis, Tobacco, Solanum licopersicum, Solanum tuberosum. In yet some further embodiments, Canola, Cereals (Corn wheat, Barley), rice, sugarcane, Beet, Cotton, Banana, Cassava, sweet potato, lentils, chickpea, peas, Soy, nuts, peanuts, Lemna, Apple, may be applicable in the present disclosure.

A non-comprehensive list of useful annual and perennial, domesticated or wild, monocotyledonous or dicotyledonous land plant or Algae—(i.e unicellular or multicellular algae including diatoms, microalgae, ulva, nori, gracilaria), applicable in accordance with the present disclosure may include but are not limited to crops, ornamentals, herbs (i.e., labiacea such as sage, basil and mint, or lemon grass, chives), grasses (i.e., lawn and biofuel grasses and animal feed grasses), cereals (i.e., rice, wheat, rye, oats, corn), legumes (i.e. soy, beans, lentils, chick peas, peas, peanuts), leafy vegetables (i.e. kale, bok-choi, cress, lettuce, spinach, cabbage), Amaranthacea (i.e. sugar beet, beet, quinoa, spinach), Compositea (i.e. sunflower, lettuce, aster), Malvaceae (i.e. cotton, cacao, okra, hibiscus), cucurbits (i.e., cucumber, squash, melon, watermelon), Solanaceous species (i.e tobacco, potato, tomato, petunia and pepper), Umbellifera (i.e. carrot, celery, dill, parsley, cumin), Crucifera (i.e., oilseed rape, mustard, brassicas, cauliflower, radish), Sesame, the monocot Aspargales (i.e. onion, garlic, leek, asparagus, vanilla, lilies, tulips, narcissus), Myrtacea (i.e., Eucalyptus, pomegranate, guava), Subtropical fruit trees (i.e. Avocado, Mango, Litchi, papaya), Citrus (i.e. orange, lemon, grapefruit), Rosacea (i.e. apple, cherry, plum, almond, roses), berry-plants (i.e. grapes, mulberries, blueberries, raspberry, strawberry), nut trees (i.e. macademia, hazelnut, pecan, walnut, chestnuts, brazil nut, cashew), banana and plantain, palms (i.e., oil-palm, coconut and dates), evergreen, coniferous or deciduous trees, woody species.

In some embodiments, the disclosed methods are applicable for eukaryotic organism that may be at least one organism of the biological kingdom Animalia or of the biological kingdom Plantae. For example, the eukaryotic organism of the biological kingdom Animalia may be an insect, avian, fish and a mammalian subject.

In some particular embodiments, the bio transistor system used by the disclosed method is based on a receptor-ligand binding pair that comprise at least one ligand comprising a small molecule chemical substance, and at least one affinity moiety comprising an amino acid-based receptor molecule for the ligand.

In some additional embodiments, the chemical substance used herein as the target molecule, is a small molecule toxic for at least one eukaryotic organism.

In some further embodiments, the toxic small molecule chemical substance is at least one organophosphate.

In other embodiments, the comprise at least one of pesticides, herbicides, and/or chemical warfare agents.

In some specific embodiments, the at least one organophosphate is diethylcyanophosphonate (DCNP).

In further embodiments, the receptor is hexafluoroisopropanol (HFIP).

In some embodiments of the present disclosure, the at least one sample is a biological sample and/or environmental sample.

Thus, in some embodiments, the present disclosure provides methods particularly useful for detecting, quantifying and/or monitoring organophosphate contamination in a sample, specifically, an environmental sample.

In some embodiments, the disclosed bio transistor systems and methods provide a sensitive and specific tool for detecting organophosphates in a sample, specifically a an environmental sample. In some embodiments, the disclosed bio transistor systems and methods detect organophosphate such as DCNP in a sample in an amount of about 1 pg/ml to about 100 ug/ml.

In some further embodiments, the present disclosure provides methods for determining the presence and/or the quantity of a target molecule produced naturally in an organism. For example, any of the eukaryotic organisms disclosed by the present disclosure, specifically, at least one mammalian organism. In more specific embodiments, the disclosed methods are particularly applicable for determining the presence and/or the quantity of a target molecule that is at least one antigen comprising a small molecule steroid compound. Accordingly, the at least one affinity moiety of the bio-transistor systems and/or at least one battery thereof used by the disclosed methods, may comprise an antibody specific for such antigen. Specifically, at least one antibody specific for the target small molecule steroid compound.

In some embodiments, the small molecule steroid compound used as a target by the disclosed methods is a small molecule steroid compound naturally produced by at least one eukaryotic organism. Specifically, at least one small molecule steroid compound produced by a human subject.

In some specific embodiments of the disclosed methods, the small molecule steroid compound is of the estrogen family. In some specific embodiments, the small molecule steroid compound is Estriol. Accordingly, the at last one affinity moiety of the bio-transistor system used in the disclosed methods is at least one antibody, specifically, at least one anti-Estriol antibody or any functional fragments thereof.

In yet some further embodiments, the disclosed methods are applicable for determining the presence and/or quantity of at least one small molecule steroid compound, specifically of estriol in a sample, specifically a biological sample. In some specific embodiments the biological sample may be a blood sample and/or a serum sample. In yet some further embodiments, the sample is a serum sample obtained from a pregnant female subject.

In some embodiments, the disclosed bio transistor systems and methods provide a sensitive and specific tool for detecting Estriol in a sample, specifically a biological sample such as serum sample. In some embodiments, the disclosed bio transistor systems and methods detect Estriol in a serum sample in an amount of about 1 fg/ml to about 10 μg/ml.

In some further embodiments, particularly in case the disclosed methods use any of the batteries disclosed herein, the present disclosure provides methods for determining the presence and/or quantity of at least one maternal serum marker. The maternal serum marker comprises estriol, as well as at least one additional maternal serum marker. Thus, in some particular embodiments, the present disclosure provides methods using a battery of bio transistor systems comprising:
(a), at least one bio-transistor system comprising at least one affinity moiety specific for Estriol. In some specific embodiments, the affinity moiety is or comprises at least one anti-Estriol antibody. The disclosed battery further comprises at least one of:
(b), at least one bio-transistor system comprising at least one affinity moiety specific for alpha-fetoprotein (AFP). In some embodiments, the affinity moiety is or comprises at least one anti-AFP antibody. The disclosed battery comprises additionally, or alternatively, (c), at least one bio-transistor system comprising at least one affinity moiety specific for human chorionic gonadotropin (hCG). In some embodiments, the affinity moiety is, or comprises at least one anti-hCG antibody.

The present disclosure thus provides effective methods for determining and monitoring pregnant female subjects during pregnancy.

Another aspect of the current disclosure is a diagnostic method for determining a physiological and/or environmental condition or state of a subject and/or a media and/or a habitat. The method comprising: (a) contacting the at least one sample with a bio-transistor having an active region at least one affinity moiety, or a battery comprising at least two of said bio-transistors. In some embodiments, each affinity moiety is specific for a target molecule. In yet some further embodiments, the active region is modified to include the disclosed affinity moieties. In step (b), performing one or more measurements, each measurement comprising: applying a selected electric potential on the sample, and determining current transmission profile through a channel of the bio-transistor with respect to potential variation of at least one gate electrode of the bio-transistor. Step (c) involves processing data on the current transmission through the channel for one or more selected gate potential and one or more selected electric potential values applied on the sample. In step (d), determining presence and/or quantity of the one or more target molecules in accordance with pre-stored calibration data, thereby obtaining a target molecule value for the sample. In step (e), determining that the subject and/or media and/or habitat display the physiological and/or environmental condition or state, if the at least one target molecule value obtained for the sample in step (d), is positive or negative with respect to a reference target molecule value pre-determined for the physiological and/or environmental condition or state, or with respect to a target molecule value determined for at least one control sample.

In some embodiments, the bio-transistor system used by the diagnostic methods is as defined by the present disclosure herein above.

In some embodiments, the physiological state and/or condition of a subject comprises pathological condition/s and/or health condition/s in the subject.

In some embodiments, the physiological state and/or condition includes the nerve system, the respiratory system, the skin, the metabolic state, behavioral and/or mental state and/or organ failure, tissue damage, etc.

In some embodiments, the bio-transistor system and/or battery used by the disclosed diagnostic methods, may comprise at least one affinity moiety comprising HFIP. More specifically, the affinity moiety of the bio-transistor system used by the methods is specific for the target DCNP. It should be understood that the at least one affinity moiety used by the disclosed methods is or comprises HFIP that is a receptor molecule for the organophosphate DCNP. In yet some further embodiments, the method is for the diagnosis of organophosphate/s contamination in a media and/or habitat.

In some embodiments, the disclosed methods are applicable for the diagnosis of a pathological condition in a subject, caused by, or associated with exposure to a media and/or habitat contaminated by the organophosphate/s. specifically, any pathologic disorder caused by exposure to DCNP.

More specifically, an organophosphate is a chemical compound that contains a phosphate group bonded to an organic moiety. Organophosphates are widely used in various industries, including agriculture, medicine, and chemical manufacturing. Organophosphates contain the general structure (RO)₃P═O or (RO)₂P(O)R′, where “R” represents an organic group (alkyl or aryl), and “P” represents a phosphorus atom double bonded to an oxygen atom (phosphate group). They can also include other variations, such as thiophosphates (where sulfur replaces oxygen) and phosphonates. Organophosphates are commonly used as insecticides. Examples include malathion, parathion, and chlorpyrifos. They act by inhibiting acetylcholinesterase, an enzyme essential for nerve function in insects, leading to their death. Some organophosphates, such as sarin, tabun, and VX, are highly toxic and used as chemical warfare agents. They also inhibit acetylcholinesterase, causing severe neurological effects in humans. Organophosphates are employed as plasticizers, flame retardants, and lubricants in various processes. Organophosphates inhibit acetylcholinesterase, an enzyme that breaks down acetylcholine, a neurotransmitter. This inhibition leads to an accumulation of acetylcholine in synapses, causing continuous stimulation of muscles, glands, and central nervous system pathways. It should be appreciated that pathologic disorders caused by exposure to organophosphates, include acute toxicity, as well as chronic exposure. More specifically, symptoms of acute organophosphate poisoning include headache, dizziness, nausea, vomiting, abdominal pain, muscle weakness, and respiratory distress. Severe cases can lead to convulsions, coma, and death. Still further, chronic exposure include long-term exposure to low levels of organophosphates can result in neurological and cognitive impairments, including memory loss, mood changes, and peripheral neuropathy.

In yet some further alternative embodiments, the present disclosure provides diagnostic methods based on detection and determination of the quantity of estriol. Accordingly, the disclosed diagnostic methods use the bio-transistor systems and/or battery that comprise at least one affinity moiety specific for estriol. In some embodiments, such affinity moiety is or comprises at least one anti-estriol antibody. In yet some further embodiments, step (d) of the disclosed diagnostic methods results in obtaining the estriol value for a maternal serum sample. Thus, in some embodiments, the detection and/or quantification of the estriol levels by the disclosed bio-transistor system, is of a diagnostic value. More specifically, in some embodiments, a level of the target estriol determined by the disclosed bio-transistor systems, that is below a standard level (also referred to herein as “negative”), is indicative of at least one genetic disorder associated with chromosomal abnormalities. In some specific embodiments, such chromosomal abnormalities comprise at least one trisomy. In more specific embodiments, low levels of estriol, as compared to a standard level, is indicative of a trisomy 21, for example, Down Syndrome (DS), and/or a trisomy 18, for example, Edward's Syndrome. Standard estriol levels for non-pregnant adults are 80-130 fg/mL, for pregnant women levels can be much higher and vary depending on the stage of pregnancy. The highest sensitivity of 77.56 I_{Normalized} per dec, for a dynamic range of 11 orders of magnitude (1 fg/ml to 10 μg/ml) for estriol with LOD of 1 fg/ml is achieved. Estriol secretion is increased by 1000 times during pregnancy due to placental synthesis reaching at 37 weeks onwards a concentration of 80-130 pg/ml.

In some further embodiments, the present disclosure provides diagnostic methods that display a profile of maternal serum markers. More specifically, in some embodiments the diagnostic methods of the present disclosure may use a battery of two or more of the bio-transistor systems disclosed herein. In some specific embodiments, the two or more of the bio-transistor systems of the battery used by the disclosed diagnostic methods may comprise, in addition to a bio-transistor system comprising estriol specific antibody, also at least one bio-transistor system comprising at least one affinity moiety for at least one additional maternal serum marker. According to such embodiments, the battery used by the disclosed diagnostic methods may comprise:
(i), at least one bio-transistor system comprising at least one affinity moiety specific for Estriol. In some embodiments, the affinity moiety comprises anti-Estriol antibody. The battery used by the disclosed diagnostic methods may further comprise at least one of: (ii), at least one bio-transistor system comprising at least one affinity moiety specific for alpha-fetoprotein (AFP). In some embodiments, the affinity moiety comprises anti-AFP antibody. The battery used by the disclosed diagnostic methods may comprise additionally, or alternatively, (iii), at least one bio-transistor system comprising at least one affinity moiety specific for human chorionic gonadotropin (hCG). In some embodiments, the affinity moiety may comprise anti-hCG antibody.

Thus, in some embodiments step (d) of the disclosed diagnostic methods results in obtaining the estriol value for at least one maternal serum sample, and optionally of at least one of, the AFP value and/or the hCG value of the at least one maternal serum sample.

In some specific and non-limiting embodiments, the bio-transistor system of the present disclosure is specifically designed for detecting and/or quantifying a target molecule that is the Alpha-fetoprotein (AFP). Accordingly, in some embodiments, the at least one affinity moiety carried by the active region of the bio-transistor system of the present disclosure comprises, is, or is derived from, at least one antibody specific for AFP.

Thus, in some embodiments, the detection and/or quantification of the estriol and at least one of AFP and hGC levels by the disclosed bio-transistor systems and battery, is of a diagnostic value. More specifically, in some embodiments, a level of the target estriol and at least one of AFP and hGC determined by the disclosed bio-transistor systems, that is below a standard level (also referred to herein as a “negative”), is indicative of at least one genetic disorder associated with chromosomal abnormalities. In some specific embodiments, such chromosomal abnormalities comprise at least one trisomy. In more specific embodiments, low levels of estriol, and of AFP and/or hGC, as compared to a standard level, is indicative of a trisomy 21, for example, Down Syndrome (DS), and/or a trisomy 18, for example, Edward's Syndrome. Standard AFP levels for non-pregnant adults are less than 10 ng/mL, for pregnant women levels can be much higher and vary depending on the stage of pregnancy. The highest sensitivity of 25.98 readout per dec, for a dynamic range of 1.05 pM-10.5 nM with LOD of 10.5 aM achieved. Standard Estriol levels for non-pregnant adults are less than 150 fg/mL, for pregnant women levels can be much higher and vary depending on the stage of pregnancy. The highest sensitivity of 77.56 I_{Normalized} per dec, for a dynamic range of 1 fg/ml to 10 ug/ml with LOD of 1 fg/ml.

As indicated above, the disclosed methods involve in the first step determination of the level of specific target molecule to obtain the value for each target in the sample, as will be elaborated herein after. The next step involves determination if the expression value is positive or negative. It should be understood that determination of a “positive” or alternatively “negative” value of the target molecule levels and/or amount with respect to a standard value or a control value may involve in some embodiments comparison of the value determined for the quantity and/or mount) of the target molecule of the examined sample as obtained in step (d), with the amount/level value of the target obtained for a control sample, or from any established or predetermined value of the target molecule level and/or amount and/or quantity (e.g., a standard value) obtained from a known control (either healthy controls or of subjects suffering from a pathological disorder). Thus, in some embodiments, “positive” is meant a value that is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the amount/level/quantity value of the reference and/or the standard value of a healthy control, any other suitable control or any other predetermined standard. Still further, a “negative” value in some embodiments may be a reduced, low, non-existing or lack of expression of a target molecule in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the value of the amount of the target molecule in a healthy control, any other suitable control or any other predetermined standard.

As used herein, “healthy controls” or “healthy population” may refer to a population of subjects that does not suffer from a disease of interest or refer to a population before appearance of a disease of interest. In some embodiments, the value of the target molecule in a control population refers to a baseline level of the target molecule of a healthy population or to a baseline level of the target molecule before appearance of a disease in a studied population. In some other embodiment, a “healthy control” or “control” may refer to the to a baseline level of the target molecule before appearance of a disease in a specific subject.

In yet some further embodiments, “positive”, specifically, higher, elevated levels and/or amount and/or quantity when compared to a control, the subject is classified as a subject that display a specific disorder as indicated herein for each of the target molecules.

It should be appreciated that a “reference value” or a “Standard”, or a “predetermined standard” or a “predetermined reference value” as used herein, denotes either a single standard value or a plurality of standards with which the level of at least one of the target molecule/s from the tested sample is compared. The standards may be provided, for example, in the form of discrete numeric values or in the form of a comparative curve prepared on the basis of such standards (standard curve).

Thus, in yet more specific embodiments, the method of the invention involves comparing the values of the amount and/or quantity of the target molecule/s determined for the tested sample with predetermined standard values or cutoff values, or alternatively, with the values of at least one control sample. As used herein the term “comparing” denotes any examination of the level and/or amount and/or quantity values obtained in the samples disclosed herein as detailed throughout in order to discover similarities or differences between at least two different samples. It should be noted that in some embodiments, comparing according to the present disclosure encompasses the possibility to use a computer-based approach.

Step (a) of the disclosed methods involves the action of contacting the bio transistors of the present disclosure with the examined sample. The term “contacting” means to bring, put or incubates together. As such, a first component, e.g., affinity moiety is contacted with a second component, specifically, the target molecule when the two components are brought or put together, e.g., by touching them to each other or combining them. In the context of the present disclosure, the term “contacting” includes all measures or steps which allow interaction between the at least one of the affinity moieties of at least one of the target molecules. The contacting is performed in a manner so that the at least one of affinity moieties of at least one of the target molecules, can interact with or bind to the target molecule in the tested sample. The binding will preferably be non-covalent, reversible binding, e.g., binding via salt bridges, hydrogen bonds, hydrophobic interactions or a combination thereof.

In some embodiments, the diagnostic methods disclosed herein are applicable for the diagnosis of neural tube defects and/or genetic abnormalities in a fetus.

The terms chromosomal abnormalities or genetic abnormalities as used herein refer to changes in the normal structure or number of chromosomes in cells, which can lead to developmental and health issues. These abnormalities can be classified into several types, specifically, aneuploidy and structural abnormalities, each with specific characteristics and potential impacts. More specifically, Aneuploidy, relate to the presence of an extra chromosome or the absence of a chromosome. Examples include trisomy and monosomy. More specifically, Trisomy, having three copies of a chromosome instead of the usual two. Common trisomies include Trisomy 21 (Down syndrome), Trisomy 18 (Edwards syndrome), and Trisomy 13 (Patau syndrome). Monosomy, having only one copy of a chromosome instead of two, for example, Turner syndrome (Monosomy X), where an individual has only one X chromosome.

Structural abnormalities, involve changes in the structure of chromosomes, and include deletions, where a portion of the chromosome is missing or deleted (e.g., Cri-du-chat syndrome, caused by a deletion on chromosome 5); duplications, where a portion of the chromosome is duplicated, resulting in extra genetic material; translocations, where segment of one chromosome is transferred to another chromosome; inversions, where a chromosome segment breaks off, flips around, and reattaches, changing the order of the genes, and rings, where a chromosome forms a ring structure due to deletions in telomeres, causing the ends to fuse.

Another aspect of the present disclosure is a screening method for identifying a compound that modulates the interaction of an affinity moiety with a target molecule in at least one sample. The method comprising: (i) contacting the at least one sample with a bio-transistor system, in the presence and the absence of at least one candidate compound. The bio-transistor having an active region (also referred to herein in some embodiments as a modified active region) carrying selected at least one type of affinity moieties; (ii) performing one or more measurements for each sample, each measurement comprising: applying a selected electric potential on the sample, and determining current transmission profile through a channel of the bio-transistor with respect to potential variation of at least one gate electrode of the bio-transistor; and (iii) processing data on the current transmission through the channel for one or more selected gate potential and one or more selected electric potential values applied on the sample; and (iv) determining presence and/or quantity of the one or more target molecules in accordance with pre-stored calibration data, thereby determining a target molecule value for the sample in the presence of the candidate compound, and a target molecule value in the absence of the candidate compound; and (v) determining that the candidate compound is a modulator of the interaction between the affinity moiety and the target molecule, if the target molecule value obtained for the sample in the presence of the candidate compound is different from the target molecule value obtained in the absence of the candidate compound.

In some embodiments, the bio-transistor system is as defined above.

In some embodiments of the present disclosure, the disclosed screening method is particularly applicable for screening of a compound that inhibits the interaction of the affinity moiety with the target molecule. More specifically, a compound that “inhibits the interaction” as used herein, refers to a compound that reduces, prevents, blocks, impedes, suppresses, prevents, hinders and/or restricts interaction between the at least one affinity moiety and the target molecule either partially, or completely. For example, any inhibition of between about 10-50%, 51-80%, 81-99% or even, of 100% of the interaction or binding between the affinity moiety and the target molecule.

In some aspects thereof, the present disclosure further provides personalized therapeutic methods that comprise a diagnostic step using the bio-transistor systems and methods of the present disclosure, followed by administration of a therapeutic compound, for example, any compound that inhibits damage caused by acute or chronic exposure to at least one organophosphate compound.

It is to be understood that the terms “treat”, “treating”, “treatment” or forms thereof, as used herein in accordance with the personalized therapeutic methods, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the methods according to the present disclosure, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the present disclosure described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the present disclosure.

Another aspect of the present disclosure relates to a diagnostic kit. The diagnostic kit comprising: (a) at least one bio-transistor system, and optionally, at least on of: (b) at least one control sample; and (c) at least one therapeutic agent. The bio-transistor system comprises at least one transistor unit and a control system. The transistor unit comprising: (i) at least one channel; (ii) source and drain electrodes; (iii) at least one gate electrode; (iv) at least one active region located in proximity to the channel region and carrying at least one affinity moiety. It should be understood that each affinity moiety is specific for a target molecule. The at least one active region is configured for accepting at least one sample; and (v) at least one additional electrode positioned to be in electrical contact with the sample. The control system comprising at least one processor and memory circuitry. The control system is configured and operable for performing one or more measurements of the sample, wherein each measurement comprises maintaining a selected electric potential on the at least one additional electrode and determining current transmission profile through the at least one channel with respect to potential variation of said the least one gate electrode. The control unit thereby configured to determine on the presence and/or quantity of one or more target molecules in the sample.

In some embodiments, the kit is adapted for performing any of the methods of the present disclosure as described herein above.

In some embodiments, the kit provided by the present disclosure is adapted for performing a method for the diagnosis of organophosphate/s contamination in a media and/or habitat and/or for the diagnosis of a pathological condition in a subject caused by exposure to a media and/or habitat contaminated by the organophosphate/s. According to such embodiments, the bio-transistor system provided with the disclosed kits comprise as the at least one affinity moiety at least one receptor for at least one organophosphate, specifically, HFIP that is a receptor molecule for the organophosphate DCNP.

In yet some further embodiments, the present disclosure provides a kit adapted for performing a method for the diagnosis of neural tube defects and/or genetic abnormalities in a fetus.

According to such embodiments, the bio-transistor system provided with the disclosed kits comprise as the at least one affinity moiety at least one antibody that specifically recognizes at least one maternal serum marker, for example, estriol. In yet some further embodiments, the disclosed kits may comprise at least one bio-transistor systems that comprise at least one affinity moiety specific for at least one additional maternal serum marker. For example, a kit of the present disclosure may comprise according to some embodiments, bio-transistor systems comprising affinity moiety for estriol, and bio-transistor system comprising at least one affinity moiety specific for AFP and/or at least one affinity moiety specific for hCG, specifically, anti-AFP antibodies, and/or anti-hCG antibodies.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Thus, as used herein the term “about” refers to +10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, and/or parts, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

### Experimental Procedures

Fabrication of FET device. The silicon FET device is realized with a silicon-on-insulator (SOI) technology. The thickness of the SOI device layer in 145 nm and the thickness of the buried oxide is 400 nm. The n-type device layer is doped with 10¹⁷ cm⁻³, the lateral p-type regions and the n-type source and drain regions are all degenerated. The distance between the lateral p-type region define the width of the channel volume, and the distance between the source and drain define the length of the channel volume (10 μm). Standard CMOS cobalt-silicide is performed for all silicon regions contacting the aluminum lines to ensure ohmic contacts with low contact resistance. The sensing area is composed of 5.5 nm thermal SiO₂. The entire chip is passivated with 1 μm SiO₂ except the sensing area and the contacting pads.

Surface modification of the sensing area with HFIP molecules (for OP). The aminophenyl-HFIP recognition layer is first developed on 1 cm×1 cm substrates of silicon decorated with 10 nm SiO₂. The samples are thoroughly cleaned using a 2-minute sonication and 1-minute rotation in ethyl acetate, acetone, 2-propanol, and deionized (DI) water, successively. The surfaces are activated using 4-minute immersion in a solution 4:1 H₂SO₄:H₂O₂ (piranha), followed by DI rinse and dried with N₂. The samples are incubated in a 0.1% v:v solution of 3-aminopropyltrimethoxysilane (APTMS) in methanol for three hours. The samples are sonicated and rinsed several times to remove excess of APTMS, and hydrolysed for 24 hours followed by 1 hour bake at 12 0 °C. The samples are immersed in 0.5% glutaraldehyde for 40 minutes and washed with a 10 mM PBS followed by drying with N₂. Finally, the samples are incubated in aminophenyl-HFIP (B24048, Thermo Scientific) solution for 12 h at 4 °C (0.1% aminophenyl-HFIP in dimethylformamide (DMF)), cleaned with 10 mM PBS solution and dried with N₂. The samples are characterized with electrochemical impedance spectroscopy (EIS) by Palmsens 4, Palmsens Inc. The EIS is performed in a three-electrode 0.8 mL electrochemical cell filled with 0.1 mM PBS pH7.4. Conductive carbon tape is applied at the backside of the substrates which function as the working electrodes. The counter electrode is made of platinum (SEC-C Pt Gauze, ALS Japan), and an Ag/AgCl reference electrode is used (RE-1BP, ALS Japan). Ellipsometer (Alpha-SE Ellipsometer, J. A. Woollam), and contact angle tensiometer (Model OCA 20, dataphysics) are also used for surface characterization. The chemical modification process described above is subsequently applied to the silicon devices to convert them into MNChem sensors.

Surface bio-chemical modifications and surface characterization (for estriol). The MNC device is modified into an MNC bioFET as the active sensing area is biofunctionalized with anti-estriol antibodies. Initially, the process is developed using 2 cm×2 cm silicon samples decorated with 10 nm SiO₂ layer. Subsequently, the same process is applied to the MNC devices. The sample is thoroughly cleaned using ethyl acetate, acetone, 2-propanol, and deionized (DI) water. The samples are treated with each of these for 2 minutes in a sonicator and rotated for 1 minute. Surface activation (the formation surface hydroxyls) is performed using a piranha solution (4:1 H₂SO₄:H₂O₂) for 4 minutes. After rinsing with DI water and drying with an N₂ gun, the sample is incubated in a 0.1% v:v solution of 3-aminopropyltrimethoxysilane (APTMS) in methanol for 3 hours. Excess APTMS is removed using sonication and rinsing with methanol in multiple cycles. The sample is transferred into DI water for 24 hours to facilitate hydrolysis to promote the extension of amine moieties away from the surface [Zhu, M., et al. Langmuir 28, 416-423 (2012]. To stabilize the covalent bonds between the silane molecules and the oxide surface, the sample is placed in an oven for 1 hour at a temperature of 120 °C. This helps to ensure the durability and stability of the bonds. The next step involves the immobilization of anti-estriol antibodies on the SiO₂ surface. The sample is immersed in a 0.5% glutaraldehyde (GA) crosslinker solution, providing an anchor for the antibodies. Afterward, the sample is incubated for 12 hours in a solution containing 1 μg/ml of anti-estriol (ab150407, Abcam) in 10 mM TBS buffer at pH 7.4, maintaining a temperature of 4 °C. This strategy is based on the known principle that antibodies are typically immobilized to aldehyde-terminated surfaces by the reaction between the amine moieties of the antibody and the aldehyde group. The different biofunctionalization steps are characterised by spectroscopic ellipsometry (Alpha-SE Ellipsometer, J. A. Woollam), electrochemical impedance spectroscopy (EIS) (Palmsens 4, Palmsens Inc.), and contact angle tensiometer (Model OCA 20, dataphysics). The EIS measurements are conducted in a 0.1 mM PBS buffer, pH of 7.4. The sample is positioned within a three-electrode electrochemical cell (0.8 mL). The back side of the silicon working electrode is scratched and contacted with conductive carbon paint. A Pt wire (SEC-C Pt Gauze working electrode, ALS Japan) serves as the counter electrode, and an Ag/AgCl electrode serves as the reference electrode (RE-1BP, ALS Japan). The same biofunctionalization steps are applied for the biofunctionalization of the MNC device.

Formation of the biorecognition layer composed of anti-AFP antibodies. The MNC decorated with devices were biofunctionalized and transformed into MNC biosensors. The biofunctionalization was first developed on plain silicon samples (2×2 cm) 5 nm of SiO₂. The various involved biofunctionalization steps were characterized by electrochemical impedance spectroscopy (EIS) (Palmsens 4, Palmsens Inc.), contact angle tensiometer (Model OCA 20, dataphysics), and spectroscopic ellipsometry (J. A. Woollam Alpha-SE Ellipsometer). The samples were successively cleaned with ethyl acetate, acetone, and 2-propanol for 2 min in a bath sonicator for each cleaning step. The cleaning was followed by surface activation with piranha for 4 minutes (4:1 ratio of H₂SO₄:H₂O₂), DI water wash and N₂ drying. The next step was surface chemical modification with 3-aminopropyltrimethoxysilane (APTMS) linker molecules. The samples were incubated in APTMS solution (0.1% v:v in methanol) for 3 hours, followed by three cycles of 3 minutes each of sonication in methanol. The samples were hydrolyzed by 24 hours incubation in DI water, dried and placed in an oven for 1 hour at 120 °C. [M. Zhu, M. Z. Lerum, W. Chen, Langmuir 2012, 28, 416-423. DOI 10.1021/la203638g]. Prior to the binding of the anti-AFP antibodies, the APTMS was modified with glutaraldehyde (GA) (SAB4501531-100UG, Sigma Aldrich). Finally, the samples were incubated for 12 hours in 1 μg/ml solution (10 mM PBS, pH 7.4) of anti-AFP (Abcam, AB-ab3980). The MNC chips were biofunctionalized in a similar manner. The various involved biofunctionalization steps were characterized by electrochemical impedance spectroscopy (EIS) (Palmsens 4, Palmsens Inc.), contact angle tensiometry (Model OCA 20, dataphysics), and spectroscopic ellipsometry (J. A. Woollam Alpha-SE ellipsometer). The contact angle of anti-AFP modified chip was 52°+0.5° and the measured thickness of the anti-AFP layer was 2.2+0.8 nm.

Electrical measurements. The electrical measurements are performed on a probe station where the MNChem electrical pads are contacted with needles secured to micro-positioners connected to source measuring units (SMUs) of the B1500 Semiconductor Parametric Analyzer (Keysight Ltd.). 0.5 μL drops of 0.1 mM PBS pH7.4 are drop cast on the MNChem. The drop potential is set by an Ag/Ag⁺ quasi-reference electrode formed from a commercial reference electrode (012171 RE-7, ALS Co., Ltd) where the glass holder is removed and the Ag wire is painted with an Ag/AgCl ink (011464, ALS Co., Ltd). The quasi-reference electrode is secured to a micro-positioner and electrically connected to an additional B1500 SMU. The sensing measurements are performed with respect to the DCNP target molecule (CAS no. 2942-58-7, TCI).

### Electrical Measurements in Neutral Solutions (for Estriol).

The electrical measurements of the MNC devices are performed on a probe station. The chip metal pads are contacted with probe needles to establish connections with the source measuring units (SMUs) (B1500 semiconductor parameter analyzer, Keysight Ltd). A pipette is used to manually introduce a 0.5 μL drop of 0.1 mM pH 7.4 phosphate-buffered saline (PBS) to the MNC sensing area. The solution gate is realized by physically contacting the drop with a quasi-reference electrode, which comprises an Ag/Ag+ reference electrode (012171 RE-7, ALS Co., Ltd). In this configuration, the glass holder is removed to expose the Ag wire, which is coated with an Ag/AgCl ink (011464, ALS Co., Ltd). An additional B1500 SMU is connected to the quasi-reference electrode to enable potential determination of the solution (V_{GF}). The quasi-reference electrode is securely mounted on a probe manipulator, ensuring stability during the measurements. The drop maintains its integrity for a period of 2-3 minutes before evaporation, offering a sufficient time window for conducting different current-voltage (I-V) measurements. To ensure repeatability, the I-V measurements are repeated throughout the lifetime of the drop and are also performed for successive identical applied drops. The measurements in a neutral solution are exclusively carried out during the biofunctionalization process of the MNC devices. These measurements serve to validate the functionality of the devices after modification. The remarkable repeatability observed in both unmodified MNC devices and biofunctionalized MNC biosensors underscores the buffer capacity of the small solution drops. Moreover, it demonstrates the stability of the quasi-reference electrode, accounting for possible variations between successive drops.

### Sensing Measurements (for Estriol).

Sensing measurements commence with the preparation of a 0.5 μL drop of 1:100 diluted plasma with a Tris-buffered saline (TBS) 0.1 mM pH 7.4 solution. The plasma samples are obtained from the Kaplan Medical Center blood bank. In the subsequent step, the 1:100 diluted plasma is spiked with estriol molecules (E1253, Sigma-Aldrich). However, owing to the limited solubility of estriol in TBS, a two-step process is employed. Initially, estriol is dissolved in ethanol, where it exhibits solubility, and afterwards this estriol solution is added to the diluted plasma. This approach enables the preparation of estriol concentrations ranging from 1 fg/ml to 10 μg/ml. Importantly, the final solutions all contain the same amount of ethanol (1%).

The 1:100 diluted plasma drop is measured as described earlier for the neutral solution drop. To establish and validate repeatability, a series of successive I-V measurements are performed for each drop. After completing the measurements, the 1:100 diluted plasma drop is carefully collected using a clean-room wipe. To proceed with the next set of measurements, a fresh 1:100 diluted plasma drop with a higher estriol concentration is introduced onto the MNC sensing area, and the same measurement procedure is repeated. This process is iterated to ensure reliable data collection for various estriol concentrations. The non-specific measurements involve introducing estriol to an unmodified MNC device and exposing an APTMS-modified MNC biosensor to estriol. Furthermore, MNC biosensors modified with anti-estriol antibodies are subjected to the introduction of estrone (E9750, Sigma-Aldrich) and estradiol (E1024, Sigma-Aldrich), both are members of the estrogen family and differ very little from the estriol. In all non-specific measurements, the concentrations of the small molecules (estriol, estrone, and estradiol) were 10 fg/ml, 10 pg/ml, 10 ng/ml, and 10 μg/ml.

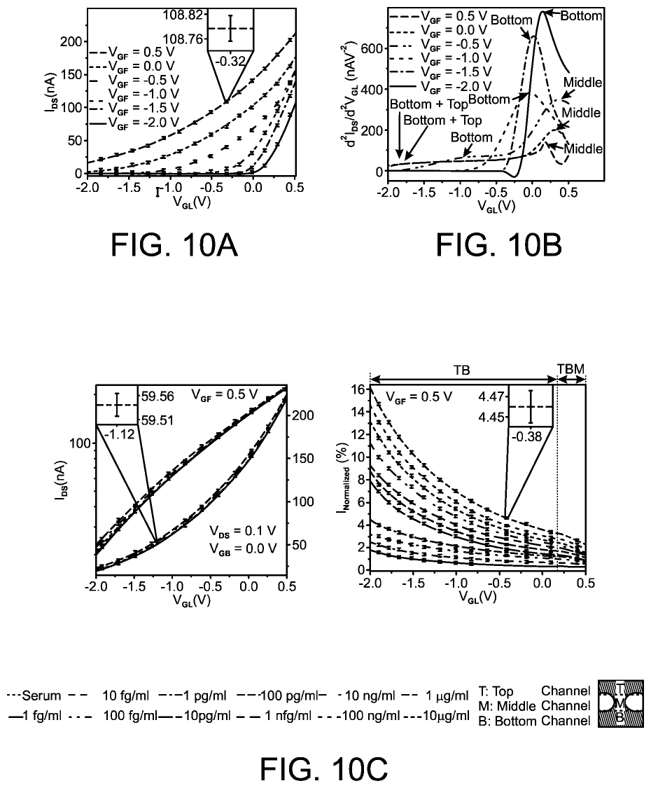

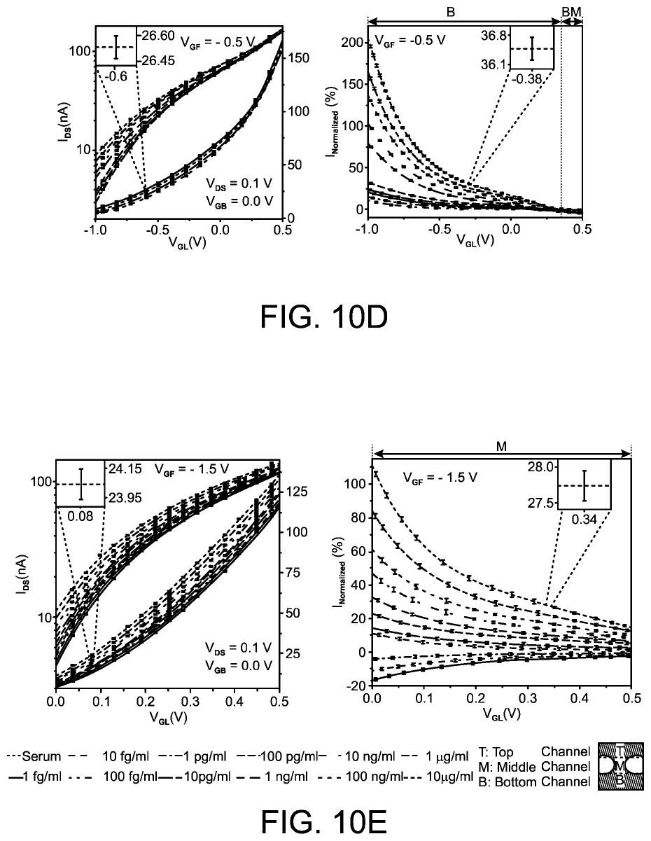

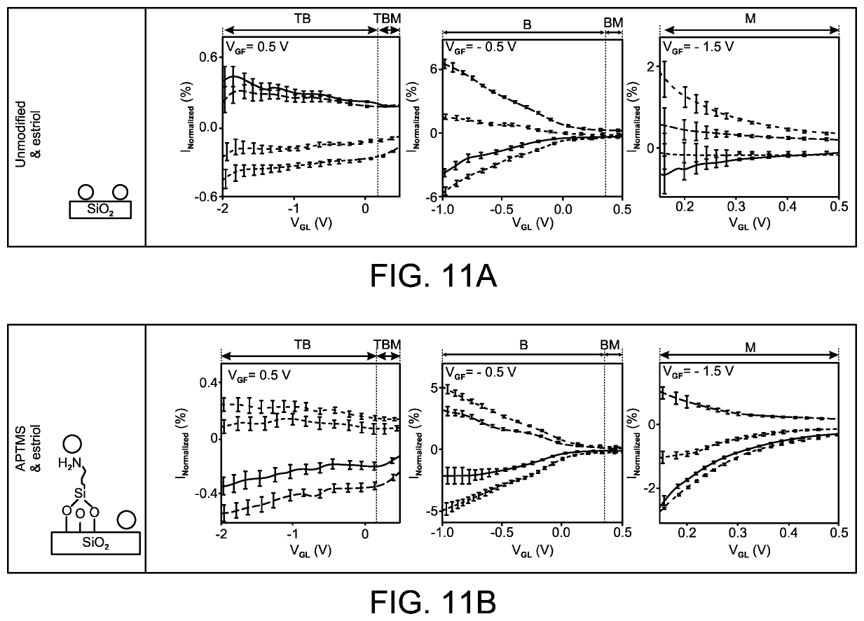

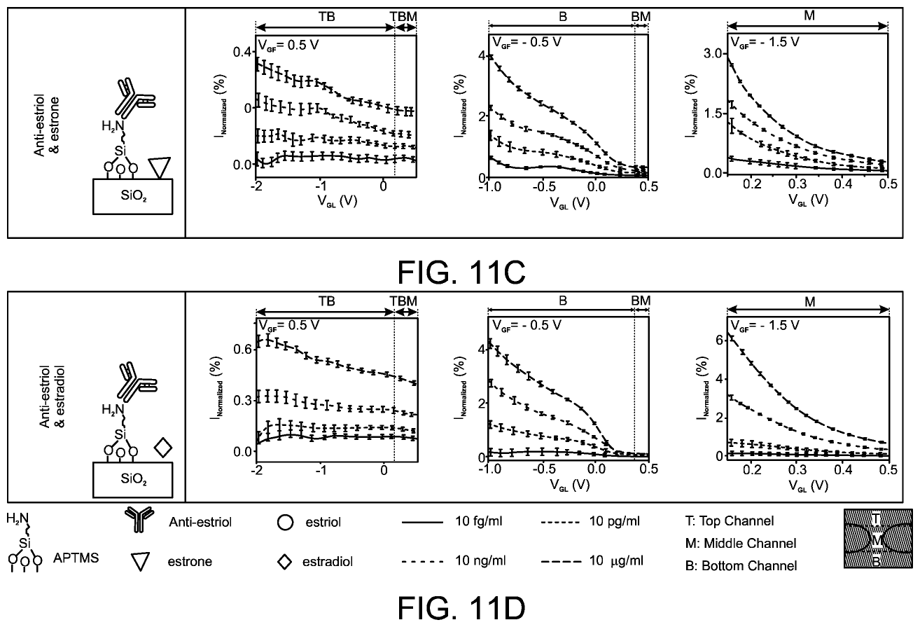

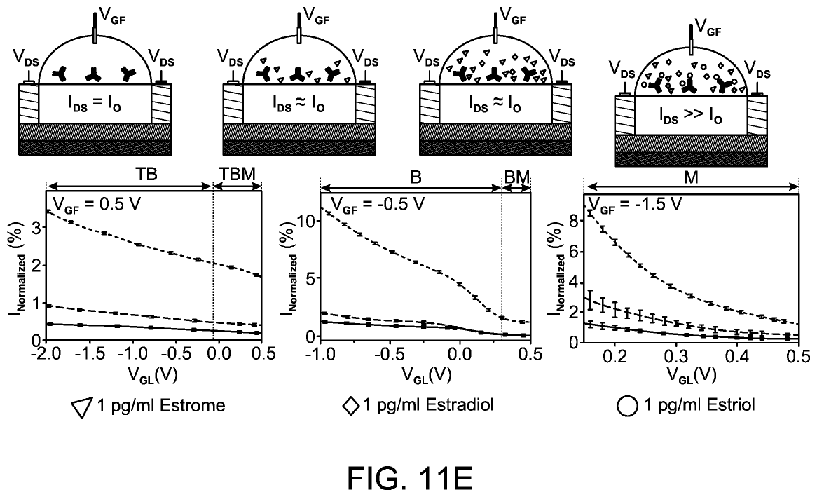

Extraction of the specific sensing performance. A population is defined as (X)+ (σ), where X is the average of the three measurements performed for each drop and σ is the corresponding standard deviation. The specific sensing performance is obtained by considering two criteria:
1) The difference between two successive drops (=concentrations) is higher than the natural variation between two identical drops of 1:100 diluted plasma of FIG. 10A. First, the average difference between successive drops, for the 17 drops in FIG. 10A, is determined. Secondly, the difference between populations of successive drops of FIG. 10C-10E is determined. Every data point in FIG. 10C-10E is considered for specific sensing if the difference between populations, of two successive drops, is higher than the corresponding average difference between the 17 drops of FIG. 10A.
2) The difference between two I_{Normalized} populations of successive drops in FIG. 10C-10E needs to be higher than the corresponding difference between two I_{Normalized} populations of successive drops in FIG. 11A-11E (non-specific measurements). As the measurements in FIG. 11A-11E are performed for 4 representative target analyte concentrations (10 fg/ml, 10 pg/ml, 10 ng/ml, and 10 μg/ml), the I_{Normalized} population which is selected for comparison with the populations of FIG. 10C-10E is the I_{Normalized} population of the smallest target concentration which is still higher than the concentration of corresponding populations of FIG. 10C-10E.

AFP electrical and sensing measurements. The MNC devices were electrically measured on a probe station with probe needles contacting the chip metal pads to source measuring units (SMUs) of the B1500 semiconductor parameter analyzer by Keysight Ltd. A neutral solution of 0.1 mM pH 7.4 phosphate-buffered saline (PBS) with a volume of 0.5 μL was manually applied with a pipette to the MNC sensing area. The drop was electrically contacted with a quasi-reference electrode made of an Ag/Ag⁺ reference electrode (012171 RE-7, ALS Co., Ltd) where the glass holder was removed by exposing the Ag wire which was coated with an Ag/AgCl ink for reference electrode (011464, ALS Co., Ltd). The quasi-reference electrode (V_{GF}) was connected to an additional B1500 SMU for determining potential of the solution. The quasi-reference electrode was mounted on a probe manipulator. The drop lifetime before evaporation is 2-3 minutes, which provides a time window for the various current-voltage (I-V) measurements. The I-V measurements were repeated during the drop lifetime, and the measurements were performed for successive applied drops in order to confirm and establish repeatability. The measurements in neutral solution were performed solely during the process of MNC device biofunctionalization in order to validate device functionality post modification. The excellent repeatability of unmodified MNC devices and biofunctionalized MNC biosensors ensure the buffer capacity of the small solution drops [R. E. G. Van Hal, et al. Sensors Actuators B 1995, 24-25, 201], as well as the stability of the quasi-reference electrode for possible drop-to-drop variations [L. R. F. Allen J. Bard, Electrochemical Methods: Fundamentals and Applications, 2nd Edition, John Wiley And Sons Inc., 2008].

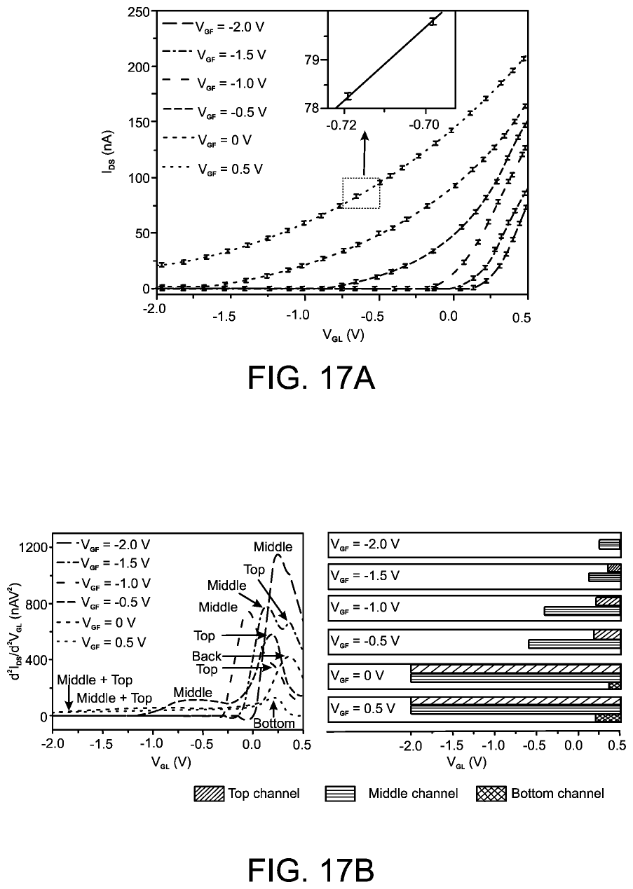

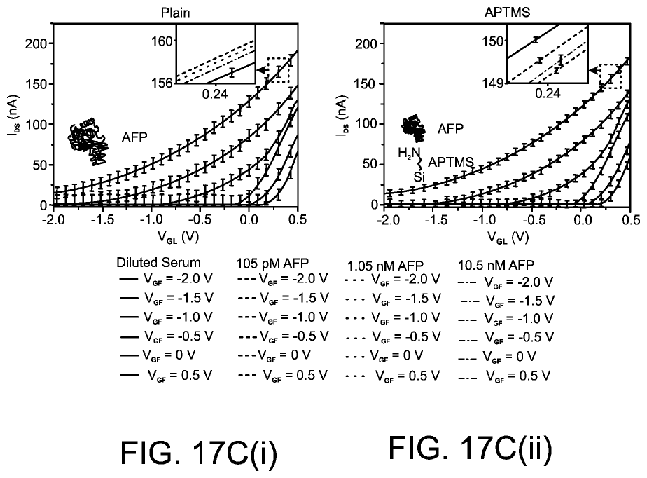

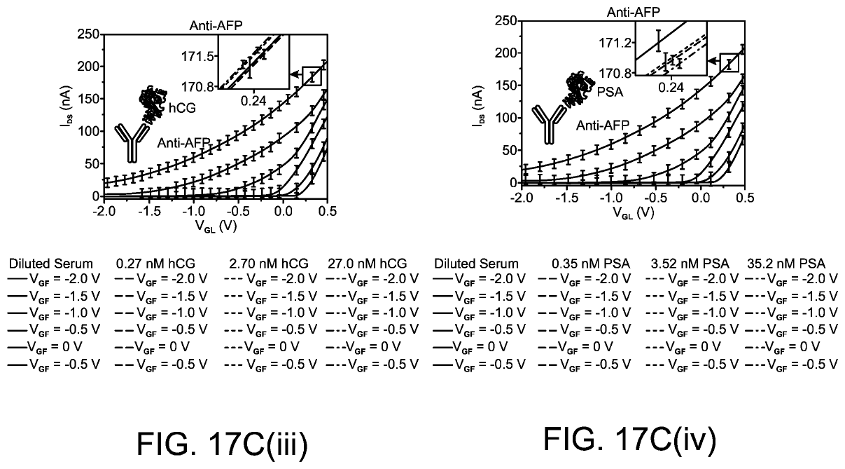

The AFP sensing measurements were performed with 0.5 μL drops of 1:100 diluted serum obtained from Kaplan Medical Center blood bank. The dilution of the serum was performed with Tris-Buffered saline (TBS) 0.1 mM pH 7.4. Mixtures of diluted serum spiked with different concentrations of AFP (Abcam, ab114216) were prepared. The serum drop was biased in the same manner described for the neutral solution drop. The stability of the quasi-reference electrode with respect to drop-to-drop variations of the diluted serum [L. R. F. Allen J. Bard, Electrochemical Methods: Fundamentals and Applications, 2nd Edition, John Wiley And Sons Inc., 2008], and non-specific adsorption of biomolecules (on the quasi-reference electrode) was addressed by the non-specific measurements (FIG. 17C). Successive I-V measurements were performed for a diluted serum drop in order to validate and establish repeatability. Afterwards, the diluted serum drop was collected with a clean-room wipe, and a new drop spiked with a higher AFP concentration is introduced on the MNC sensing area, and the procedure was repeated. The non-specific measurements were performed in the following manner: AFP was introduced to an unmodified MNC device, and AFP was introduced to an MNC modified with APTMS. Also, human chorionic gonadotropin (hCG) hormone (Sigma-Aldrich, hCG-C1063), and prostate specific antigen (PSA) (Sigma Aldrich, P3338-25UG) physiological biomarkers were introduced to MNC biosensor modified with anti-AFP antibodies. hCG was selected as a different glycoprotein type of biomarker with a dimer consisting of a 145 amino acid beta-subunit that is unique to hCG and a 92 amino acid alpha-subunit, having a molecular weight ~37 kDa. Similarly, PSA is a 237 amino-acids long single chain glycoprotein with a molecular weight of ~28 kDa. AFP is a glycoprotein which consists of a polypeptide chain with 591 amino acids and a carbohydrate chain with a molecular mass of ~68.8 kDa. For all the non-specific measurements, the concentrations of the biomarkers (AFP, PSA and hCG) were 10 ng/ml, 100 ng/ml, and 1 μg/ml corresponding with the highest AFP concentrations (equivalent to 105 pM, 1.05 nM and 10.5 nM, respectively) considered in this work for the specific measurements. The excellent repeatability of the non-specific measurements also removes the concern of pH fluctuations due to the presence of biomolecules in the drops [R. E. G. Van Hal, et al. Sensors Actuators B 1995, 24-25, 201].

### Example 1

I_{DS}-V_{GL} Curves for Selected V_{GF} Values Performed for MNC Biosensor Biofunctionalized with HFIP Receptor Molecules (for PBS Solution)

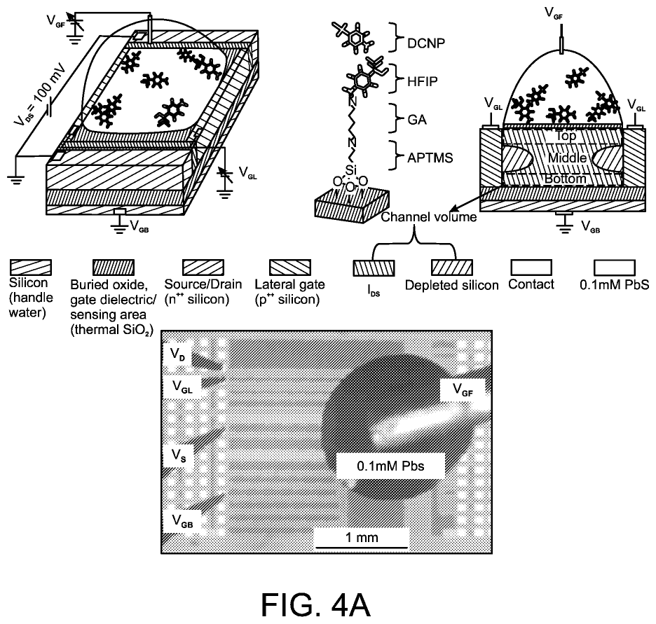

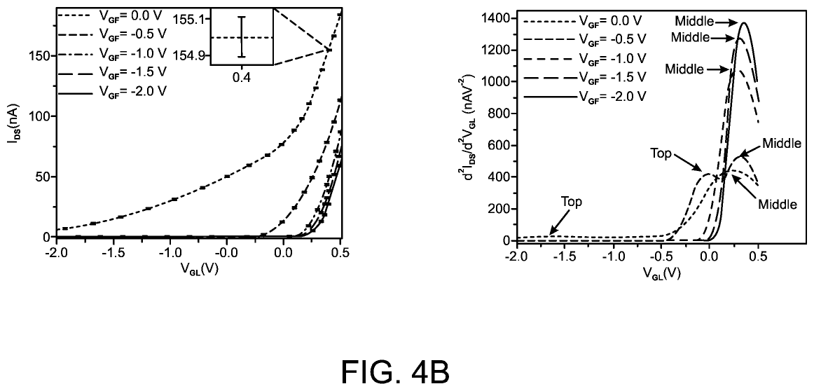

FIG. 4A presents the suggested MNChem sensor. Details of the MNChem are provided in the experimental procedures section. Briefly, the MNChem sensing methodology is based on electrostatic management of both the MNChem solid phase and solution phase to maximize the coupling between the chemical events and I_{DS}, as was already demonstrated for biosensing [8]. This approach is enabled by the presence of multiple gates. I_{DS} is gated by four gates: backgate (V_{GB}) which is grounded in the current work, two lateral gates (V_{GL}), and a front solution gate (quasi-reference electrode) which also governs the solution potential (V_{GF}). The MNChem conducting channel (I_{DS}) is composed of electrons majority carriers present in the channel volume (FIG. 4A). The width of the channel is determined by p-n junctions serving as lateral gates (V_{GL}); reverse biasing of the junctions increases the depletions inside the channel volume and therefore decreases the width of I_{DS}. The solution potential is determined by the quasi-reference electrode (V_{GF}) which also affects the top region of I_{DS}. The substrate is grounded throughout the current study.

In the present disclosure, I_{DS}-V_{GL} curves are measured for various V_{GF} values. Such measurements allow the excitations of various channel configurations, differing in shape, size, and location within the channel volume, as well as allowing for different conditions at the sensing area double layer as determined by V_{GF} (i.e. different distributions of ion concentration pH level and electric field) [8]. In general, the channel volume is composed of three main channels: top, middle and bottom, as illustrated in the cross-section of FIG. 4A. The excitation and the size of each channel depends on the respective gating conditions. FIG. 4A also schematically illustrates the process of surface chemical modifications performed for the tethering of the aminophenyl-HFIP receptor molecules on the MNChem sensing area. Briefly, the sensing area is activated with piranha, (3-Aminopropyl) trimethoxysilane (APTMS) molecules are tethered to the surface, and the aminophenyl-HFIP receptor molecules are tethered to the APTMS molecules with a glutaraldehyde (GA) linker.

The different biofunctionalization steps are characterised by spectroscopic ellipsometry (Alpha-SE Ellipsometer, J. A. Woollam), electrochemical impedance spectroscopy (EIS) (Palmsens 4, Palmsens Inc.), and contact angle tensiometer (Model OCA 20, dataphysics). The EIS measurements are conducted in a 0.1 mM PBS buffer, pH of 7.4. The thickness of each layer is measured by ellipsometry and is fixed for the measurement of the successive layer. The mean square error (MSE) is presented as well. The SiO₂ thickness is ~6 nm (Jaw model), per fabrication. The APTMS layer is ~1 nm (B-spline model), a bit higher than the expected APTMS monolayer for an APTMS molecule length of ~5 Å chain length, with 1 Å per bond. The HFIP thickness is ~1 nm (B-spline model). The contact angle is measured at three locations for each sample, two measurements at each location, with a total of 6 measurements. The measured contact angle value for SiO₂ is 59.08°±0.73°, for piranha-treated surfaces is 24.5°±3.43°, APTMS 35.85°±0.42° and HFIP 65.14°±3.07°. A clear shift toward hydrophilicity is measured post activation and contact angle values for APTMS and receptor molecules are consistent with previous reports. EIS measurements are performed in a 10 mM pH 7.4 PBS solution for unmodified SiO₂ (SiO₂), SiO₂ modified with APTMS post hydrolysis (APTMS), and SiO₂ biofunctionalized with HFIP.

FIG. 4B presents I_{DS}-V_{GL} for various values of V_{GF} for MNChem modified with aminophenyl-HFIP (aminophenyl-HFIP-MNChem). The measurements are performed in 0.5 μl drops of 0.1 mM PBS pH 7.4. Each data point is the average of 30 data measurements (10 drops, each drop measured 3 times) and the error bars are the corresponding standard deviations (see inset). The excellent repeatability of the aminophenyl-HFIP-MNChem ensures stability of both the sensing area and the quasi-reference electrode (V_{GF}) with respect to drop-to-drop pH and potential variations.

FIG. 4B also presents the corresponding extracted 2nd derivatives of the I_{DS}-V_{GL} curves where each peak reflects the excitation of a channel [7]. Note that the bottom channel is not excited for the selected range of V_{GF} and V_{GL} values. Hence, I_{DS} persists either as a middle channel, top channel, or combination of both. Finally, the excitation voltage (threshold voltage) is higher for lower V_{GL} and V_{GF} values, and each combination of these determines a unique I_{DS} of a distinct shape, size and location in the channel volume.

### Example 2

I_{DS}-V_{GL} Curves for Selected V_{GF} Values Performed for MNC Biosensor Biofunctionalized with HFIP Receptor Molecules (for PBS Solution Spiked with DCNP)

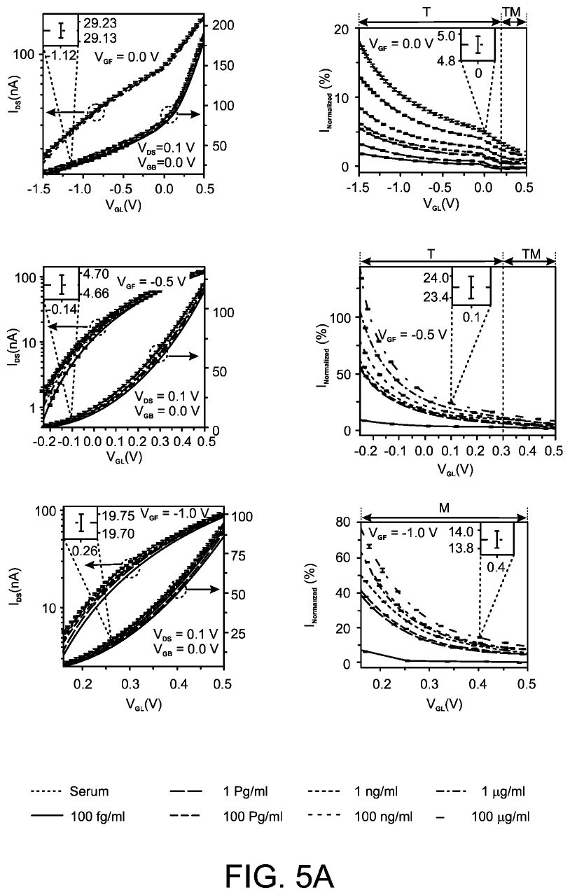

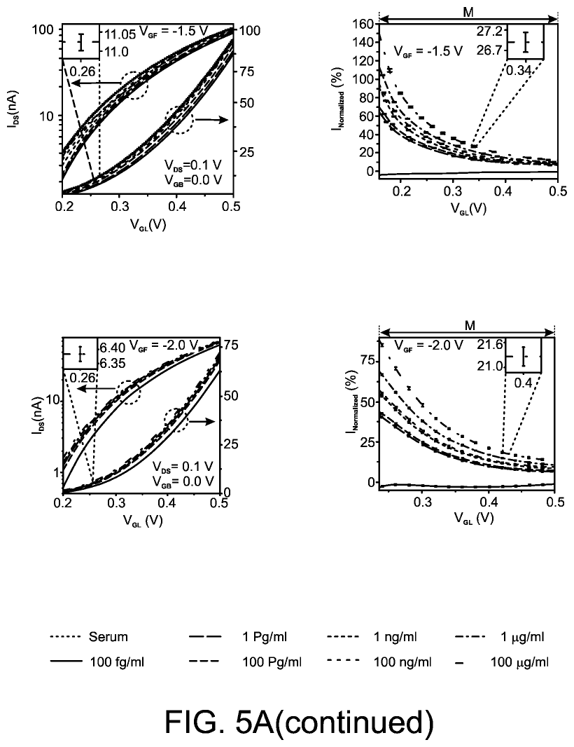

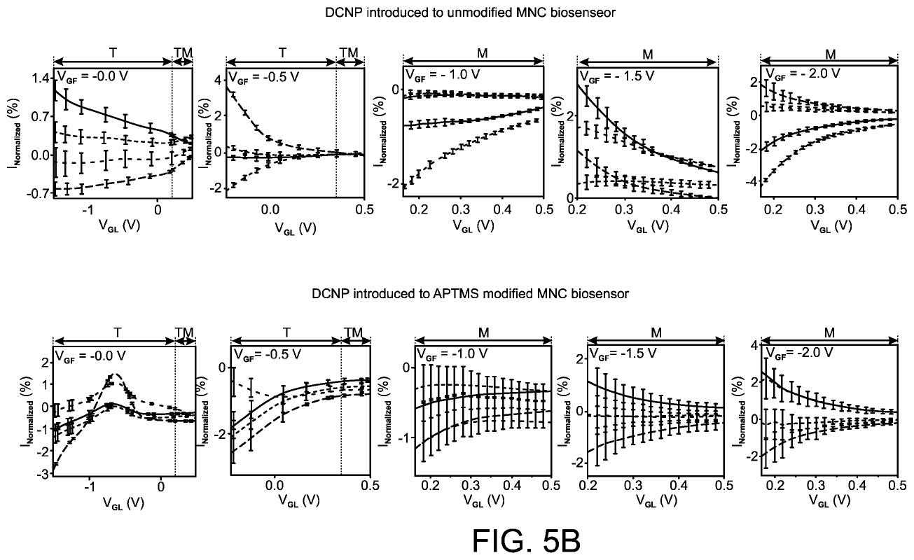

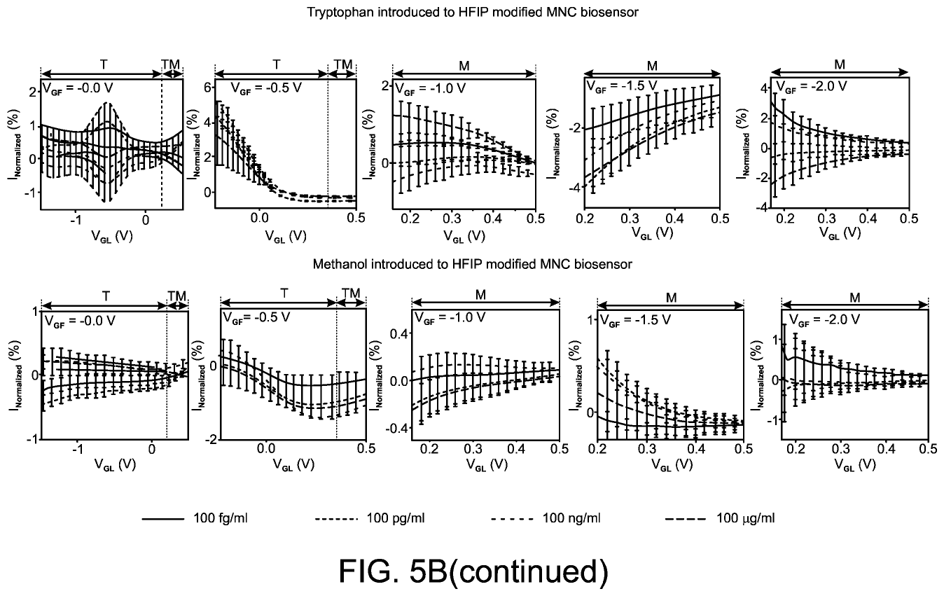

FIG. 5A presents I_{DS}-V_{GL} curves, for various V_{GF} values, following the introduction of Diethyl cyanophosphonate (DCNP) target molecules to the aminophenyl-HFIP-MNChem. The sensing is performed in 0.5 μL drops of 0.1 mM PBS solution spiked with DCNP concentrations ranging from 100 fg/ml to 100 μg/ml. Each drop contains a different DCNP concentration, and the application performed from the lowest to highest DCNP concentrations. Each data point reflects the average of three measurements performed on the same drop, and the error bars are the respective standard deviations (see insets). Also, the respective I_{Normalized} values are presented, where I_{Normalized}=(I_{DCNP}−I_{PBS})/I_{PBS}, where I_{DCNP} is I_{DS} measured for a certain DCNP concentration and I_{PBS} is the baseline I_{DS} measured for PBS containing no DCNP. The monotonous increase of I_{DS} and I_{Normalized} with increasing DCNP concentrations is evident and reflects the overall accumulation of positive charge at the aminophenyl-HFIP-MNChem sensing area upon binding of the DCNP. The channel configuration, following FIG. 4B, is also indicated at the top of the I_{Normalized} curves which reflect the dependency of I_{Normalized} on the selection of V_{GL} and V_{GF} values.

FIG. 5B presents the control measurements performed to quantify the non-specific signal. To this end four sets of measurements are performed: 1) the introduction of DCNP to an unmodified MNChem, 2) the introduction of DCNP to APTMS-modified MNChem, 3) the introduction of methanol (MeOH) aminophenyl-HFIP-MNChem, and 4) the introduction of tryptophan to aminophenyl-HFIP-MNChem. Both MeOH and tryptophan are polar and can bind the aminophenyl-HFIP layer via hydrogen bonding. As the non-specific response of the above is very limited and not visible in the I_{DS}-V_{GL} curves, only the respective I_{Normalized} curves are provided for the I_{DS}-V_{GL} curves and I_{Normalized} vs. target molecule concentration.

Note the limited non-monotonous I_{Normalized} response in FIG. 5B in comparison with the I_{Normalized} response of FIG. 5A.

### Example 3

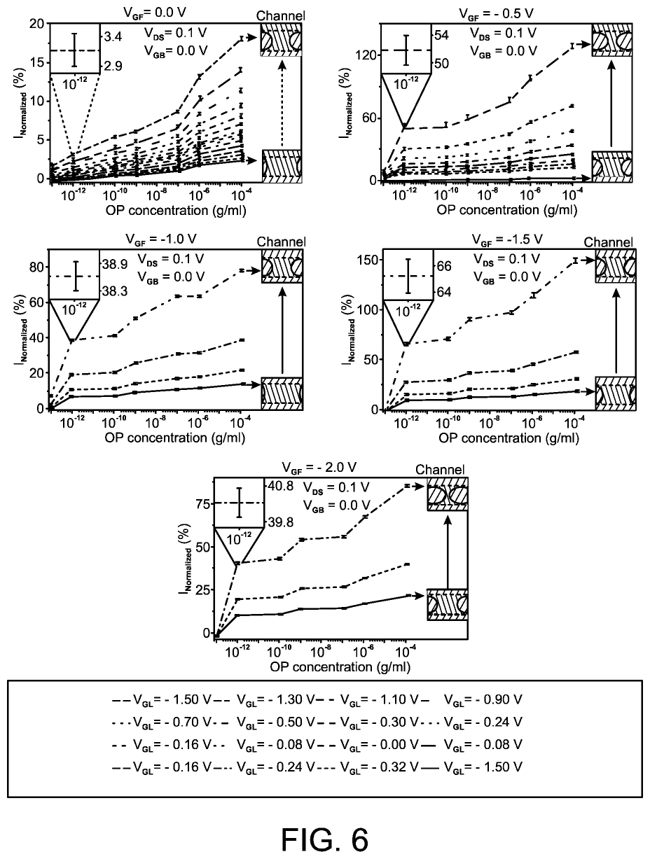

FIG. 6 presents calibration curves, that is the dependency of I_{Normalized} on DCNP concentrations for the selected V_{GL} and V_{GF} values. The calibration curves reflect the specific and label-free sensing of DCNP as the non-specific measurements are quantitively accounted for in the following manner: A population of measurements performed for a single drop at a given V_{GF} and V_{GL} values is represented by (Av.)±(σ), where Av. is the average of the drop measurements and σ is the standard deviation. Specific sensing is concluded once two criteria are satisfied: 1) The difference between successive drops of FIG. 5A must be greater than the average difference between the identical PBS drops of FIG. 4B. 2) The difference between successive drops of FIG. 5A must be greater than the difference between the respective drops of FIG. 5B (control non-specific measurements). The measurements of FIG. 5B are performed for four concentrations of target molecules (100 fg/ml, 100 pg/ml, 100 ng/ml, and 100 μg/ml). The selected concentrations of FIG. 5B for the comparison with FIG. 5A are the smallest ones which are still higher than the relevant FIG. 5A concentrations.

The calibration curves reflect the importance of tailoring the channel configuration to the electrostatics of the chemical complexes at the sensing area. Evidently, various channel configurations (different V_{GL} and V_{GF} values) present very little response toward DCNP, with almost ‘flat’ calibration curves, while selected channels show strong response towards DCNP. Importantly, note the effect of V_{GF}, for example, for DCNP concentration of 10⁻⁴ g/ml and for V_{GL}=0.16 V, I_{Normalized}=4 and 150 for V_{GF}=0 V and V_{GF}=−1.5V, respectively. V_{GF} governs both channel configuration as well as the conditions at the sensing area double layer in terms of pH, ion concentration and electric field distributions [Bhattacharyya et al. ACS Sens 5, (2020]. However, the dependency of hydrogen bonding on pH, ion concentration and electric field, for values characteristic of solid-solution double layer, are reported to be negligible [Bhattacharyya et al. ACS Sens 5, (2020)].

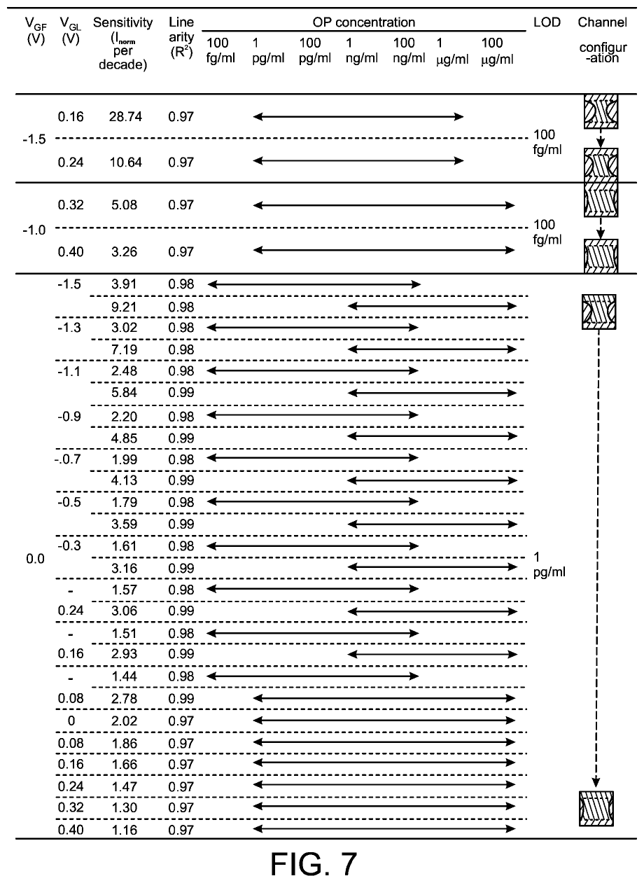

FIG. 7 discloses Table 1 that shows a summary for the sensing performance of the proposed aminophenyl-HFIP-MNChem towards DCNP. Only channels (determined by V_{GL}, V_{FG} values) with linearity equal to or greater than 0.97 and a dynamic range equal to or greater than 8 orders of magnitude, in DCNP concentrations, are selected for presentation. The performance is concluded with an LOD of 100 fg/ml, a dynamic range of 9-10 orders of magnitude and with excellent linearity and sensitivity.

The current work demonstrates specific and label-free sensing of DCNP in 0.5 μL drops of buffer solution with the MNChem modified with aminophenyl-HFIP. The MNChem provides excellent sensing performance as compared with the state-of-the-art. Moreover, the MNChem is fabricated in a CMOS process implying potential integration of various CMOS technologies on a single chip (embedded) providing possibilities for independent field-deployed units for OP monitoring. Hence, the MNChem allows a unique platform for electrochemical sensing performed in ultra-small volumes for real-time sensing. Finally, note that the reported measurements in the current work are performed without the customarily washing of the non-specific signal, and the control measurements indicate that the contribution of the non-specific signal is entirely negligible compared with the specific signal. This strongly indicates that only target molecules which are anchored to the sensing area contribute to the sensing signal, whereas the contribution of the non-specific molecules is entirely negligible.

### Example 4

I_{DS}-V_{GL} Curves for Selected V_{GF} Values Performed for MNC Biosensor Biofunctionalized with Anti-Estriol Antibodies (Background Measurements)

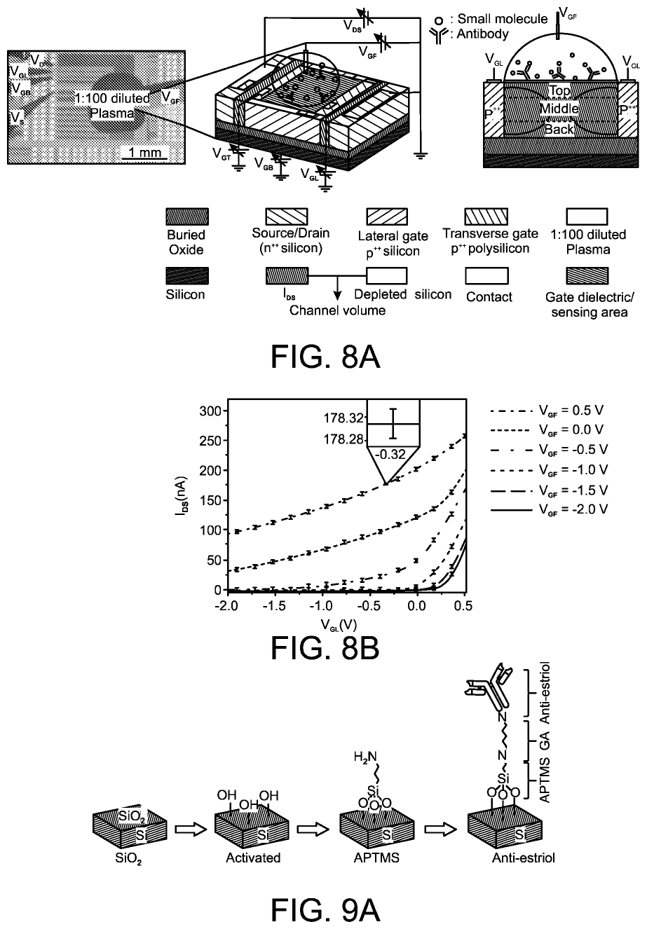

FIG. 8A shows an optical image of the MNC biochip, a three-dimensional (3D) illustration of the MNC bioFET, and an MNC bioFET cross-section midway between source and drain. The optical image shows the needle-probe contacting strategy, and the electrical contacting of the 1:100 diluted plasma drop with a quasi-reference electrode. The MNC bioFET source-drain current (I_{DS}) is composed of electron majority carriers running between the source and drain (V_{DS}). The bioFET is equipped with six gates. The front gate determines the solution potential (V_{GF}), and two lateral gates which are equally biased in this study (V_{GL}1=V_{GL}2=V_{GL}). The backgate (V_{GB}) and the two transverse gates (V_{GT1}, V_{GT2}) are not employed in this work. The MNC bioFET is protected by a SiO₂ passivation layer except for the sensing area and the electrical pads. Every combination of V_{GF} and V_{GL} results in the formation of different depletion areas within the channel volume, determining I_{DS} shape, size, and location, as shown in the cross-section. Therefore, unlike the conventional MOSFET (metal-oxide-silicon FET), where gating predominately impacts the inversion layer carrier density, the MNC bioFET defines a distinct I_{DS} shape, size, and location for each data point in an I_{DS}-V_{GL} curve. In general, the channel volume accommodates three distinct channels: the back channel, middle channel, and top channel (FIG. 8A cross-section); the shape, size and location of each channel depends on the gating configuration. The primary motivation for the MNC bioFET is to tailor I_{DS} to optimally couple with the unknown distributions of receptor-target molecule complexes at the sensing area. The MNC bioFET design enables the effective translation of non-uniform sensing area potential, induced by biological complexes, into above noise level variations in I_{DS}.

FIG. 8B shows I_{DS}-V_{GL} curves for various V_{GF} values. The measurements are conducted for 0.5 μL drops of 1:100 diluted plasma using an unmodified MNC device. Each curve represents an average of 17 drops, with three measurements performed for each drop, resulting in a total of 51 measurements (see Experiments procedures section). The inset provides a closer view of a single data point, where an error bar reflects the standard deviation of 51 measurements (the error bars are not visible in the I_{DS}-V_{GL} curves as the distributions of measurements are very tight). The curves reveal the outstanding repeatability and robustness of an unmodified MNC device. The repeatability alleviates concerns regarding: 1) drop-to-drop pH fluctuations: the measurements are performed in ambient, and the presence of the user can potentially affect the pH of the drop due to exhalation of CO₂, for example [B. M. Lowe et al, Analyst 2017, 142, 4173]. Such an effect could result in drop-to-drop variations and lack of repeatability. 2) The measurements are performed in 1:100 diluted plasma with a background protein concentration (primarily albumin and globulins) of 600-800 μg/mL. The application of consecutive drops can result in non-specific adsorption of background proteins (surface biofouling) that will directly affect the repeatability. The excellent repeatability removes this concern. 3) Quasi-reference electrodes are more susceptible to potential variations compared with standard reference electrodes. The excellent repeatability removes the concern of quasi-reference electrode potential variations due to swapping of drops [A. J. Bard et al. Wiley, Hoboken, New Jersey, USA 2000]. It also removes the concern of potential variations due to non-specific adsorption of background proteins on the electrode surface.

### Example 5

I_{DS}-V_{GL} Curves for Unmodified MNC bioFET and MNC bioFET Modified with Anti-Estriol Antibodies

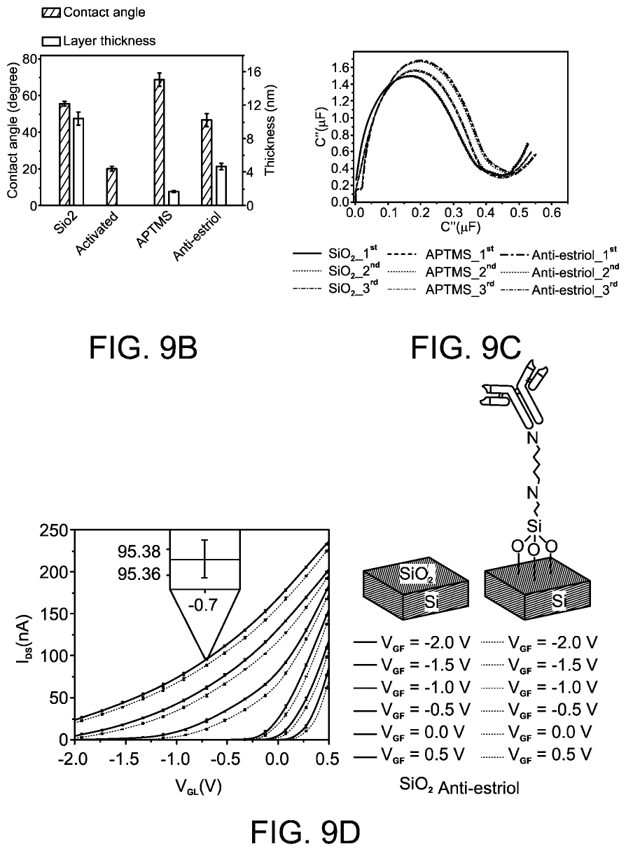

FIG. 9A presents the key steps in the biofunctionalization of the sensing area (see Experimental procedures). In brief, piranha is used for the activation of the Si/SiO₂ sensing area. The activation is followed by chemical modification with APTMS linker molecules followed by hydrolysis. The last step entails the binding of the anti-estriol antibodies to the SiO₂-bound APTMS molecules using GA linkers. The surface biofunctionalization is developed and physically characterized on 2 cm×2 cm Si/SiO₂ samples. FIG. 9B shows the contact angle and ellipsometry measurements performed for unmodified SiO₂ (SiO₂), post piranha activation (Activated), post APTMS modification and hydrolysis (APTMS), and post binding of the anti-estriol antibodies (anti-estriol). The contact angle of each sample is measured at four different locations, three measurements are performed at every location, amounting to a total of 12 measurements. The data points represent the average values, and the error bars signify the corresponding standard deviations.

The measured contact angle value for SiO₂ is 55.60°±1.48°, for piranha-treated surfaces is 19.9°±1.83°, APTMS 68.85°±2.96° and anti-estriol 46.61°±3.79°.

A significant shift towards hydrophilicity is measured post activation, as expected, clearly indicating the formation of hydroxyls on the surface, and contact angle values post APTMS and hydrolysis aligns with previous reports [Liu, H. et al. Applied Sciences (Switzerland) 12, (2022)]. Overall, the contact angle dependency on the different modification steps is consistent with the behavior that has been extensively documented in prior research [Armini et al. MRS Online Proceedings Library 1156, 408 (2009)]. The corresponding ellipsometry measurements are also presented in FIG. 9B. At each biofunctionalization step, the thickness of the relevant layer is determined, and is held fixed during the measurement of the successive layer. For all measurements, the mean square error (MSE) is less than 0.96. The thickness of the SiO₂ layer is about 10 nm (Jaw model), as expected. The thickness of the APTMS layer is approximately 1.65 nm (B-spine model), slightly higher than the anticipated value for a monolayer based on the length of the APTMS molecule (around 5 Å chain length, assuming 1 Å per bond) [Ron, I. et al. J Am Chem Soc 132, 4131-4140 (2010]. The thickness of the antibody layer is 4.65 nm (B-spine model) which suggests that the antibodies might be oriented toward the surface. These findings agree with previous research on amino silane layers on silica [Zhu, et al. Langmuir 28, 416-423 (2012] and antibodies [Wang et al. vol. 21 www.worldscientific.com (2009)]. FIG. 9C presents EIS measurements performed for unmodified SiO₂ (SiO₂), SiO₂ modified with APTMS post hydrolysis (APTMS), and modified with anti-estriol antibodies (anti-estriol). The curves present the relationships between the imaginary capacitance (C″) and the real capacitance (C′). The measurements are performed in 0.1 mM pH 7.4 PBS solution. For repeatability, every sample is measured, the electrodes and the solution are removed from the EIS cell, a new solution is introduced, the electrodes are reconnected, and another measurement is performed. This procedure is repeated three times for each sample (indicated as 1^{st}, 2^{nd} and 3^{rd} in the legend). The impact of the modifications is reflected by three distinct curves. Noticeably, the surface modifications lead to a gradual increase in capacitance suggesting that the mechanism behind the capacitance increase is related to the increase in the double layer dielectric constant.

FIG. 9D shows I_{DS}-V_{GL} curves for unmodified MNC bioFET and MNC bioFET modified with anti-estriol antibodies. The measurements are performed in 0.5 μl drops of 0.1 mM 7.4 pH PBS solution. The data points represent the average of 4 measured drops, where each drop is measured 4 times, removed with a clean-room wet towel, and a new drop is applied and measured. The error bars, enlarged in the inset, indicate the corresponding standard deviations. Finally, the small standard deviations demonstrate the excellent repeatability in 0.1 mM 7.4 pH PBS solution which ensures the stability of the quasi-reference electrode under possible drop-to-drop variations and pH fluctuations.

### Example 6

FIG. 10A presents I_{DS}-V_{GL} curves for various V_{GF} values of an MNC bioFET modified with anti-estriol antibodies. The measurements are conducted in 0.5 μl drops of 1:100 diluted plasma. The data points represent the average values of 17 measured drops. Each drop is measured 3 times, removed with a clean-room wet towel, and a new drop is applied and measured. The error bars, shown in the inset, indicate the corresponding standard deviations. Importantly, the biofunctionalized MNC bioFET exhibits a remarkable repeatability and robustness in 1:100 diluted plasma, thereby ensuring the stability of the MNC bioFET and the quasi-reference electrode with respect to possible variations as discussed in FIG. 8B. Finally, the I_{DS} decrease after biofunctionalization with anti-estriol antibodies indicates an overall negative charge at the double layer induced by surface reorganization upon the biofunctionalization.

FIG. 10B presents the second derivatives of the I_{DS}-V_{GL} curves presented in FIG. 10A, where a peak reflects the excitation of a channel [8]. For V_{GF}=0 and 0.5 V and for negative V_{GL}, the top and bottom channels are excited (no off-state), and the middle channel is excited with increasing V_{GL}. For negative values of V_{GF} and V_{GL}, all channels are closed, and as V_{GL} increases the bottom channel is excited first followed by the middle channel. Note how the peaks associated with the bottom and middle channels appear for higher V_{GL} as V_{GF} gets smaller (the lower is V_{GF} the more depleted is the channel volume and higher V_{GL} values are needed to trigger the channels).

Next, 0.5 μL drops of 1:100 diluted plasma spiked with estriol concentrations ranging from 1 fg/ml to 10 μg/ml are successively introduced and measured from smallest to highest estriol concentrations. Each drop (i.e., a different estriol concentration) is measured 4 times, removed with a clean-room wet towel, a new drop with one order of magnitude higher estriol concentration is applied and measured (See Experimental procedures). FIGS. 10A-10C show I_{DS}-V_{GL} curves for V_{GF}=0.5, −0.5 and −1.5 V, respectively, where each data point is the average of the 4 measurements performed for each drop, and the error bars are the corresponding standard deviations (see insets). Again, the hardly noticeable error bars reflect the excellent stability and repeatability of the biofunctionalized MNC bioFET in diluted plasma. Also, FIGS. 10C-10E present the respective normalized current (I_{Normalized}) defined as I_{Normalized}=(Iₑₛₜᵣᵢₒₗ−Iₚₗₐₛₘₐ)/Iₚₗₐₛₘₐ, where Iₑₛₜᵣᵢₒₗ is the current measured for the given voltage conditions (i.e., V_{GF} and V_{GL}) and estriol concentration, and Iₚₗₐₛₘₐ is the current measured under same voltage conditions for a diluted plasma drop not containing any estriol. Similar to the I_{DS}-V_{GL} curves, the data points of the I_{Normalized} Curves are the averages of the 4 measurements, performed for each drop, and the error bars are the respective standard deviations. Note that the channel configuration, as determined in FIG. 10B, is indicated at the top of the I_{Normalized} curves (T, B, M for top, back and middle channels).

First note, unlike the repeatability curves of FIG. 10A, the curves of FIG. 10C-10E diverge with increasing estriol concentrations. Also, I_{Normalized} depends on V_{GL}, V_{GF}, or equivocally, on channel configuration. For example, for estriol concentration of 10 μg/ml and V_{GF}=−0.5 V, I_{Normalized} ranges from ~200 for V_{GL}=−1 V and is just about nulled for V_{GL}=0.5 V, which reflects a channel configuration transition from a bottom channel to a bottom-middle channel, as indicated at the top of the figure. Finally, note, in general, an increase of I_{DS} with estriol concentration is measured which indicates an accumulation of positive charge at the solid-solution interface. However, to determine the nature of the sensing mechanism the non-specific signals are discussed next.

### Example 7

Next, measurements are performed to quantify the extent of the non-specific signal. To this end, the following measurements are performed: 1) the introduction of estriol to an unmodified MNC bioFET, 2) the introduction of estriol to an MNC bioFET modified with APTMS post hydrolysis, and 3) the introduction of other members of the estrogen family (estrone and estradiol) to MNC bioFETs biofunctionalized with anti-estriol antibodies. For all these non-specific measurements, 4 concentrations of the relevant target analyte are selected: 10 fg/ml, 10 μg/ml, 10 ng/ml and 10 μg/ml. The variations in I_{DS}-V_{GL} curves for all non-specific measurements depend very weakly on target molecule concentration, and the difference between the I_{DS}-V_{GL} curves is very difficult to discern visually. Therefore, the I_{DS}-V_{GL} curves are not provided, and alternatively, FIGS. 11A-11E present the corresponding I_{Normalized} curves for V_{GF}=0.5, −0.5, −1.5 V which provide a better visualization (additional I_{Normalized} curves for other V_{GF} were calculated but are not presented).

FIG. 11A shows I_{Normalized} curves for selected V_{GF} values for estriol introduced to an unmodified MNC bioFET. The goal of this measurement is to quantify the non-specific signal attributed to estriol molecules physically adsorbed on the sensing area and/or on the surface of the quasi-reference electrode. Two observations are in place: 1) the I_{Normalized} values are considerably smaller than the I_{Normalized} values presented in FIGS. 10C-10E, and 2) the lack of monotonous I_{DS} increase with increasing estriol concentration, in contrast with the monotonous I_{DS} increase for MNC bioFET biofunctionalized with anti-estriol antibodies (FIG. 10C-10E). A similar response is measured for the MNC bioFET modified with APTMS (FIG. 11B). Also, importantly, the small non-specific signals imply that the introduction of high concentrations of estriol to the 0.5 μL drops of 1:100 diluted plasma does not produce significant pH variations.

The next non-specific measurements involve another two members of the estrogen family: estrone and estradiol, which are presented in FIG. 11C and FIG. 11D, respectively. Both estrone (presented below as formula #2) and estradiol (presented below as formula #3) are identical with the estriol (presented below as formula #1) expect these lack a single hydroxyl group. In addition, the estrone presents a carbonyl instead of the hydroxyl of the estriol. Importantly, both estrone and estradiol are non-specific with respect to the anti-estriol antibody.

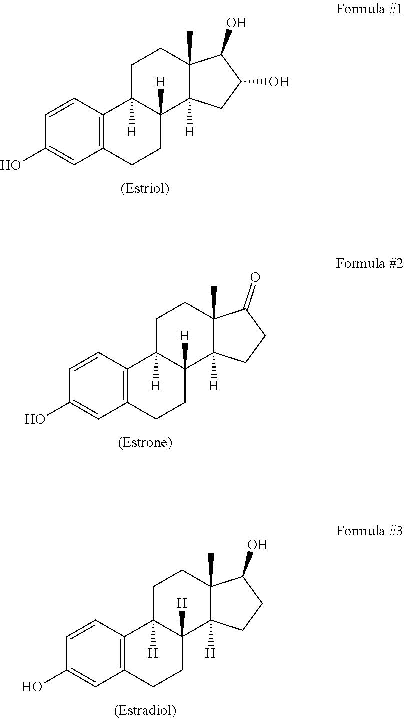

Interestingly, unlike the measurements presented in FIG. 11A and FIG. 11B a monotonous I_{Normalized} increase with target molecule increase is measured for all V_{GF} values and for both estrone and estradiol. This is unexpected as the estrone, and estradiol are non-specific towards anti-estriol. Hence, these measurements reflect the enhanced sensitivity provided by the MNC bioFET (compared with the competitive ELISA [antibody [https://www.abcam.com/products/primary-antibodies/estriol-antibody-epptxr3-ab150407.html#lb.]. Still, note that the I_{Normalized} values are orders of magnitude smaller than the values presented for estriol applied to MNC bioFET biofunctionalized with anti-estriol antibodies (FIGS. 10C-10E).

Finally, after the measurements presented in FIG. 11D are concluded, the chip is removed and thoroughly rinsed with DI water. Afterwards, three successive drops of 1:100 diluted plasma spiked with 1 μg/ml of estrone, 1 μg/ml of estradiol, and 1 μg/ml of estriol are applied in this sequence, one at a time, measured, and removed (See illustration FIG. 11E). The corresponding I_{Normalized} curves are provided in FIG. 11E where the data points are the average of four measurements performed for each drop and the error bars are the respective standard deviations. Again, note the significant higher I_{Normalized} values obtained for the estriol drop. Of course, the I_{Normalized} values are considerably smaller than the ones provided in FIG. 10C-10E as the MNC bioFET is exposed to high concentrations of non-specific molecules prior to the introduction of the estriol.

Importantly, this study of non-specific signals suggests the negligible role of non-specific interactions in bioFETs, in contrast with current ubiquitous practice of non-specific washing (following ELISA procedures). This surfaces a great research question as to the reason by which specific interactions produce variations in surface potential, whereas the contributions of non-specific interactions to surface potential are negligible.

### Example 8

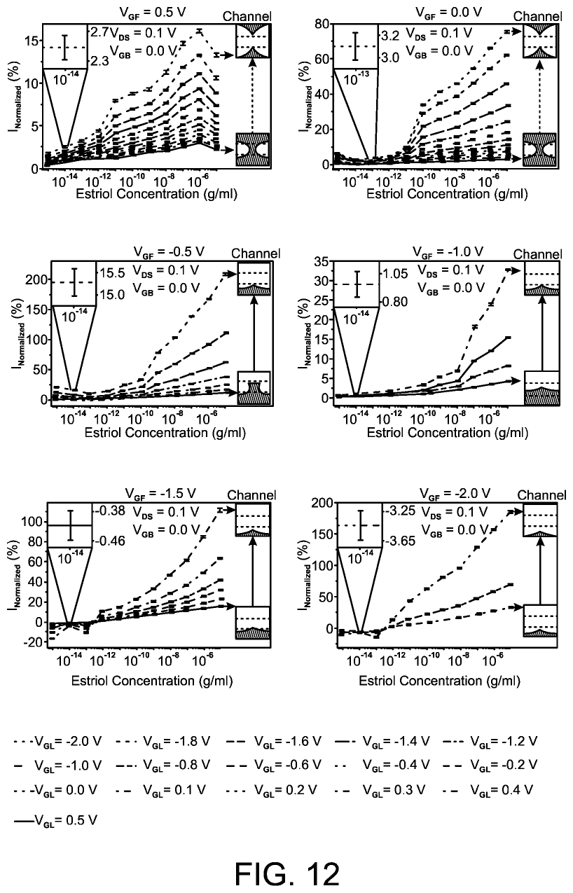

The specific and label-free estriol sensing performance of the MNC bioFET is extracted from the performed measurements. The procedure is detailed in the Experimental procedures section. Briefly, the I_{Normalized} values of FIG. 10C-10E are considered for specific sensing only if the variations between two consecutive concentrations, for each V_{GL} and V_{GF}, are higher than: 1) the natural drop-to-drop variation of FIG. 10A, and 2) each of the corresponding non-specific measurements of FIG. 11A-11E. Therefore, the extracted specific sensing performance accounts for possible drop-to-drop pH and potential variations, non-specific adsorption on the sensing area and/or the quasi-reference electrode, interactions between the anti-estriol antibodies and non-specific target molecules, pH variations due to the introduction of target molecules to the 0.5 μL drops of diluted plasma, and natural drop-to-drop variations associated with the experimental set-up. FIG. 12 presents calibration curves, I_{Normalized} vs. estriol concentrations at selected channel configurations (i.e., selected V_{GF} and V_{GL} values). The calibration curves are generated after the above steps 1 and 2 are performed, and therefore these calibration curves represent specific sensing performance. Unlike FIG. 10 and FIG. 11, the calibration curves are presented for all the V_{GF} values considered in the current work. Note that every data point in FIG. 12 is an average of all 4 measurements performed for a single estriol concentration (=drop), and the error bars reflect the corresponding standard deviation (insets). Also, illustrations are provided on the right of the calibration curves to illustrate the relevant channel configuration. Note that less channels are evident (less curves) as V_{GF} decreases, as smaller V_{GF} implies a depleted channel volume and therefore channel excitation is more limited. Clearly, I_{DS} increases with estriol concentration which suggests an overall increase of positive charge at the double layer.

Estriol sensing is challenging due to the need for high specificity and selectivity, coupled with the need to generate sufficient electrostatic perturbation notwithstanding the estriol charge neutrality and small size. As for the specificity and selectivity, a series of sterol-antibody interactions revealed that the steroid body is mostly buried within a hydrophobic pocket of the antigen-binding fragments (Fab) complementarity determining regions (CDR) [Valjakka, J. et al. Journal of Biological Chemistry 277, 44021-44027 (2002)]. For example, crystallography and modeling of estradiol-antibody binding demonstrates that binding occurs in a deep hydrophobic pocket between the two variable domains of the antibodies [Monnet, C. et al. J Mol Biol 315, 699-712 (2002)]. Aromatic residues that surround the antigen binding site contribute to ligand binding owing to their large hydrophobic surfaces and the ability to form hydrogen bonds. In the case of the anti-progesterone antibody (DB3), whose complex with five steroid ligands was determined by X-ray crystallography, a Trp residue occludes the binding site to form a “closed” conformation in an unbound state, whereas binding induces the orientation of the Trp to an “open” position. This is mediated by the rotation of Trp towards the D ring of the sterol. Further orientation of sterols in the binding pocket is driven by the formation of a hydrogen bond with the keto group of the sterol rings. This hydrogen-bond-dependent orientation, as well as charge complementarity, mediates the specificity in binding of structurally-related antigens [Jeffrey, P. D. et al. 40-50. J Mol Biol 248, 344-360 (1995)]. Similarly, antibody binding of estriol 3-(β-D-glucuronide) (EI3G) was demonstrated to be mediated via both hydrogen bonds and van der Waals interactions within a pocket that extends from the binding site to the interface of the light and heavy chains variable domains [Trinh et al. Structure 5, 937-948 (1997)]. Mechanistically, the interactions between aromatic residues and sterol ligands were shown in some antisterol antibodies to induce the opening of the binding interface, which is otherwise occluded by the aromatic residues in an unbound state [J. H. Arevalo, et al, J. Mol. Biol. 1994, 241, 663]. The formation of hydrogen bonds with the polar groups of the sterol rings controls the orientation of sterols in the binding pocket. This hydrogen-bond-dependent orientation mediates the specificity in binding of such structurally-related antigens as estrogens where unmatched hydroxyl groups result in a dramatic drop of hundreds to thousands fold in affinity [P. D. Jeffrey, J et al. J. Mol. Biol. 1995, 248, 344]. Such mechanisms underlying the small molecule-antibody interaction support the specificity and selectivity of estriol-antibody binding.

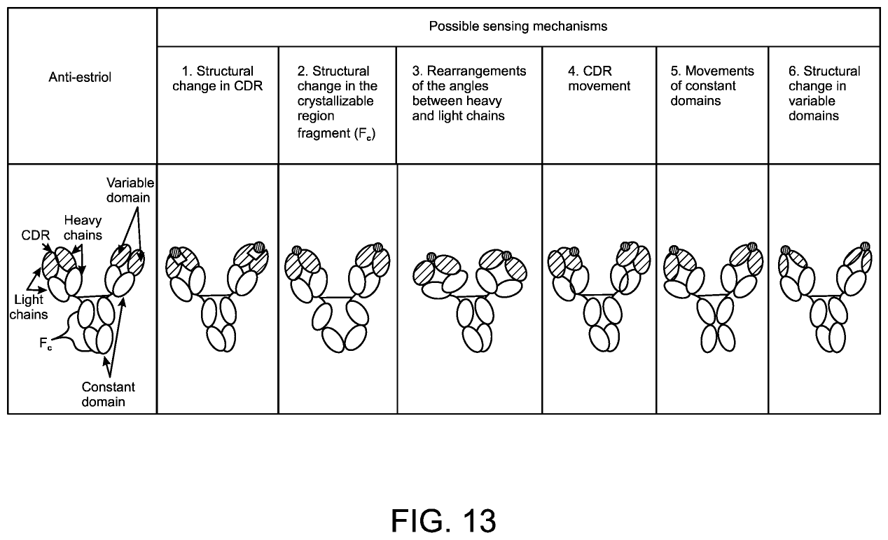

Next, the inventors consider the bioFET sensing signal in the case of small molecule-antibody interaction. In many cases, antigen binding induces a conformational change in the antibody to increase the binding efficiency by enhancing recognition or stabilization of the interaction. Suggested conformational changes are provided in FIG. 13 and can include: Structural changes in the antibody CDR (FIG. 13 item 1), structural changes in the crystallizable region fragment (Fc) (FIG. 13 item 2), rearrangements may include a change in the angles between the heavy chains and/or light chains (FIG. 13 item 3), CDR movements that affect the opening of pocket binding sites, side chain rearrangements (FIG. 13 item 4), or movement of the constant domains (FIG. 13 item 5). Yet, binding of small molecules such as steroids is expected to produce only mild structural and chemical changes and therefore pose challenges in identification and quantification of binding events by the antibody [M. M. Al Qaraghuli, et al. Front. Mol. Biosci. 2021, 8.]. For example, antigen binding to recombinant anti-testosterone Fab induced mild structural changes in the variable domains (FIG. 13 item 6) that resulted in tighter packing of the testosterone with the antibody [J. Valjakka, A. et al. J. Biol. Chem. 2002, 277, 44021]. Anti estradiol antibodies, on the other hand, showed no major conformational change upon ligand binding [C. Monnet, et al. J. Mol. Biol. 2002, 315, 699.]. An additional or alternative sensing mechanism can be related to the perturbation of the antibody charge distribution induced by the binding of the estriol [W. Shao et al. ACS Appl. Mater. Interfaces 2023, 15]. However, this is expected to be a limited effect due to the small size of the estriol. The available information regarding the interaction of sterols with antibodies can be used to hypothesize of the potential molecular mechanisms by which the estriol specific MNC BioFET recognizes and transduces signals in response to estriol. Estriol binding may induce conformational changes in the antibody, either by formation of new contact sites, adjustment of positions, or stabilization of regions, thereby inducing signal transduction by the sensor. Further, the positions of the polar groups complexed with the antibody, and the rearrangement in water molecules at the binding site, may induce charge alterations that may be detected by the sensor. However, both these mechanisms are expected to yield limited electrostatic perturbation to conclude a measurable sensing signal. Still, despite the challenges, the MNC bioFET provides excellent label-free and specific estriol sensing performance.

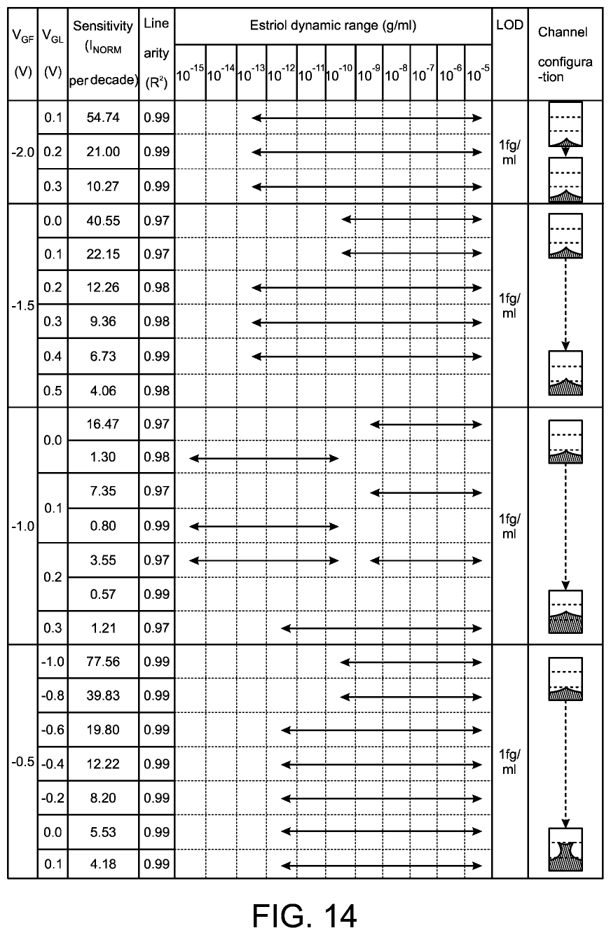

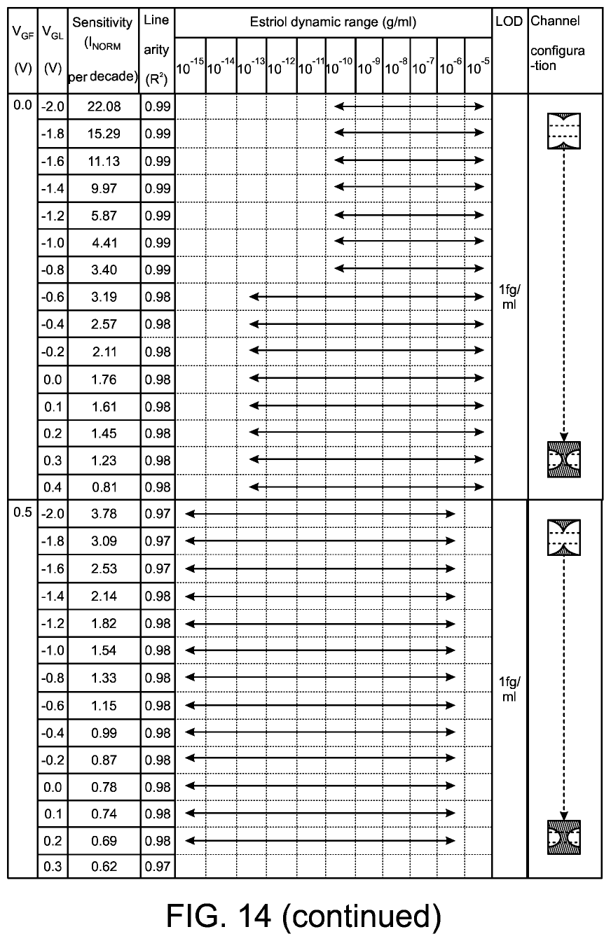

FIG. 14 presents Table 2 that summarizes the MNC bioFET real-time, specific, and label-free estriol sensing performance in terms of sensitivity, linearity, dynamic range, and limit-of-detection (LOD). The selected threshold values for linearity and dynamic range are 0.97 and at least 4 orders of magnitude, respectively (which also applies for the calibration curves presented in FIG. 12). The LOD for all considered channels is 1 fg/ml which is calculated in accordance with IUPAC definition [The IUPAC Compendium of Chemical Terminology. (International Union of Pure and Applied Chemistry (IUPAC), 2019). doi: 10.1351/goldbook]. Note the tuneability of the dynamic range and its dependency on channel configuration, with a highest dynamic range extending from 1 fg/ml to 10 μg/ml measured for V_{GF}=−1.5 V and V_{GL}=0.5 V. Generally, the onset of the dynamic range (i.e., the lowest estriol concentration of the dynamic range) depends mainly on V_{GF}. The dynamic range dependency on V_{GL} is via the sensitivity (=the slope): as the sensitivity decreases the onset of the dynamic range reaches lower concentration values as evident for V_{GF}=−1.5 V, for example. Also, interestingly, note the presence of piece-wise linearity as presented for V_{GF}=−1 V. Piece-wise linearity is not considered in conventional labelled sensing methods, such as ELISA, where a sensing signal is generated regardless of the molecular arrangement at the sensing area. However, with label-free FET sensors these molecular arrangements are important, as different arrangements can induce different electric fields which are not necessarily linear with analyte concentration. The highest sensitivity of 77.56 I_{Normalized}/dec is measured for V_{GF}=−0.5 V and V_{GL}=−1 V.

### Example 9

MNC biosensor biofunctionalized with anti-AFP antibodies (background measurements)

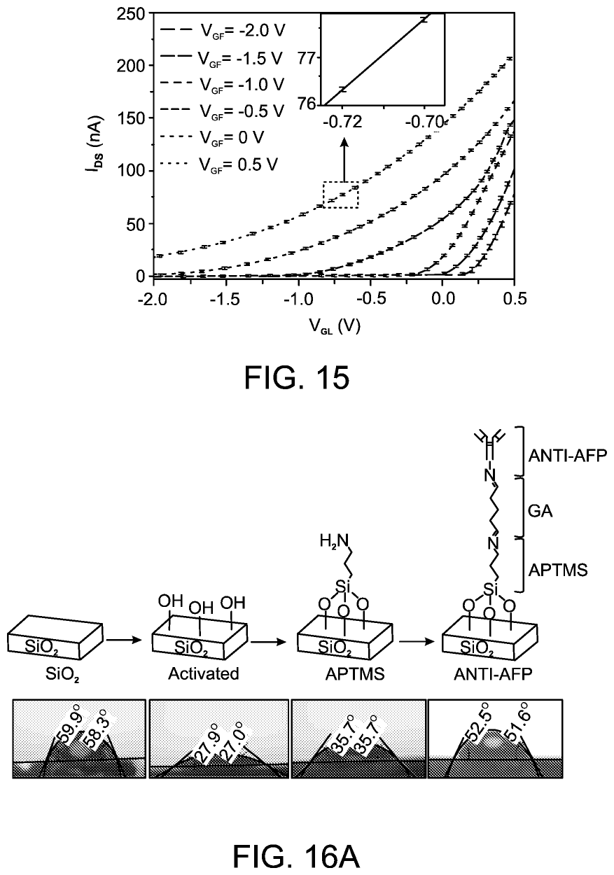

FIG. 15 shows the dependency of I_{DS} on V_{GL} for different V_{GF} values measured with a 0.5 μL drop of 1:100 diluted serum for an unmodified MNC device. Each curve was an average of 16 drops and each drop was measured 4 times with a total of 64 measurements. The inset shows a magnification of the data points showing the errors bar reflecting the standard deviations of the 64 measurements. The data presents an excellent repeatability of an unmodified MNC device in 1:100 diluted serum. The repeatability removes the concern of drop-to-drop pH fluctuations [Hal et al. Sensors and Actuators B 24-25, 201-205 (1995)], and establishes the potential stability of the quasi-reference electrode under possible drop-to-drop variations [Allen J. Bard, Electrochemical Methods: Fundamentals and Applications, 2nd Edition, John Wiley and Sons Inc., 2008]. The dependency of I_{DS} on V_{GL} and V_{GF} is detailed below in FIG. 17 for MNC biofunctionalized with AFP-antibodies.

### Example 10

Process of Si/SiO₂ Surface Biofunctionalization with Anti-AFP Antibodies

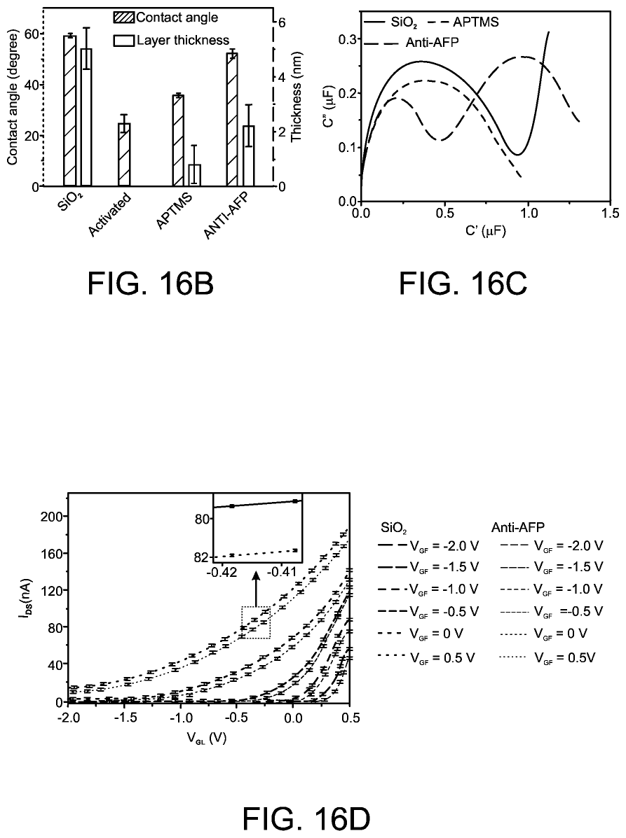

FIG. 16A illustrates the main steps towards sensing area biofunctionalization with anti-AFP. In short, the Si/SiO₂ sensing area was activated with piranha followed by surface chemical modification with APTMS linker molecules, and final surface-tethering of the anti-AFP antibodies (see Experimental procedure). FIG. 16A also presents the corresponding contact angle measurements. The surface modifications were developed and characterized using 1 cm×1 cm samples of silicon substrates decorated with 5 nm SiO₂ (FIG. 16B-16C). FIG. 16B presents contact angle measurements for Si/SiO₂ samples post piranha activation, post APTMS modification and surface biofunctionalization with anti-AFP antibodies. The measurements are performed at three locations on the samples with two measurements performed at each location, such that the data points and the error bars reflect the averages and standard deviations, respectively, of six measurements. The contact angle dependency on the various surface modifications follows behavior well documented previously [R. G. Frieser, J. Electrochem. Soc. 1974, 121, 669; Z.-Z. Liu, et al. Thin Solid Films 2008, 517, 635]. FIG. 16B also shows ellipsometry measurements performed on the same Si/SiO₂ samples. First, the SiO₂ sample is measured and the 5 nm SiO₂ thickness is validated. Afterwards, the sample is measured post APTMS modification where the SiO₂ is fixed and the APTMS thickness is fitted. Finally, the sample is measured post surface biofunctionalization with anti-AFP antibodies where both the SiO₂ and the APTMS thicknesses are fixed to the measured values and the antibody layer is fitted. The mean square error (MSE) for all measurements is smaller than 2. The thicknesses presented in FIG. 16C reflect the expected values of APTMS and the antibody layer [I. M. Bhattacharyya, et al. Nanoscale 2022, 14, 2837; K. Bierbaum, et al. Langmuir 1995, 11, 512; I. M. Bhattacharyya, et al. Adv. Electron. Mater. 2022, 2200399]. FIG. 16C presents the EIS measurements of the imaginary capacitance (C″) vs. the real capacitance (C′) for unmodified Si/SiO₂, post APTMS modification and post biofunctionalization with anti-AFP antibodies. Three distinct curves are shown, which reflect the effect of the modifications on the sensing area. The capacitance post APTMS modification is not significantly different from the unmodified sample, but the presence of an antibody layer is well reflected in the formation of an additional semi-circle characteristic of the generation of an additional layer. Finally, the biofunctionalization is applied to an MNC biosensor and FIG. 16D shows I_{DS} vs. V_{GL} for different V_{GF} values performed for unmodified, and MNC modified with anti-AFP antibodies. All the measurements are performed for 0.5 μL drops of 0.1 mM 7.4 pH PBS solution. Three drops are measured for the unmodified MNC biosensor, and three drops are measured for the modified MNC biosensor, and each drop is measured four times. FIG. 16D presents an excellent robustness and repeatability of a modified MNC device in 0.1 mM 7.4 pH PBS solution, as well as asserts the stability of the quasi-reference electrode under possible drop-to-drop variations [L. R. F. Allen J. Bard, Electrochemical Methods: Fundamentals and Applications, 2nd Edition, John Wiley And Sons Inc., 2008]. Finally, the repeatability addresses the concern of possible drop-to-drop pH fluctuations [R. E. G. Van Hal, J. C. T. Eijkel, P. Bergveld, Sensors Actuators B 1995, 24-25, 201].

### Example 11

I_{DS}-V_{GL} for Selected V_{GF} Values for MNC Biosensor Modified with Anti-AFP Molecules

FIG. 17A presents I_{DS}-V_{GL} for the selected 6 V_{GF} values of MNC biosensor modified with anti-AFP measured in 1:100 diluted serum. The contact angle of anti-AFP modified chip is 52°±0.5° and 2.2±0.8 nm is the measured thickness of the anti-AFP layer.

The data points and the error bars (see inset) reflect the averages and standard deviations, respectively, of 14 measured drops where each drop is measured 3 times. Note the excellent repeatability and robustness of the biofunctionalized MNC biosensor in 1:100 diluted serum. Furthermore, the repeatability reflects the buffering capability of the 0.5 μL drops of 1:100 diluted serum [R. E. G. Van Hal, J. C. T. Eijkel, P. Bergveld, Sensors Actuators B 1995, 24-25, 201], as well as the stability of the quasi-reference electrode under possible drop-to-drop variations [L. R. F. Allen J. Bard, Electrochemical Methods: Fundamentals and Applications, 2nd Edition, John Wiley And Sons Inc., 2008]. FIG. 17B presents the corresponding second derivatives of the curves presented in FIG. 17A where each peak represents the excitation of a conducting channel [I. M. Bhattacharyya, et al. Nanoscale 2022, 14, 2837; A. Ortiz-Conde, et al. Microelectron. Reliab. 2002, 42, 583]. For V_{GF}=0 V and 0.5 V, top and middle channels were normally-on, and the back channel was excited for higher V_{GL} values. Note that lower V_{GF} values force a higher voltage for the excitation of the back channel. I_{DS} is zero for V_{GF}=−0.5 V and low V_{GL}, the middle channel is excited upon V_{GL} increase (at V_{GL}~−0.6 V), and the top channel is excited afterwards (at V_{GL}~0.4 V). Obviously, lower V_{GF} values shift the excitation of the middle and top channels to higher V_{GL} values. Also, FIG. 17B illustrates the excitation sequence of the 3 channels for the various applied voltages. Importantly, as described above, although 3 channels are discussed, in practice each combination of V_{GL} and V_{GF} induces the formation of I_{DS} of a different shape and size.

The measurements were performed in 1:100 diluted serum, which implies protein background concentrations of 600-800 μg/ml (primarily albumin and globulins). More control measurements are provided in FIG. 17C. FIG. 17C(i)-17C(iv) show the non-specific response of the MNC biosensor for the introduction of AFP to an unmodified MNC biosensor, the introduction of AFP to an APTMS-modified MNC biosensor, and for the introduction of PSA and hCG to anti-AFP-modified MNC biosensors. The measurements are performed for 0.5 μL drops of 1:100 diluted serum spiked with the respective target molecule concentration. In each case, the target molecule concentrations for hCG are 0.27 nM, 2.70 nM and 27.0 nM and for PSA those are 0.35 nM, 3.50 nM and 35.0 nM in accordance with the 3 highest concentrations selected for AFP-specific sensing (0.11 nM, 1.05 nM and 10.5 nM). The I_{DS}-V_{GL} curves are very dense and the distinction between the different concentrations can be ascertained from the insets, where every data point is the average of 4 measurements performed for each drop (=concentration) and the error bars represent the corresponding standard deviations. In all cases, the negligible non-specific signals and the excellent repeatability remove the concerns of: 1) a sensing signal due to serum pH fluctuations induced by the presence of biomolecules [R. E. G. Van Hal, J. C. T. Eijkel, P. Bergveld, Sensors Actuators B 1995, 24-25, 201], 2) a sensing signal originating from physical adsorption of target molecules on the quasi-reference electrode, 3) a sensing signal due to effect of drop-to-drop variations on the quasi-reference electrode potential [L. R. F. Allen J. Bard, Electrochemical Methods: Fundamentals and Applications, 2nd Edition, John Wiley And Sons Inc., 2008], and 4) a sensing signal due to non-specific adsorption of target molecules on the MNC biosensor sensing area.

### Example 12

I_{DS}-V_{GL} for Selected V_{GF} Values for Different Concentrations of AFP Molecules

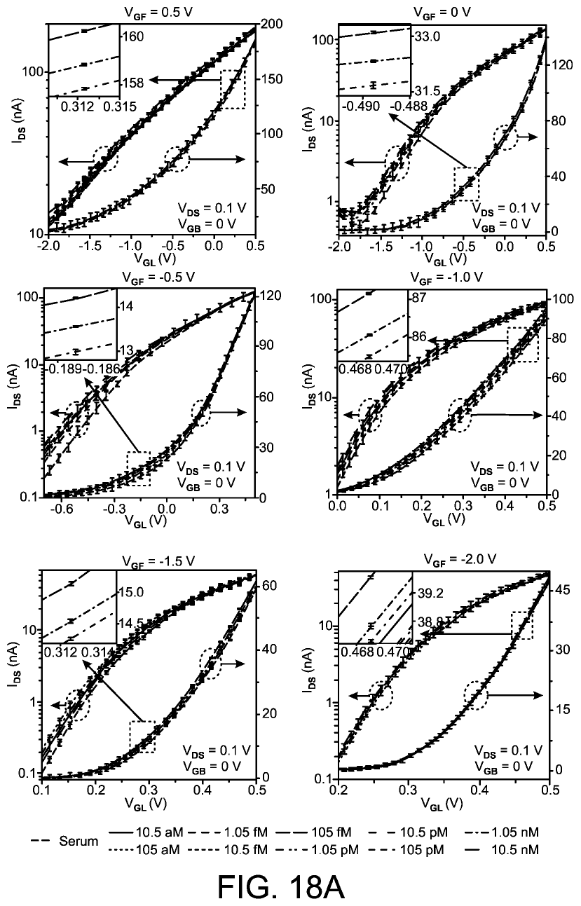

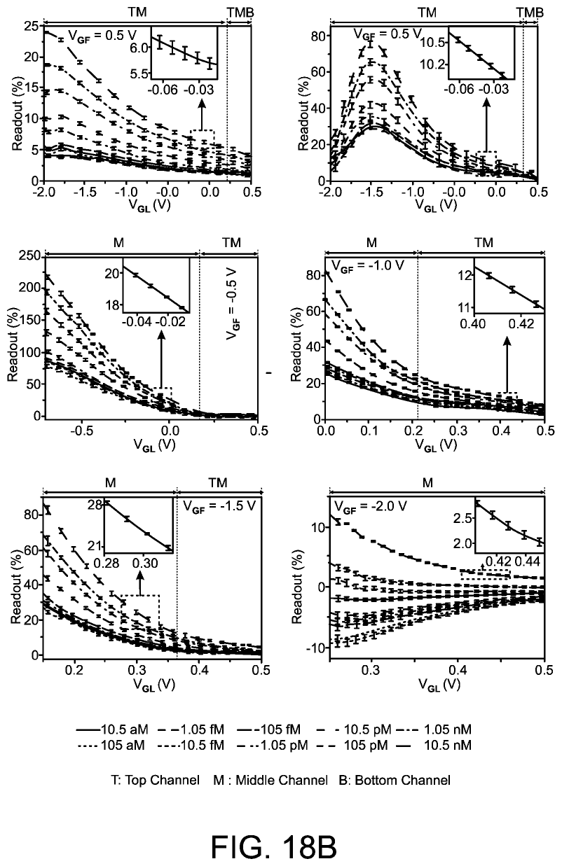

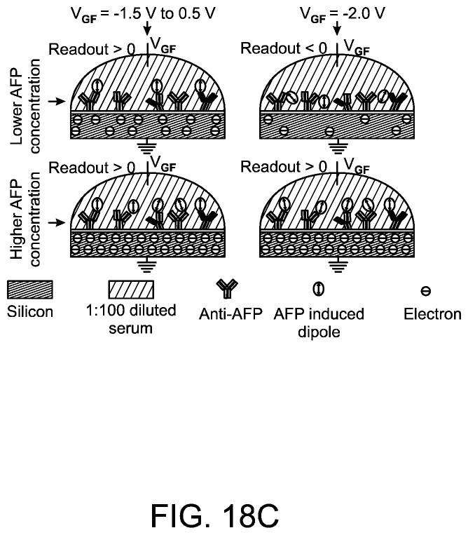

Next, real-time, specific, and label-free sensing of AFP molecules, from 0.5 μL drops of 1:100 diluted serum spiked with various AFP concentrations, was demonstrated with the MNC biosensor. FIG. 18A shows I_{DS}-V_{GL} curves for the 6 selected V_{GF} values, on both linear and logarithmic scales, and for all 10 AFP concentrations. The data points and the error bars (see insets) reflect the averages and standard deviations, respectively, of 4 measurements performed for each drop (=concentration). In order to confirm that the measured I_{DS}-V_{GL} curves do reflect specific signals, the following procedure was followed. First, it is ensured that the variation between one drop to the second (variation between successive AFP concentrations) is not due to the natural variation between two successive drops. Hence, the I_{DS} difference between two adjacent concentrations, for each V_{GF} and V_{GL} values, is required to be higher than the average difference between the 14 drops of FIG. 17A. Second, it is ensured that the variation between one drop to the next is not due to non-specific signals. Hence, the I_{DS} difference between a given AFP concentration and the baseline (diluted serum without AFP), for each V_{GF} and V_{GL} values, is required to be higher than the corresponding non-specific measurements presented in FIG. 17C. In this manner the following concerns are removed: sensing signals originating from pH fluctuations induced by the target molecules or drop-to-drop variation in pH [R. E. G. Van Hal, J. C. T. Eijkel, P. Bergveld, Sensors Actuators B 1995, 24-25, 201], non-specific adsorption on the quasi-reference electrode and the MNC biosensor sensing area, and the effect of drop-to-drop variation on the quasi-reference electrode potential [L. R. F. Allen J. Bard, Electrochemical Methods: Fundamentals and Applications, 2nd Edition, John Wiley And Sons Inc., 2008.

FIG. 17B shows the Readout curves corresponding to the I_{DS}-V_{GL} curves of FIG. 17A. The Readout was calculated as (I_{DS}^{AFP}−I_{DS}^{baseline})/I_{DS}^{baseline}, where I_{DS}^{AFP} is the measured I_{DS} for a given AFP concentration and for set values of V_{GF} and V_{GL}, and I_{DS}^{baseline} is the I_{DS} measured for 1:100 diluted serum without AFP spiking. Importantly, the x-axis scales were adjusted to ensure that values reflecting a null I_{DS} (noise level) are not considered. The relevant excited channels are also marked in FIG. 17B (T for top channel, M for middle channel, and B for back channel). First, the dependency of the 10.5 aM concentration, for V_{GF}=−0.5 V and V_{GL}=−0.7 V, is 75% which corresponds with an excited middle channel. Note how the Readout drops with increasing V_{GL} which corresponds to the widening of the middle channel. Also, the Readout for the same 10.5 aM concentration is just about nulled for V_{GF}=−2V and V_{GL}=0.2 V which corresponds also to an excited middle channel. However, the former middle channel is very different in shape from the latter. The latter middle channel is further removed from the front interface (V_{GF}=−2 V vs. V_{GF}=−0.5 V), and it is also considerably wider compared with the former channel (V_{GL}=0.2 V vs. V_{GL}=−0.7 V). This is the important merit of the MNC biosensor: the variety of available channels provides multitude of ways to couple the random electrostatic distribution of the biological interactions at the sensing area, and the consequential surface potential distribution, with the electrodynamics of the underlying conducing channels to provide a meaningful I_{DS}. Next, the effect of active sensing was demonstrated and discussed. All measured channels exhibit positive Readout values which imply an overall total positive charge at the sensing area, except the middle channel of V_{GF}=−2 V. For this middle channel of V_{GF}=−2 V the Readout switches polarity: for AFP concentration range of 10.5 fM-10.5 nM the Readout increases with AFP concentration increase, but it is negative for 10.5 fM-10.5 pM and positive for 105 pM-10.5 nM. The switch in Readout polarity suggests that the four gates do not only affect the transduction of the sensing area potential into I_{DS}, but also affects the interactions themselves, refereed to as active sensing. In other words, if the total charge distribution of the complexes at the sensing area is positive, then the Readout must be positive and cannot take negative values. As a corollary, a switch in Readout polarity suggests that the biological interactions at the sensing area are affected by the gating configuration (active sensing). The effect of V_{GL} on biological interactions does not seem probable as V_{GL} produces horizontal electric fields in the silicon device layer. On the other hand, V_{GF} affects the double layer (which is also affected by the biological entities and interactions) adjacent to the sensing area. Hence, V_{GF} determines the double layer electric field, the pH level and the ionic strength, all can potentially affect biological interactions at the sensing area [L. R. F. Allen J. Bard, Electrochemical Methods: Fundamentals and Applications, 2nd Edition, John Wiley And Sons Inc., 2008; I. M. Bhattacharyya, G. Shalev, ACS Sensors 2020, 5, 154]. Therefore, it is suggested that V_{GF}=−2 V produces a double layer environment which results in total negative charge and a Readout decrease. Note that V_{GF} also affects the Debye screening length at the sensing area (by the determination of surface ionic strength), but in this case the Readout is not expected to switch polarity. FIG. 18C illustrates one possible mechanism to induce a switch in Readout polarity which relates to the direction of the double layer electric field determined by V_{GF}. Such a switch in the polarity of the electric field can affect the orientation of the biological complexes and to generate either positive or negative Readout. Of course, the effect of the double layer electric field on the orientation of the target molecules also depends on the density of the surface-bound complexes, as presented in FIG. 18C.

Active sensing with the MNC biosensor is provided by the solution potential which affects the biological interactions indirectly by determining the conditions at the double layer. Still, more research is needed to underpin the mechanism by which V_{GF} affects the biological interactions at the sensing area.

### Example 13

### Readout Calibration Curves for AFP Molecules

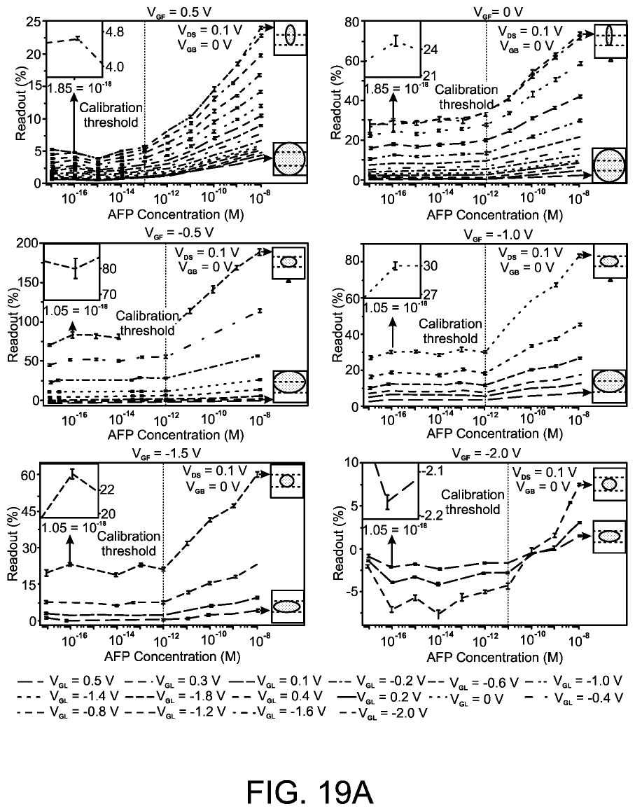

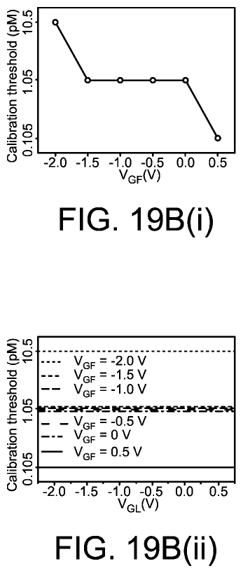

FIG. 19A shows the Readout calibration curves for the considered channel configurations. The illustrations on the right of the calibration curves show the cross-sections, midway between the source and drain, of the various channels visualizing the size, shape, and location of the corresponding channels. Note, that each line pattern represents a calibration curve obtained for a different channel configuration. Also, dashed vertical grey lines indicate the ‘calibration threshold’ of the calibration curves which marks the onset of the linear region of the dynamic range. Two interesting observations are in place. First, V_{GL} determines the sensitivity (the slope of the linear region) and lower V_{GL} generates higher sensitivity. This directly reflects the effect of channel configuration on sensing performance. Second, active sensing is allowed as V_{GF} affects the calibration threshold value. The dependency of the calibration threshold on V_{GF} is presented in FIG. 19B(i). Evidently, V_{GF} affects the calibration threshold significantly in the range of three orders of magnitude in AFP concentration from 105 fM to 10.5 pM. The importance of the calibration threshold is with respect to its association with K_{d}. K_{d} is defined in accordance with the law of mass action in equilibrium: [ligand]·[receptor]·kₒₙ=[ligand:receptor]·k_{off}, where [ligand] and [receptor] are the concentrations or activities of the reactants, [ligand:receptor] is the concentration or activity of the complexes, and kₒₙ and k_{off} are rate constants for the forward and reverse reactions, respectively. K_{d} is defined as K_{d}=([ligand]·[receptor])/[ligand:receptor], such that K_{d}=[ligand] reflect a state in which half of the receptors are unbound and half are bound to ligands [D. B. Finlay, S. B. Duffull, M. Glass, Br J Pharmacol. 2020, 177, 1472]. Therefore, experimentally, K_{d} is extracted from calibration curves by intersecting the ligand concentration with the middle of the calibration curve linear region, halfway between calibration threshold and saturation. However, need to keep in mind that this description does not apply accurately to the current work as the surface-bound anti-AFP antibodies, at the MNC biosensor sensing area, do not interact with all the AFP molecules present in the 0.5 μL drop, as the surface occupied by the drop is significantly greater than the sensing area. Still, following the conventional definition of K_{d}, as provided above, a shift in the calibration thresholds presented in FIG. 19A directly implies a shift in K_{d}, and the calibration threshold shift from 105 fM to 10.5 pM suggests a shift from a higher binding affinity to a lower binding affinity, respectively. FIG. 19B(ii) shows the lack of dependency of the calibration threshold value on V_{GL}. This lack of dependency is expected as V_{GL} affects solely the electron charge carriers in the silicon, and its effect on the potential of the solid-biological interface is negligible. On the other hand, the dependency of the calibration threshold on V_{GF} is expected, as V_{GF} determines Ñ±0 and the double layer conditions in terms of pH, ionic strength and electric field, each of these can potentially affect the ligand-receptor interaction.

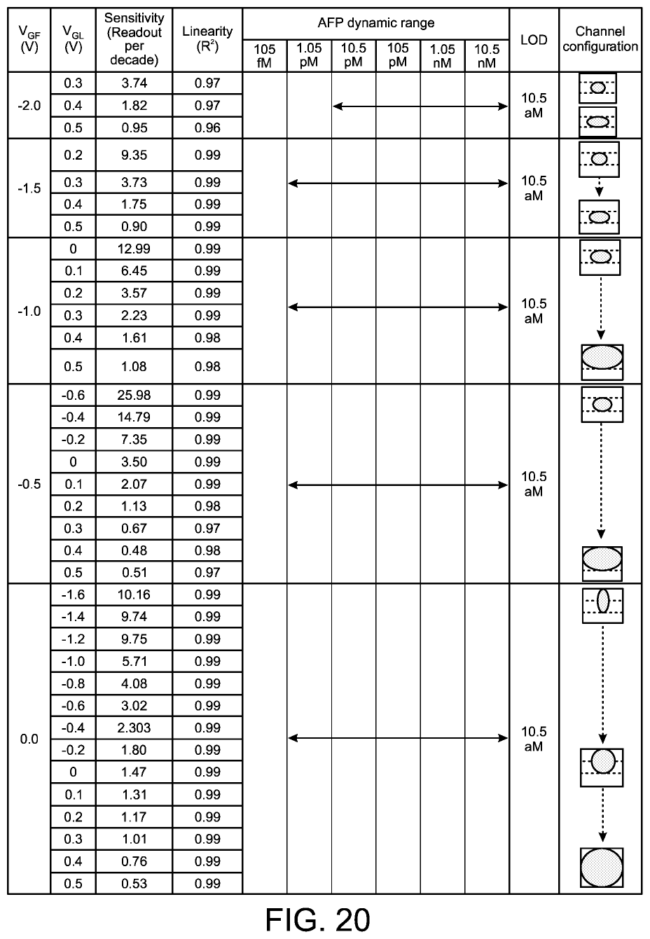

A summary of the sensing performance is provided in FIG. 20 that discloses Table 3, which presents the dependency of the sensitivity, linearity (R²), dynamic range, and LOD on channel configuration. The sensitivity, which is the slope of the linear fit in units of Readout per decade of concentration (% dec⁻¹), and the LOD, defined as the lowest concentration with an I_{DS} greater than the average I_{DS} plus three standard deviations of the background diluted serum, are extracted in accordance with the IUPAC conventions [M. Nič, J. Jirát, et al. IUPAC Compendium of Chemical Terminology: Gold Book, IUPAC, Research Triagle Park, NC, 2.1.0., 2009]. The requirements for the presented performances are a dynamic range of at least four orders of magnitude, and R² higher or equal to 0.96. Highest sensitivity of 25.98 Readout/dec, for a dynamic range of 1.05 pM-10.5 nM, was measured for a narrow middle channel (V_{GF}=−0.5 V, V_{GL}=−0.6 V). Note that the sensitivity exhibits a maxima behavior in terms of V_{GF} (the sensitivity decreases for V_{GF} smaller or greater than −0.5 V), and it is always higher for a narrow middle channel (small V_{GL} values). Therefore, V_{GF}=−0.5 V provides the optimal double layer in terms of sensitivity, and V_{GL}=−0.6 V provides the most efficient transduction of the surface potential, induced by the biological interactions, to an electronic signal. On the other hand, V_{GF}=0.5 V provides the highest dynamic range from 105 fM to 10.5 nM, and, similar to the other V_{GF} values, lower V_{GL} values conclude an enhanced sensitivity.

The dependency of the sensing performance on channel configuration is shown. The criteria for channel configuration selection are R²≥0.96 and a dynamic range of at least 4 orders of magnitude. The LOD and the dynamic range are extracted in accordance with the IUPAC definition [M. Nič, et al. IUPAC Compendium of Chemical Terminology: Gold Book, IUPAC, Research Triagle Park, NC, 2.1.0., 2009]

It should be noted that 105 fM, 1.05 pM, 10.5 pM, 105 pM, 1.05 nM, and 10.5 nM AFP corresponds to 10 pg/ml, 100 pg/ml, 1 ng/ml, 10 ng/ml, 100 ng/ml and 1 ug/ml AFP, respectively.

1.-62. (canceled)

63. A bio-transistor system comprising:
(a) at least one transistor unit comprising:
(i) at least one channel;
(ii) source and drain electrodes;
(iii) at least one gate electrode; and
(iv) at least one active region located in proximity to the channel region and carrying at least one affinity moiety, wherein each affinity moiety is specific for a target molecule; said at least one active region is configured for accepting at least one sample; and
(v) at least one additional electrode positioned to be in electrical contact with the sample; and
(b) a control system comprising at least one processor and memory circuitry, wherein said control system is configured and operable for performing one or more measurements of the sample, wherein each measurement comprises maintaining a selected electric potential on the at least one additional electrode, and determining current transmission profile through the at least one channel with respect to potential variation of said at least one gate electrode;
said control unit thereby configured to determine data on the presence and/or quantity of said at least one target molecule(s) in the sample.

64. The bio-transistor system of claim 63, wherein at least one of:
(a) said control system is configured to performed two or more measurements utilizing two or more different selected electric potentials applied to the at least one additional electrode;
(b) said control system comprises pre-stored calibration data comprising data on electric transmission through the channel for given gate electrode potential with respect to one or more selected electric potentials applied to the at least one additional electrode;
(c) said control system comprises pre-stored calibration data comprising data on electric transmission trough the channel with respect to variation of the gate electrode potential;
(d) comprising a plurality of two or more transistor units comprising respective plurality of two or more active regions carrying two or more different or identical types of affinity moieties;
(e) wherein the active region is separated from the channel region by an electrical insulator layer;
(f) wherein the at least one gate electrode is electrically insulated from the active region; and
(g) wherein the control system comprises at least one electrical circuit coupled vie electrical connection to the transistor unit and configured to provide selected electric potentials to electrodes of the transistor unit.

65. The bio-transistor system of claim 63, at least one of:
(a) wherein said at least one affinity moiety comprises at least one of: an amino acid-based molecule, a small molecule, a nucleic acid-based molecule, a carbohydrate-based molecule, a lipid-based molecule or any combination thereof, and wherein each of said affinity moiety specifically binds a target molecule in said sample;
(b) wherein said at least one affinity moiety comprises, or is derived from a component of an affinity pair, said affinity pair comprises at least one of: receptor-ligand, antibody-antigen, enzyme-substrate, aptamer-aptamer target, or any combination thereof;
(c) wherein said target molecule comprises at least one of: a small molecule, an amino acid-based molecule, a nucleic acid-based molecule, a carbohydrate-based molecule, a lipid-based molecule or any combination thereof;
(d) wherein said target molecule comprises, or is derived from a component of an affinity pair, said affinity pair comprises at least one of: receptor-ligand, antibody-antigen, enzyme-substrate, aptamer-aptamer target, or any combination thereof; and
(e) wherein said at least one affinity pair is a receptor-ligand affinity pair.

66. The bio-transistor system of claim 63, wherein said target is at least one ligand comprising a small molecule chemical substance, and wherein said affinity moiety comprises an amino-acid based receptor molecule for said ligand; optional, at least one of:
(a) wherein said chemical substance is toxic for at least one eukaryotic organism;
(b) wherein said chemical substance is at least one organophosphate;
(c) wherein said organophosphate comprises at least one of pesticides, herbicides, and/or chemical warfare agent(s); and
(d) wherein said at least one organophosphate is diethylcyanophosphonate (DCNP).

67. The bio-transistor system of claim 66, wherein at least one of:
(a) wherein said receptor is hexafluoroisopropanol (HFIP), and wherein said affinity moiety comprises HFIP or any functional fragment thereof;
(b) wherein said at least one sample is a biological sample and/or environmental sample; and
(c) wherein said sample is an environmental sample.

68. The bio-transistor system of claim 63, wherein at least one of:
(a) said at least one affinity pair is an antigen-antibody affinity pair; and
(b) wherein said target is at least one antigen comprising a small molecule steroid compound, and wherein said affinity moiety comprises an antibody specific for said antigen.

69. The bio-transistor system of claim 63, wherein at least one of:
(I) said small molecule steroid compound is naturally produced by at least one eukaryotic organism; optionally, at least one of:
(a) wherein said small molecule steroid compound is of the estrogen family;
(b) wherein said small molecule steroid compound is Estriol;
(II) wherein said antibody is an anti-Estriol antibody or any functional fragments thereof; and
(III) wherein said at least one sample is a biological sample and/or environmental sample, optionally, wherein the sample is a biological sample.

70. The bio-transistor system of claim 63, wherein said at least one sample further comprises a candidate compound that modulates the interaction between said at least one affinity moiety and said target molecule, wherein said candidate compound comprises at least one of: an amino acid-based, a nucleic acid-based, a small molecule-based, a carbohydrate-based, a lipid-based affinity molecule or any combination thereof.

71. A battery comprising two or more of the bio-transistor system as defined in claim 63, wherein at least one of said two or more of the bio-transistor systems comprise at least one affinity moiety for at least one maternal serum marker, said battery comprising:
(a) at least one bio-transistor system comprising at least one affinity moiety specific for Estriol, and
(b) at least one of:
(i) at least one bio-transistor system comprising at least one affinity moiety specific for alpha-fetoprotein (AFP); and
(ii) at least one bio-transistor system comprising at least one affinity moiety specific for human chorionic gonadotropin (hCG).

72. A method for determining presence and/or quantity of at least one target molecule in at least one sample, comprising:
(a) contacting the at least one sample with a bio-transistor having an active region carrying at least one affinity moiety, or a battery comprising at least two of said bio-transistors, wherein each affinity moiety is specific for a target molecule;
(b) performing one or more measurements, each measurement comprising: applying a selected electric potential on the sample, and determining current transmission profile through a channel of the bio-transistor with respect to potential variation of at least one gate electrode of the bio-transistor; and
(c) processing data on the current transmission through the channel for one or more selected gate potential and one or more selected electric potential values applied on the sample and determining presence and/or quantity of said at least one target molecule(s) in accordance with pre-stored calibration data.

73. The method of claim 72, wherein at least one of:
(I) wherein the bio-transistor comprising:
(a) at least one channel;
(b) source and drain electrodes;
(c) at least one gate electrode; and
(d) at least one additional electrode positioned to be in electrical contact with the sample;
wherein the at least one active region is located in proximity to the channel region, separated from the channel region by an electrically insulating layer, and wherein the control unit thereby configured to determine data on presence and/or quantity of at least one target molecule/s in the sample; and
(II) wherein said performing one or more measurements comprises performing two or more measurements, wherein each measurement comprises applying respective selected different potential on the sample.

74. The method of claim 72, wherein at least one of:
(a) wherein said target is at least one ligand comprising a small molecule chemical substance, and wherein said affinity moiety comprises an amino-acid based receptor molecule for said ligand; optionally, at least one of:
(i) wherein said chemical substance is toxic for at least one eukaryotic organism;
(ii) wherein said chemical substance is at least one organophosphate;
(iii) wherein said organophosphate comprise at least one of pesticides, herbicides, and/or chemical warfare agent/s; and
(iv) wherein said at least one organophosphate is diethylcyanophosphonate (DCNP);
(b) wherein said receptor is hexafluoroisopropanol (HFIP);
(c) wherein said at least one sample is a biological sample and/or environmental sample; and
(d) for detecting organophosphate contamination in a sample.

75. The method of claim 73, wherein at least one of:
(a) said target is at least one antigen comprising a small molecule steroid compound, and wherein said affinity moiety comprises an antibody specific for said antigen;
(b) wherein said small molecule steroid compound is naturally produced by at least one eukaryotic organism; optionally, wherein said small molecule steroid compound is of the estrogen family;
(c) wherein said small molecule steroid compound is Estriol, and wherein said antibody is an anti-Estriol antibody or any functional fragments thereof; and
(d) wherein said sample is a biological sample.

76. A diagnostic method for determining a physiological and/or environmental condition or state of a media and/or a habitat and/or a subject, comprising:
(a) contacting the at least one sample with a bio-transistor having an active region carrying at least one affinity moiety, or a battery comprising at least two of said bio-transistors, wherein each affinity moiety is specific for a target molecule;
(b) performing one or more measurements, each measurement comprising: applying a selected electric potential on the sample, and determining current transmission profile through a channel of the bio-transistor with respect to potential variation of at least one gate electrode of the bio-transistor;
(c) processing data on the current transmission through the channel for one or more selected gate potential and one or more selected electric potential values applied on the sample; and
(d) determining presence and/or quantity of the target molecule(s) in accordance with pre-stored calibration data, thereby obtaining a target molecule value for said sample; and
(e) determining that the subject and/or media and/or habitat display said physiological and/or environmental condition or state, if the at least one target molecule value obtained for said sample in step (d), is positive or negative with respect to a reference target molecule value pre-determined for said physiological and/or environmental condition or state, or with respect to a target molecule value determined for at least one control sample.

77. The diagnostic method of claim 76, wherein at least one of:
(I) said bio-transistor system comprising:
(a) at least one transistor unit comprising:
(i) at least one channel;
(ii) source and drain electrodes;
(iii) at least one gate electrode; and
(iv) at least one active region located in proximity to the channel region and carrying at least one affinity moiety, wherein each affinity moiety is specific for a target molecule;
said at least one active region is configured for accepting at least one sample; and
(v) at least one additional electrode positioned to be in electrical contact with the sample; and
(b) and a control system comprising at least one processor and memory circuitry, wherein said control system is configured and operable for performing one or more measurements of the sample, wherein each measurement comprises maintaining a selected electric potential on the at least one additional electrode, and determining current transmission profile through the at least one channel with respect to potential variation of said at least one gate electrode;
said control unit thereby configured to determine data on the presence and/or quantity of said at least one target molecule(s) in the sample; and
(II) wherein said environmental condition or state of a media and/or a habitat affects at least one pathological condition and/or at least one health condition in said subject.

78. The method of claim 76, wherein said bio-transistor system and/or battery comprise at least one affinity moiety comprising HFIP, said affinity moiety is specific for DCNP, and wherein said method is for the diagnosis of organophosphate(s) contamination in a media and/or habitat optionally, wherein said method is for the diagnosis of a pathological condition in a subject caused by exposure to a media and/or habitat contaminated by said organophosphate(s).

79. The method of claim 76, wherein said bio-transistor system and/or battery comprise at least one affinity moiety specific for estriol, said affinity moiety comprising anti-estriol antibody, and wherein step (d) results in obtaining the estriol value for a maternal serum sample.

80. The method of claim 76, wherein at least one of said two or more of the bio-transistor systems of said battery comprise at least one affinity moiety for at least one maternal serum marker, said battery comprising:
(i) at least one bio-transistor system comprising at least one affinity moiety specific for Estriol; and at least one of:
(ii) at least one bio-transistor system comprising at least one affinity moiety specific for alpha-fetoprotein (AFP); and
(iii) at least one bio-transistor system comprising at least one affinity moiety specific for human chorionic gonadotropin (hCG);
and wherein step (d) of said method results in obtaining the estriol value for at least one maternal serum sample, and optionally of at least one of, the AFP value and/or the hCG value of said at least one maternal serum sample, optionally, wherein said method is for the diagnosis of neural tube defects and/or genetic abnormalities in a fetus.

81. A screening method for identifying a compound that modulates the interaction of an affinity moiety with a target molecule in at least one sample, comprising:
(a) contacting said at least one sample with a bio-transistor system as defined in claim 63, in the presence and the absence of at least one candidate compound, said bio-transistor having an active region carrying at least one affinity moiety, wherein each affinity moiety is specific for a target molecule;
(b) performing one or more measurements for each sample, each measurement comprising: applying a selected electric potential on the sample, and determining current transmission profile through a channel of the bio-transistor with respect to potential variation of at least one gate electrode of the bio-transistor; and
(c) processing data on the current transmission through the channel for one or more selected gate potential and one or more selected electric potential values applied on the sample; and
(d) determining presence and/or quantity of the target molecule(s) in accordance with pre-stored calibration data, thereby determining a target molecule value for said sample in the presence of said candidate compound, and/or a target molecule value in the absence of said candidate compound;
(e) determining that said candidate compound is a modulator of the interaction between the affinity moiety and the target molecule, if the target molecule value obtained for said sample in the presence of said candidate compound is different from the target molecule value obtained in the absence of said candidate compound.

82. A diagnostic kit comprising:
(a) at least one bio-transistor system as defined in claim 63, and optionally, at least on of:
(b) at least one control sample; and
(c) at least one at least one therapeutic agent;
wherein said bio-transistor system comprises:
(i) at least one transistor unit comprising:
i. at least one channel;
ii. source and drain electrodes;
iii. at least one gate electrode;
iv. at least one active region located in proximity to the channel region and carrying at least one affinity moiety, wherein each affinity moiety is specific for a target molecule;
said at least one active region is configured for accepting at least one sample; and
V. at least one additional electrode positioned to be in electrical contact with the sample; and
(ii) a control system comprising at least one processor and memory circuitry, wherein said control system is configured and operable for performing one or more measurements of the sample, wherein each measurement comprises maintaining a selected electric potential on the at least one additional electrode, and determining current transmission profile through the at least one channel with respect to potential variation of said at least one gate electrode;
said control unit thereby configured to determine on the presence and/or quantity of said target molecule(s) in the sample; optionally, at least one of:
(I) wherein said method is for the diagnosis of organophosphate/s contamination in a media and/or habitat and/or for the diagnosis of a pathological condition in a subject caused by exposure to a media and/or habitat contaminated by said organophosphate/s; and
(II) wherein said method is for the diagnosis of neural tube defects and/or genetic abnormalities in a fetus.
